SEQUENCE LISTING (130 sequences)

SEQ ID NO: 1 - protein, 181 aa, Homo sapiens
EPPTQKPKKIVNAKKDVVNTKMFEELKSRLDTLAQEVALLKEQQALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 2 - protein, 181 aa, Mus musculus
ESPTPKAKKAANAKKDLVSSKMFEELKNRMDVLAQEVALLKEKQALQTVCLKGTKVNLKCLLAFTQPKTFHEASEDCISQGGTLGTPQSELENEALFEYARHSVGNDANIWLGLNDMAAEGAWVDMTGGLLAYKNWETEITTQPDGGKAENCAALSGAANGKWFDKRCRDQLPYICQFAIV

SEQ ID NO: 3 - protein, 181 aa, Gallus gallus
QQNGKGRQKPAASKKDGVSLKMIEDLKAMIDNISQEVALLKEKQALQTVCLKGTKIHLKCFLAFSESKTYHEASEHCISQGGTLGTPQGGEENDALYDYMRKSIGNEAEIWLGLNDMVAEGKWVDMTGSPIRYKNWETEITTQPDGGKLENCAALSGVAVGKWFDKRCKEQLPYVCQFMIV

SEQ ID NO: 4 - protein, 181 aa, Bos taurus
ETPTPKAKKAANAKKDAVSPKMLEELKTQLDSLAQEVALLKEQQALQTVCLKGTKVHMKCFLAFVQAKTFHEASEDCISRGGTLGTPQTGSENDALYEYLRQSVGSEAEVWLGFNDMASEGSWVDMTGGHIAYKNWETEITAQPDGGKVENCATLSGAANGKWFDKRCRDKLPYVCQFAIV

SEQ ID NO: 5 - protein, 178 aa, Salmo salar
QQTSSKKKGGKKDAENNAAIEELKKQIDNIVLELNLLKEQQALQSVCLKGIKIIGKCFLADTAKKIYHTAYDDCIAKGGTISTPLTGDENDQLVDYVRRSIGPEEHIWLGINDMVTEGEWLDQAGTNLRFKNWETDITNQPDGGRTHNCAILSTTANGKWFDESCRVEKASVCEFNIV

SEQ ID NO: 6 - protein, 178 aa, Xenopus tropicalis
QQNGKKNKQNNKDVVSMKMYEDLKKKVQNIEEDVIHLKEQQALQTICLKGMKIYNKCFLAFNELKTYHQASDVCFAQGGTLSTPETGDENDSLYDYVRKSIGSSAEIWIGINDMATEGTWLDLTGSPISFKHWETEITTQPDGGKQENCAALSASAIGRWFDKNCKTELPFVCQFSIV

SEQ ID NO: 7 - protein, 177 aa, Danio rerio
EQSLTKRKNGKKESNSAAIEELKKQIDQIIQDLNLLKEQQALQTVCLKGFKIPGKCFLVDTVKKDFHSANDDCIAKGGILSTPMSGHENDQLQEYVQQTVGPETHIWLGVNDMIKEGEWIDLTGSPIRFKNWESEITHQPDGGRTHNCAVLSSTANGKWFDEDCRGEKASVCQFNIV

SEQ ID NO: 8 - protein, 179 aa, Bos Taurus
QTSCHASKFKARKHSKRRVKEKDGDLKTQVEKLWREVNALKEMQALQTVCLRGTKFHKKCYLAAEGLKHFHEANEDCISKGGTLVVPRSADEINALRDYGKRSLPGVNDFWLGINDMVAEGKFVDINGLAISFLNWDQAQPNGGKRENCALFSQSAQGKWSDEACHSSKRYICEFTIPQ

SEQ ID NO: 9 - protein, 165 aa, Carcharhius springeri
KPSKSGKGKDDLRNEIDKLWREVNSLKEMQALQTVCLKGTKIHKKCYLASRGSKSYHAANEDCIAQGGTLSIPRSSDEGNSLRSYAKKSLVGARDFWIGVNDMTTEGKFVDVNGLPITYFNWDRSKPVGGTRENCVAASTSGQGKWSDDVCRSEKRYICEYLIPV

SEQ ID NO: 10 - protein, 52 aa, Homo sapiens
EPPTQKPKKIVNAKKDVVNTKMFEELKSRLDTLAQEVALLKEQQALQTVCLK

SEQ ID NO: 11 - protein, 52 aa, Mus musculus
ESPTPKAKKAANAKKDLVSSKMFEELKNRMDVLAQEVALLKEKQALQTVCLK

SEQ ID NO: 12 - protein, 52 aa, Gallus gallus
QQNGKGRQKPAASKKDGVSLKMIEDLKAMIDNISQEVALLKEKQALQTVCLK

SEQ ID NO: 13 - protein, 52 aa, Bos Taurus
ETPTPKAKKAANAKKDAVSPKMLEELKTQLDSLAQEVALLKEQQALQTVCLK

SEQ ID NO: 14 - protein, 49 aa, Salmo salar
QQTSSKKKGGKKDAENNAAIEELKKQIDNIVLELNLLKEQQALQSVCLK

SEQ ID NO: 15 - protein, 49 aa, Xenopus tropicalis
QQNGKKNKQNNKDVVSMKMYEDLKKKVQNIEEDVIHLKEQQALQTICLK

SEQ ID NO: 16 - protein, 48 aa, Danio rerio
EQSLTKRKNGKKESNSAAIEELKKQIDQIIQDLNLLKEQQALQTVCLK

SEQ ID NO: 17 - protein, 52 aa, Bos Taurus
QTSCHASKFKARKHSKRRVKEKDGDLKTQVEKLWREVNALKEMQALQTVCLR

SEQ ID NO: 18 - protein, 38 aa, Carcharhinus springeri
KPSKSGKGKDDLRNEIDKLWREVNSLKEMQALQTVCLK

SEQ ID NO: 19 - protein, 35 aa, Artificial Sequence
  Synthetic
  MISC_FEATURE (1)..(8): Xaa can be any naturally occurring amino acid
  MISC_FEATURE (10)..(13): Xaa can be any naturally occurring amino acid
  MISC_FEATURE (14)..(14): Xaa is a hydrophobic residue
  misc_feature (15)..(15): Xaa can be any naturally occurring amino acid
  MISC_FEATURE (17)..(18): Xaa can be any naturally occurring amino acid
  MISC_FEATURE (21)..(22): Xaa can be any naturally occurring amino acid
  MISC_FEATURE (26)..(26): Xaa can be any naturally occurring amino acid
  MISC_FEATURE (35)..(35): Xaa can be any naturally occurring amino acid
XXXXXXXXLXXXXXXLXXEVXXLKEXQALQTVCLX

SEQ ID NO: 20 - protein, 15 aa, Artificial Sequence
  Synthetic
EELICSRLDTLAQEV

SEQ ID NO: 21 - protein, 152 aa, Artificial Sequence
  Synthetic
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 22 - protein, 204 aa, Artificial Sequence
  Synthetic
EGPTQKPKKIVNAKKDVVNTKMFEELKSRLDTLAQEVALLKEQQALQTVSLKRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 23 - protein, 200 aa, Artificial Sequence [sequence omitted]

SEQ ID NO: 24 - protein, 200 aa, Artificial Sequence
  Synthetic
EGPTQKPKKIVNAKKDVVNTKMFEELKSRLDTLAQEVALLKEQQALQTRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 25 - protein, 199 aa, Artificial Sequence
  Synthetic
EGPTQKPKKIVNAKKDVVNTKMFEELKSRLDTLAQEVALLKEQQALQRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 26 - protein, 195 aa, Artificial Sequence
  Synthetic
IVNAKKDVVNTKMFEELKSRLDTLAQEVALLKEQQALQTVSLKRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 27 - protein, 192 aa, Artificial Sequence
  Synthetic
IVNAKKDVVNTKMFEELKSRLDTLAQEVALLKEQQALQTVRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 28 - protein, 191 aa, Artificial Sequence
  Synthetic
IVNAKKDVVNTKMFEELKSRLDTLAQEVALLKEQQALQTRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 29 - protein, 190 aa, Artificial Sequence
  Synthetic
IVNAKKDVVNTKMFEELKSRLDTLAQEVALLKEQQALQRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 30 - protein, 188 aa, Artificial Sequence
  Synthetic
VVNTKMFEELKSRLDTLAQEVALLKEQQALQTVSLKRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 31 - protein, 185 aa, Artificial Sequence
  Synthetic
VVNTKMFEELKSRLDTLAQEVALLKEQQALQTVRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 32 - protein, 184 aa, Artificial Sequence
  Synthetic
VVNTKMFEELKSRLDTLAQEVALLKEQQALQTRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 33 - protein, 183 aa, Artificial Sequence
  Synthetic
VVNTKMFEELKSRLDTLAQEVALLKEQQALQRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 34 - protein, 252 aa, Artificial Sequence [sequence omitted]

SEQ ID NO: 35 - protein, 249 aa, Artificial Sequence
  Synthetic
MVRANKRNEALRIESALLNKIAMLGTEKTAEGGSHHHHHHGSIEPDGGEGPTQKPKKIVNAKKDVVNTKMFEELKSRLDTLAQEVALLKEQQALQTVRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 36 - protein, 248 aa, Artificial Sequence
  Synthetic
MVRANKRNEALRIESALLNKIAMLGTEKTAEGGSHHHHHHGSIEPDGGEGPTQKPKKIVNAKKDVVNTKMFEELKSRLDTLAQEVALLKEQQALQTRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 37 - protein, 247 aa, Artificial Sequence
  Synthetic
MVRANKRNEALRIESALLNKIAMLGTEKTAEGGSHHHHHHGSIEPDGGEGPTQKPKKIVNAKKDVVNTKMFEELKSRLDTLAQEVALLKEQQALQRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 38 - protein, 241 aa, Artificial Sequence
  Synthetic
MVRANKRNEALRIESALLNKIAMLGTEKTAEGGSHHHHHHGSIEPDIVNAKKDVVNTKMFEELKSRLDTLAQEVALLKEQQALQTVSLKRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 39 - protein, 238 aa, Artificial Sequence
  Synthetic
MVRANKRNEALRIESALLNKIAMLGTEKTAEGGSHHHHHHGSIEPDIVNAKKDVVNTKMFEELKSRLDTLAQEVALLKEQQALQTVRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 40 - protein, 237 aa, Artificial Sequence
  Synthetic
MVRANKRNEALRIESALLNKIAMLGTEKTAEGGSHHHHHHGSIEPDIVNAKKDVVNTKMFEELKSRLDTLAQEVALLKEQQALQTRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 41 - protein, 236 aa, Artificial Sequence
  Synthetic
MVRANKRNEALRIESALLNKIAMLGTEKTAEGGSHHHHHHGSIEPDIVNAKKDVVNTKMFEELKSRLDTLAQEVALLKEQQALQRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 42 - protein, 58 aa, Artificial Sequence
  Synthetic
SPGTEPPTQKPKKIVNAKKDVVNTKMFEELKARLDTLSQEVALLKEQQALQTVSLKGS

SEQ ID NO: 43 - protein, 49 aa, Artificial Sequence
  Synthetic
EPPTQKPKKIVNAKKDVVNTKMFEELKSRLDTLAQEVALLKEQQALQTV

SEQ ID NO: 44 - protein, 47 aa, Artificial Sequence
  Synthetic
KPKKIVNAKKDVVNTKMFEELKSRLDTLSQEVALLKEQQALQTVSLK

SEQ ID NO: 45 - protein, 43 aa, Artificial Sequence
  Synthetic
IVNAKKDVVNTKMFEELKSRLDTLSQEVALLKEQQALQTVSLK

SEQ ID NO: 46 - protein, 37 aa, Artificial Sequence
  Synthetic
DVVNTKMFEELKSRLDTLSQEVALLKEQQALQTVSLK

SEQ ID NO: 47 - protein, 33 aa, Artificial Sequence
  Synthetic
TKMFEELKSRLDTLSQEVALLKEQQALQTVSLK

SEQ ID NO: 48 - protein, 29 aa, Artificial Sequence
  Synthetic
EELKSRLDTLSQEVALLKEQQALQTVSLK

SEQ ID NO: 49 - protein, 25 aa, Artificial Sequence
  Synthetic
SRLDTLSQEVALLKEQQALQTVSLK

SEQ ID NO: 50 - protein, 43 aa, Artificial Sequence
  Synthetic
KPKKIVNAKKDVVNTKMFEELKSRLDTLSQEVALLKEQQALQT

SEQ ID NO: 51 - protein, 41 aa, Artificial Sequence
  Synthetic
KPKKIVNAKKDVVNTKMFEELKSRLDTLSQEVALLKEQQAL

SEQ ID NO: 52 - protein, 38 aa, Artificial Sequence
  Synthetic
KPKKIVNAKKDVVNTKMFEELKSRLDTLSQEVALLKEQ

SEQ ID NO: 53 - protein, 34 aa, Artificial Sequence
  Synthetic
KPKKIVNAKKDVVNTKMFEELKSRLDTLSQEVAL

SEQ ID NO: 54 - protein, 31 aa, Artificial Sequence
  Synthetic
KPKKIVNAKKDVVNTKMFEELKSRLDTLSQE

SEQ ID NO: 55 - protein, 40 aa, Artificial Sequence
  Synthetic
IVNAKKDVVNTKMFEELKSRLDTLAQEVALLKEQQALQTV

SEQ ID NO: 56 - protein, 33 aa, Artificial Sequence
  Synthetic
VVNTKMFEELKSRLDTLAQEVALLKEQQALQTV

SEQ ID NO: 57 - protein, 53 aa, Artificial Sequence
  Synthetic
EPPTQKPKKIVNAKKDVVNTKMFEELKSRLDTLAQEVALLKEQQALQTVSLKG

SEQ ID NO: 58 - protein, 52 aa, Artificial Sequence
  Synthetic
EPPTQKPKKIVNAKKDVVNTKMFEELKSRLDTLAQEVALLKEQQALQTVSLK

SEQ ID NO: 59 - protein, 51 aa, Artificial Sequence
  Synthetic
EPPTQKPKKIVNAKKDVVNTKMFEELKSRLDTLAQEVALLKEQQALQTVSL

SEQ ID NO: 60 - protein, 50 aa, Artificial Sequence
  Synthetic
EPPTQKPKKIVNAKKDVVNTKMFEELKSRLDTLAQEVALLKEQQALQTVS

SEQ ID NO: 61 - protein, 49 aa, Artificial Sequence; identical to SEQ ID NO: 43
  Synthetic

SEQ ID NO: 62 - protein, 48 aa, Artificial Sequence
  Synthetic
EPPTQKPKKIVNAKKDVVNTKMFEELKSRLDTLAQEVALLKEQQALQT

SEQ ID NO: 63 - protein, 52 aa, Artificial Sequence
  Synthetic
PPTQKPKKIVNAKKDVVNTKMFEELKSRLDTLAQEVALLKEQQALQTVSLKG

SEQ ID NO: 64 - protein, 48 aa, Artificial Sequence
  Synthetic
PPTQKPKKIVNAKKDVVNTKMFEELKSRLDTLAQEVALLKEQQALQTV

SEQ ID NO: 65 - protein, 51 aa, Artificial Sequence
  Synthetic
PTQKPKKIVNAKKDVVNTKMFEELKSRLDTLAQEVALLKEQQALQTVSLKG

SEQ ID NO: 66 - protein, 50 aa, Artificial Sequence
  Synthetic
TQKPKKIVNAKKDVVNTKMFEELKSRLDTLAQEVALLKEQQALQTVSLKG

SEQ ID NO: 67 - protein, 49 aa, Artificial Sequence
  Synthetic
QKPKKIVNAKKDVVNTKMFEELKSRLDTLAQEVALLKEQQALQTVSLKG

SEQ ID NO: 68 - protein, 48 aa, Artificial Sequence
  Synthetic
KPKKIVNAKKDVVNTKMFEELKSRLDTLAQEVALLKEQQALQTVSLKG

SEQ ID NO: 69 - protein, 47 aa, Artificial Sequence
  Synthetic
PKKIVNAKKDVVNTKMFEELKSRLDTLAQEVALLKEQQALQTVSLKG

SEQ ID NO: 70 - protein, 46 aa, Artificial Sequence
  Synthetic
KKIVNAKKDVVNTKMFEELKSRLDTLAQEVALLKEQQALQTVSLKG

SEQ ID NO: 71 - protein, 45 aa, Artificial Sequence
  Synthetic
KIVNAKKDVVNTKMFEELKSRLDTLAQEVALLKEQQALQTVSLKG

SEQ ID NO: 72 - protein, 44 aa, Artificial Sequence
  Synthetic
IVNAKKDVVNTKMFEELKSRLDTLAQEVALLKEQQALQTVSLKG

SEQ ID NO: 73 - protein, 43 aa, Artificial Sequence
  Synthetic
VNAKKDVVNTKMFEELKSRLDTLAQEVALLKEQQALQTVSLKG

SEQ ID NO: 74 - protein, 42 aa, Artificial Sequence
  Synthetic
NAKKDVVNTKMFEELKSRLDTLAQEVALLKEQQALQTVSLKG

SEQ ID NO: 75 - protein, 41 aa, Artificial Sequence
  Synthetic
AKKDVVNTKMFEELKSRLDTLAQEVALLKEQQALQTVSLKG

SEQ ID NO: 76 - protein, 40 aa, Artificial Sequence
  Synthetic
KKDVVNTKMFEELKSRLDTLAQEVALLKEQQALQTVSLKG

SEQ ID NO: 77 - protein, 39 aa, Artificial Sequence
  Synthetic
KDVVNTKMFEELKSRLDTLAQEVALLKEQQALQTVSLKG

SEQ ID NO: 78 - protein, 37 aa, Artificial Sequence
  Synthetic
VVNTKMFEELKSRLDTLAQEVALLKEQQALQTVSLKG

SEQ ID NO: 79 - protein, 36 aa, Artificial Sequence
  Synthetic
VNTKMFEELKSRLDTLAQEVALLKEQQALQTVSLKG

SEQ ID NO: 80 - protein, 35 aa, Artificial Sequence
  Synthetic
VNTKMFEELKSRLDTLAQEVALLKEQQALQTVSLK

SEQ ID NO: 81 - protein, 34 aa, Artificial Sequence
  Synthetic
NTKMFEELKSRLDTLAQEVALLKEQQALQTVSLK

SEQ ID NO: 82 - protein, 33 aa, Artificial Sequence
  Synthetic
TKMFEELKSRLDTLAQEVALLKEQQALQTVSLK

SEQ ID NO: 83 - protein, 31 aa, Artificial Sequence
  Synthetic
MFEELKSRLDTLAQEVALLKEQQALQTVSLK

SEQ ID NO: 84 - protein, 33 aa, Artificial Sequence; identical to SEQ ID NO: 56
  Synthetic

SEQ ID NO: 85 - protein, 32 aa, Artificial Sequence
  Synthetic
VVNTKMFEELKSRLDTLAQEVALLKEQQALQT

SEQ ID NO: 86 - protein, 30 aa, Artificial Sequence
  Synthetic
VNTKMFEELKSRLDTLAQEVALLKEQQALQ

SEQ ID NO: 87 - protein, 35 aa, Artificial Sequence
  Synthetic
NTKMFEELKSRLDTLAQEVALLKEQQALQTVSLKG

SEQ ID NO: 88 - protein, 34 aa, Artificial Sequence
  Synthetic
TKMFEELKSRLDTLAQEVALLKEQQALQTVSLKG

SEQ ID NO: 89 - protein, 33 aa, Artificial Sequence
  Synthetic
KMFEELKSRLDTLAQEVALLKEQQALQTVSLKG

SEQ ID NO: 90 - protein, 32 aa, Artificial Sequence
  Synthetic
MFEELKSRLDTLAQEVALLKEQQALQTVSLKG

SEQ ID NO: 91 - protein, 52 aa, Artificial Sequence
  Synthetic
EGPTQKPKKIVNAKKDVVNTKMFEELKSRLDTLAQEVALLKEQQALQTVSLK

SEQ ID NO: 92 - protein, 49 aa, Artificial Sequence
  Synthetic
EGPTQKPKKIVNAKKDVVNTKMFEELKSRLDTLAQEVALLKEQQALQTV

SEQ ID NO: 93 - protein, 48 aa, Artificial Sequence
  Synthetic
EGPTQKPKKIVNAKKDVVNTKMFEELKSRLDTLAQEVALLKEQQALQT

SEQ ID NO: 94 - protein, 47 aa, Artificial Sequence
  Synthetic
EGPTQKPKKIVNAKKDVVNTKMFEELKSRLDTLAQEVALLKEQQALQ

SEQ ID NO: 95 - protein, 43 aa, Artificial Sequence
  Synthetic
IVNAKKDVVNTKMFEELKSRLDTLAQEVALLKEQQALQTVSLK

SEQ ID NO: 96 - protein, 40 aa, Artificial Sequence; identical to SEQ ID NO: 55
  Synthetic

SEQ ID NO: 97 - protein, 39 aa, Artificial Sequence
  Synthetic
IVNAKKDVVNTKMFEELKSRLDTLAQEVALLKEQQALQT

SEQ ID NO: 98 - protein, 38 aa, Artificial Sequence
  Synthetic
IVNAKKDVVNTKMFEELKSRLDTLAQEVALLKEQQALQ

SEQ ID NO: 99 - protein, 35 aa, Artificial Sequence; identical to SEQ ID NO: 80
  Synthetic

SEQ ID NO: 100 - protein, 32 aa, Artificial Sequence
  Synthetic
VNTKMFEELKSRLDTLAQEVALLKEQQALQTV

SEQ ID NO: 101 - protein, 31 aa, Artificial Sequence
  Synthetic
VNTKMFEELKSRLDTLAQEVALLKEQQALQT

SEQ ID NO: 102 - protein, 30 aa, Artificial Sequence; identical to SEQ ID NO: 86
  Synthetic

SEQ ID NO: 103 - protein, 40 aa, Artificial Sequence
  Synthetic
MIVNAKKDVVNTKMFEELKSRLDTLAQEVALLKEQQALQT

SEQ ID NO: 104 - protein, 32 aa, Artificial Sequence
  Synthetic
MVNTKMFEELKSRLDTLAQEVALLKEQQALQT

SEQ ID NO: 105 - protein, 53 aa, Artificial Sequence
  synthetic
EPPTQKPKKIVNAKKDVVNTKMFEELKARLDTLSQEVALLKEQQALQTVSLKG

SEQ ID NO: 106 - protein, 52 aa, Artificial Sequence
  Synthetic
EPPTQKPKKIVNAKKDVVNTKMFEELKARLDTLSQEVALLKEQQALQTVSLK

SEQ ID NO: 107 - protein, 51 aa, Artificial Sequence
  Synthetic
EPPTQKPKKIVNAKKDVVNTKMFEELKARLDTLSQEVALLKEQQALQTVSL

SEQ ID NO: 108 - protein, 50 aa, Artificial Sequence
  Synthetic
EPPTQKPKKIVNAKKDVVNTKMFEELKARLDTLSQEVALLKEQQALQTVS

SEQ ID NO: 109 - protein, 49 aa, Artificial Sequence
  Synthetic
EPPTQKPKKIVNAKKDVVNTKMFEELKARLDTLSQEVALLKEQQALQTV

SEQ ID NO: 110 - protein, 52 aa, Artificial Sequence
  Synthetic
PPTQKPKKIVNAKKDVVNTKMFEELKARLDTLSQEVALLKEQQALQTVSLKG

SEQ ID NO: 111 - protein, 51 aa, Artificial Sequence
  Synthetic
PTQKPKKIVNAKKDVVNTKMFEELKARLDTLSQEVALLKEQQALQTVSLKG

SEQ ID NO: 112 - protein, 50 aa, Artificial Sequence
  Synthetic
TQKPKKIVNAKKDVVNTKMFEELKARLDTLSQEVALLKEQQALQTVSLKG

SEQ ID NO: 113 - protein, 49 aa, Artificial Sequence
  Synthetic
QKPKKIVNAKKDVVNTKMFEELKARLDTLSQEVALLKEQQALQTVSLKG

SEQ ID NO: 114 - protein, 48 aa, Artificial Sequence
  Synthetic
KPKKIVNAKKDVVNTKMFEELKARLDTLSQEVALLKEQQALQTVSLKG

SEQ ID NO: 115 - protein, 47 aa, Artificial Sequence
  Synthetic
PKKIVNAKKDVVNTKMFEELKARLDTLSQEVALLKEQQALQTVSLKG

SEQ ID NO: 116 - protein, 46 aa, Artificial Sequence
  Synthetic
KKIVNAKKDVVNTKMFEELKARLDTLSQEVALLKEQQALQTVSLKG

SEQ ID NO: 117 - protein, 45 aa, Artificial Sequence
  Synthetic
KIVNAKKDVVNTKMFEELKARLDTLSQEVALLKEQQALQTVSLKG

SEQ ID NO: 118 - protein, 44 aa, Artificial Sequence
  Synthetic
IVNAKKDVVNTKMFEELKARLDTLSQEVALLKEQQALQTVSLKG

SEQ ID NO: 119 - protein, 43 aa, Artificial Sequence
  Synthetic
VNAKKDVVNTKMFEELKARLDTLSQEVALLKEQQALQTVSLKG

SEQ ID NO: 120 - protein, 42 aa, Artificial Sequence
  Synthetic
NAKKDVVNTKMFEELKARLDTLSQEVALLKEQQALQTVSLKG

SEQ ID NO: 121 - protein, 41 aa, Artificial Sequence
  Synthetic
AKKDVVNTKMFEELKARLDTLSQEVALLKEQQALQTVSLKG

SEQ ID NO: 122 - protein, 40 aa, Artificial Sequence
  Synthetic
KKDVVNTKMFEELKARLDTLSQEVALLKEQQALQTVSLKG

SEQ ID NO: 123 - protein, 39 aa, Artificial Sequence
  Synthetic
KDVVNTKMFEELKARLDTLSQEVALLKEQQALQTVSLKG

SEQ ID NO: 124 - protein, 37 aa, Artificial Sequence
  Synthetic
VVNTKMFEELKARLDTLSQEVALLKEQQALQTVSLKG

SEQ ID NO: 125 - protein, 36 aa, Artificial Sequence
  Synthetic
VNTKMFEELKARLDTLSQEVALLKEQQALQTVSLKG

SEQ ID NO: 126 - protein, 35 aa, Artificial Sequence
  Synthetic
VNTKMFEELKARLDTLSQEVALLKEQQALQTVSLK

SEQ ID NO: 127 - protein, 34 aa, Artificial Sequence
  Synthetic
NTKMFEELKARLDTLSQEVALLKEQQALQTVSLK

SEQ ID NO: 128 - protein, 33 aa, Artificial Sequence
  Synthetic
TKMFEELKARLDTLSQEVALLKEQQALQTVSLK

SEQ ID NO: 129 - protein, 31 aa, Artificial Sequence
  Synthetic
MFEELKARLDTLSQEVALLKEQQALQTVSLK

SEQ ID NO: 130 - protein, 71 aa, Artificial Sequence
  Synthetic
MGSHHHHHGSIQGRSPGTEPPTQKPKKIVNAKKDVVNTKMFEELKSRLDTLAQEVALLKEQQALQTVSLKG

(19) United States
(12) Patent Application Publication
(10) Pub. No.: US 2010/0028995 A1
(43) Pub. Date: Feb. 4, 2010

(54) Tetranectin Trimerizing Polypeptides

(75) Inventors: Jonas Heilskov Graversen, Hornslet (DK); Hans Christian Thøgersen, Sabro (DK); Anke Kretz-Rommel, San Diego, CA (US); Michael Etzerodt, Hinnerup (DK); Thor Las Holtet, Ronde (DK); Mikkel Holmen Andersen, Sporup (DK); John Nieland, Arhus C (DK)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Anaphore, Inc.

(21) Appl. No.: 12/420,680

(22) Filed: Apr. 8, 2009

Related U.S. Application Data
(63) Continuation-in-part of application No. 12/015,493, filed on Jan. 16, 2008.
Application No. 12/015,493 is a continuation of application No. 11/064,115, filed on Feb. 23, 2005, now abandoned.
Application No. 11/064,115 is a continuation-in-part of application No. 12/247,897, filed on Oct. 8, 2008.
(60) Provisional application No. 60/546,200, filed on Feb. 23, 2004.
(60) Provisional application No. 60/978,254, filed on Oct. 8, 2007.

(30) Foreign Application Priority Data
Feb. 23, 2004 (DK) PA 2004 00283
Feb. 23, 2005 (DK) PCT/DK05/00121

(51) Int. Cl. C12N 5/00; C07K 14/00; C12N 15/11; C12N 15/00; A61K 38/16

(52) U.S. Cl. 435/325; 530/324; 530/350; 536/23.1; 435/320.1; 514/12

(57) ABSTRACT

Tetranectin trimerizing polypeptides and fusion proteins including the polypeptides and therapeutic polypeptides and proteins. Trimeric complexes of the polypeptides and fusion proteins. Pharmaceutical compositions of the polypeptides, fusion proteins and the trimeric complexes.

### CROSS-REFERENCE TO RELATED APPLICATION

This application is a continuation-in-part of U.S. patent application Ser. No. 12/015,493, filed Jan. 16, 2008, which is a continuation of U.S. patent application Ser. No. 11/064,115, filed Feb. 23, 2005, and this application is a continuation-in-part of U.S. patent application Ser. No. 12/247,897, filed Oct. 8, 2008. U.S. Ser. No. 11/064,115 claims the benefit of U.S. Provisional Application Ser. No. 60/546,200, filed Feb. 23, 2004. U.S. Ser. No. 12/247,897 claims the benefit of U.S. Provisional Patent Application Ser. No. 60/978,254, filed Oct. 8, 2007. Each of the above-referenced applications are incorporated by reference herein in their entirety.

### SEQUENCE LISTING STATEMENT

The sequence listing is filed in this application in electronic format only and is incorporated by reference herein. The sequence listing text file “09-226.5T25.txt” was created on Apr. 8, 2009, and is 100,303 bytes in size.

### FIELD OF THE INVENTION

The invention relates to treatment of diseases with therapeutic polypeptides. More particularly, the invention relates to the treatment of diseases with fusion proteins and with polypeptides that include a therapeutic polypeptide and a polypeptide that is able to form a trimer.

### BACKGROUND OF THE INVENTION

Treatment of diseases with therapeutic peptides has been gaining momentum over the last decade. A variety of peptides are now available to the clinician, and hundreds more are being developed. While the technology has proved effective and promising, a number of challenges associated with therapeutic peptides remain, including in particular potency, serum stability, half-life, avidity, production challenges and, in some cases, unwanted immune response.

Tetranectin (TN) is a Ca²⁺-binding trimeric C-type lectin which is present in blood plasma and in the extracellular matrix of certain tissues. The tetranectin group of proteins includes, for example, tetranectin isolated from man (Swissprot P05452) (SEQ ID NO:1), mouse (Swissprot P43025) (SEQ ID NO:2), chicken (Swissprot Q9DDD4) (SEQ ID NO:3), bovine (Swissprot Q2KIS7) (SEQ ID NO:4), Atlantic salmon (Swissprot B5XCV4) (SEQ ID NO:5), frog (Swissprot Q5I0R9) (SEQ ID NO:6), zebrafish (GenBank XP_{—}701303) (SEQ ID NO:7) and the highly related C-type lectin homologues isolated from the cartilage of cattle (Swissprot u22298) (SEQ ID NO:8) and from reef shark (Swissprot p26258) (SEQ ID NO:9).

The mature human tetranectin polypeptide chain of 181 amino acid residues is encoded in three exons as shown by molecular cloning and characterization of the gene (SEQ ID NO:1). Exon 3 of the human tetranectin gene encodes a separate functional and structural unit, a single long-form so-called carbohydrate recognition domain (CRD), with three intra-chain-disulphide bridges. The tetranectin CRD is considered to belong to a distinct class of C-type lectins related to C-type lectins by sequence homology, conservation of disulphide topology and by the presence of an almost conserved suit of amino acid residues known to be involved in binding of calcium ions.

Tetranectin was first identified as a plasma protein binding to the kringle-4 domain of plasminogen. The site in tetranectin involved in binding to plasminogen resides entirely in the CRD-domain (encoded by exon 3). Binding is calcium sensitive, the kringle-4 binding site in tetranectin overlaps the putative carbohydrate binding site of the CRD domain. Accordingly, TN exons 1 and 2, i.e. the trimerisation unit in TN, do not exhibit any plasminogen-binding affinity. Tetranectin has also been reported to bind to sulfated polysaccharides like heparin. Binding specificity to heparin and N-acetylglucosamines resides in the trimerization unit with residues 6-15, and particularly K9 being most important.

Therapeutic peptides including a tetranectin trimerizing domain have been described, e.g., U.S. Patent Publication Nos. 2007/0154901, 2004/0132094, 2007/0275393, 2007/0010658, and 2006/0199251, each of which is incorporated by reference in its entirety.

### BRIEF DESCRIPTION OF THE DRAWINGS

FIG. 1 shows alignment of the amino acid sequences of the trimerizing structural element of the tetranectin protein family. Amino acid sequences (one letter code) corresponding to residue E1 to K52 comprising exon 1, exon 2 and the first three residues of exon 3 of human tetranectin (SEQ ID NO: 10). Sequences include murine tetranectin (SEQ ID NO: 11); chicken (SEQ ID NO:12), bovine (SEQ ID NO:13), Atlantic salmon (SEQ ID NO:14), frog (SEQ ID NO:15), zebrafish (SEQ ID NO:16) tetranectin homologous protein isolated from reefshark cartilage (SEQ ID NO:17) and tetranectin homologous protein isolated from bovine cartilage (SEQ ID NO:18). Residues at a and d positions in the heptad repeats are listed in boldface. The listed consensus sequence (SEQ ID NO:19) of the tetranectin protein family trimerising structural element comprise the residues present at a and d positions in the heptad repeats shown in the figure in addition to the other conserved residues of the region. “*” denotes an aliphatic hydrophobic residue. Residues corresponding to exon 2 and the first three residues of exon 3 of human tetranectin (V17-K52) are underlined.

FIG. 2 shows the results of CII-H6-GrB-TripK-IL-1Ra refolding by dialysis.

FIG. 3 displays the capturing CII-H6-GrB-TripK-IL-1Ra on NiNTA.

FIG. 4 is a graph showing the ability of GG-TripV-IL-1Ra (Trip V-IL-1Ra), GG-TripK-IL-1Ra (Trip K-IL-1Ra), GG-TripT-IL-1Ra (Trip T-IL-1Ra) and GG-TripT-IL-1Ra (Trip T-IL-1Ra) to inhibit IL-1 induction of IL-8 in U937 cells.

FIG. 5 is a graph showing the ability of pegylated TripT and TripV to inhibit IL-1 induction of IL-8 in U937 cells as compared to non-pegylated forms and KINERET®.

FIG. 6 is a graph showing the ability of TripT-IL-1Ra, I10-TripT-IL-1Ra, V17-TripT-IL-1Ra used in the PK study to inhibit IL-1 induction of IL-8 in U937 cells

FIG. 7 is a graph showing the blood concentrations of TripT-IL-1Ra, 110-TripT-IL-1Ra, and V17-TripT-IL-1Ra after 100 mg/kg i.v. injection in rats.

FIG. 8 shows an SDS-PAGE analysis of multiple batches of Met-I10-TrpT-IL-1Ra (LM022 and LM023) and GG-V17-TrpT-IL-1Ra (CF019 and CF020) protein yields.

FIG. 9 shows analytical SEC results of Met-I10-TrpT-IL-1Ra and GG-V17-TrpT-IL-1Ra protein yields.

FIG. 10 shows the results of the rat CIA study. Ankle diameters of female Lewis rats with type II collagen arthritis were measured following treatment with Vehicle (10 mM phosphate buffer pH 7.4), or equimolar amounts of IL-1ra administering either monomeric IL-1ra (100 mg/kg KINERET®), or trimerized IL1ra (120 mg/kg Met-I10-TripT-IL1ra, or 120 mg/kg GG-V17-TripT-IL1ra).

FIG. 11 is a graph showing the reduction of final paw weight when rats treated with KINERET®, Met-I10-TripT-IL1ra QD, or GG-V17-TripT-IL1ra QD, as compared to vehicle treated disease control animals.

FIG. 12 is a graph showing the of blood glucose levels observed after daily i.p. dosing of either I10-TripT-IL1-Ra or KINERET®.

FIG. 13 is graph showing the fitting of a differential scanning calorimetry (DSC) scan of 0.5 mM Trip-A to the non-2-state model

FIG. 14 is a graph showing the fitting of a DSC scan of 0.5 mM Trip-A to the dissociation with dCp model

FIG. 15 is a series of DSC scans shows the unfolding and refolding of Trip-A at 0.5 mM.

FIGS. 16A-D show consecutive up and down DSC scans of Trip A. FIGS. 16A and 16C are up scans, FIGS. 16B and 16D down scans.

FIGS. 17A and 17B show a DSC scan and fits at pH 4 (17A) and 3 (17B).

FIGS. 18A-18H show representative DSC scans for N-terminal deletion peptides.

FIGS. 19A-19E show representative DSC scans for C-terminal deletion peptides.

FIG. 20 shows a DSC scan for a tetranectin polypeptide (trimeric Met I-10-TripT) having both an N-terminal and a C-terminal truncation.

FIG. 21 shows a DSC scan for a tetranectin polypeptide (Met V-17-TripT) having both an N-terminal and a C-terminal truncation.

FIG. 22 shows examples of tetranectin trimerizing module truncations of the invention.

FIGS. 23A, B and C show examples of tetranectin trimerizing module truncations of the invention.

### SUMMARY OF THE INVENTION

In one aspect, the invention is directed to an isolated polypeptide having an amino acid sequence of VNTKMFEELKSRLDTLAQEVALLKEQQALQTVSLK (SEQ ID NO:80) and having:
(a) an amino-terminal truncation of 0 to 6 amino acid residues, and wherein three polypeptides form a trimeric complex;
(b) a carboxyl-terminal truncation of 0 to 15 amino acid residues, and wherein three polypeptides form a trimeric complex; or
(c) an amino-terminal truncation of 0 to 6 amino acid residues and a carboxyl-terminal truncation of 0 to 15 residues, and wherein three polypeptides form a trimeric complex.

In this aspect, the isolated polypeptide may have an N terminus is T20 or a C-terminus of one of K52, V49, T48, Q47, L46, and Q43.

In another aspect, the invention is directed to isolated polypeptide having an amino acid sequence of EPPTQKPKKIVNAKKDVVNTKMFEELKSRLDTLAQEVALLKEQQALQT (SEQ ID NO:62) and having:
(a) an amino-terminal truncation of 0 to 23 amino acid residues, and wherein three polypeptides form a trimeric complex;
(b) a carboxyl-terminal truncation of 0 to 12 amino acid residues, and wherein three polypeptides form a trimeric complex; or
(c) an amino-terminal truncation of 0 to 23 amino acid residues and a carboxyl-terminal truncation of 0 to 12 residues, and wherein three polypeptides form a trimeric complex.

In this aspect, the isolated polypeptide may have an N terminus is selected from one of K6, I10, D16, V17, and T20, and may have a C-terminus selected from one of T48, Q47, L46, and Q43.

In another aspect, the invention is directed to an isolated polypeptide having an amino acid sequence of PPTQKPKKIVNAKKDVVNTKMFEELKSRLDTLAQEVALLKEQQALQTV (SEQ ID NO:64) and having:
(d) an amino-terminal truncation of 0 to 22 amino acid residues, and wherein three polypeptides form a trimeric complex;
(e) a carboxyl-terminal truncation of 0 to 13 amino acid residues, and wherein three polypeptides form a trimeric complex; or
(f) an amino-terminal truncation of 0 to 22 amino acid residues and a carboxyl-terminal truncation of 0 to 13 residues, and wherein three polypeptides form a trimeric complex.

In this aspect, the isolated polypeptide may have an N terminus of one of K6, I10, D16, V17, and T20, and C-terminus of one of V49, T48, Q47, L46, and Q43.

In a further aspect of the invention, the isolated polypeptide may include comprising an amino acid sequence that is at least 50%, 60%, 70%, or more identical to any of the foregoing polypeptides

Still further, in one aspect the invention is directed to a trimeric polypeptide complex comprising three of the polypeptides of the invention.

In another aspect, the invention is directed to a fusion protein including a polypeptide of the invention and a therapeutic peptide or polypeptide. The fusion proteins may form a trimeric complex.

The invention is also directed to isolated polynucleotides, vectors and host cells that are useful for producing the polypeptides and fusion proteins of the invention.

Even further, the invention is directed to pharmaceutical compositions including the polyepeptides, fusion proteins or trimeric complexes of the invention.

### DETAILED DESCRIPTION OF VARIOUS EMBODIMENTS THE INVENTION

The invention is directed to compounds and methods for treating diseases. In one aspect, the invention is directed to a fusion protein including a therapeutic polypeptide sequence and a polypeptide sequence of a trimerizing domain. Three fusion proteins may trimerize to form a trimeric complex having therapeutic properties. The trimeric complex provides for greater stability and improved pharmacokinetic and pharmacodynamic properties than the therapeutic peptide alone, and provides a favorable safety profile.

The polypeptides of the invention can trimerize to form a highly stable homotrimeric and heterotrimeric complexes when attached by a peptide bond or suitable linker at either or at both termini to other proteins. Accordingly, heterologous polypeptide sequences may be placed amino-terminally or carboxy-terminally to the trimerising polypeptides allowing for the formation of one molecular assembly containing up to six copies of one particular polypeptide sequence or functional entities, or the formation of one molecular assembly containing up to six different polypeptide sequences, each contributing one or more functional properties.

In one aspect, the invention is directed to portion of a polypeptide molecule of the tetranectin family which is responsible for trimerization between monomers of the tetranectin polypeptide. The term is also intended to embrace truncations and variants of a naturally occurring tetranectin family member. Variants include naturally occurring polypeptides that have been modified in the amino acid sequence. Truncations include natural trimerizing sequences that have deletions of amino acid residues at the N-terminus, the C-terminus or both. The truncated polypeptides of the invention may include variants. The truncation, variants, or both should not adversely affect, to any substantial degree, the trimerization properties relative to those of the native tetranectin family member molecule. In various aspects of the invention, the polypeptides are derived from human tetranectin, murine tetranectin, bovine tetranectin, Atlantic salmon tetranectin, chicken tetranectin, C-type lectin of bovine cartilage, or C-type lectin of shark cartilage.

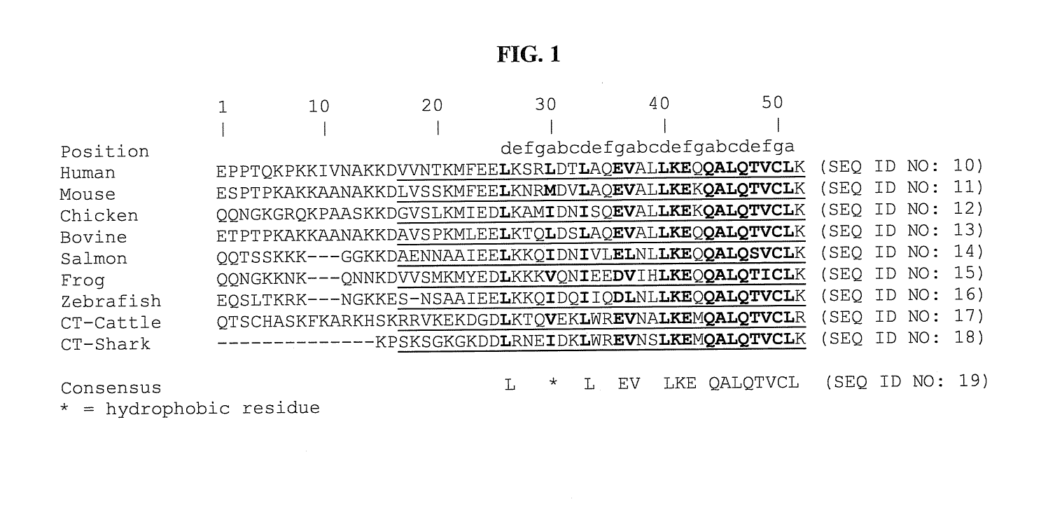

The 49 residue polypeptide sequence encoded by exons 1 and 2 of tetranectin appears to be unique to the tetranectin group of proteins (see FIG. 1) as no significant sequence homology to other known polypeptide sequences has been established. From the combination of sequence and structure data it becomes clear that trimerization in tetranectin is in fact generated by a structural element (FIG. 1), comprising the amino acid residues encoded by exon two and the first three residues of exon 3 by an unusual heptad repeat sequence. This amino acid sequence is characterized by two copies of heptad repeats (abcdefg) with hydrophobic residues at a and d positions as are other alpha helical coiled coils. These two heptad repeats are in sequence followed by an unusual third copy of the heptad repeat, where glutamine 44 and glutamine 47 not only substitute the hydrophobic residues at both the a and d position, but are directly involved in the formation of the triple alpha helical coiled coil structure. These heptad repeats are additionally flanked by two half-repeats with hydrophobic residues at the d and a position, respectively. The presence of beta-branched hydrophobic residues at a or d positions in alpha helical coiled coil are kown to influence the state of oligomerization. In the tetranectin structural element only one conserved valine (V37) is present. At sequence position 29 in tetranectin no particular aliphatic residue appears to be preferred.

It is apparent that the triple-stranded, coiled-coil structure in tetranectin to a large extent is governed by interactions that are unexpected in relation to those characteristic among the group of known coiled coil proteins. The polypeptides of the invention form a stable trimeric molecule. A substantial part of the polypeptide exists in the oligomeric state of and can be cross-linked as trimeric molecules even at 70° C. The exchange of monomers between different trimers can only be detected after exposure to elevated temperature is evidence of an extremely high stability of the tetranectin trimerising polypeptides of the invention. This feature must be reflected in the amino acid sequence of the structural element. In particular, the presence and position of the glutamine containing repeat in the sequential array of heptad repeats is, together with the presence and relative position of the other conserved residues in the consensus sequence (FIG. 1), considered important for the formation of these stable trimeric molecules.

Accordingly, in various aspects of the invention, a fusion protein contains an amino acid sequence—a trimerizing domain—which forms a trimeric complex with two other trimerizing domains. A trimerizing domain can associate with other trimerizing domains of identical amino acid sequence (a homotrimer), or with trimerizing domains of different amino acid sequence (a heterotrimer). Such an interaction may be caused by covalent bonds between the components of the trimerizing domains as well as by hydrogen bond forces, hydrophobic forces, van der Waals forces and salt bridges.

Truncated tetranectin trimerizing polypeptides of the invention include at least the sequence EELKSRLDTLAQEV (SEQ ID NO:20), which represents amino acid residues 24-36 of SEQ ID NO:10. The trimerizing polypeptides, therefore, are at least 14 residues long. In other aspects of the invention, the polypeptides are 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids long. In addition, the truncated polypeptides embraces variants of a naturally occurring member of the tetranectin family of proteins, and in particular variants that have been modified in the amino acid sequence without adversely affecting, to any substantial degree, the ability of the trimerizing domain to form alpha helical coiled coil trimers. FIG. 1 shows an alignment of several species of tetranectin polypeptides, and shows a consensus sequence of a tetranectin trimerizing polypeptide. Polypeptides of the invention exhibit sequence homology of at least 50%, at least 55%, at least 60%, at least 65%, at least 70%, at least 75%, at least 80%, at least 85%, at least 90% or at least 95% to the corresponding region of SEQ ID NO:10. In one aspect, the substitution is a result of conservative substitution. For the purposes of the invention, percent identify between sequences is determined by comparing the truncated tetranectin sequences to the corresponding portion of SEQ ID NO:10. Insertions, deletions or substitutions within the sequence between the termini are considered in the determination. N-terminal and C-terminal residues absent as a result of the truncations are not considered.

When present in the polypeptides of the invention, the cysteine residue 50 can, when desirable, be mutagenized to serine, threonine, methionine or to any other amino acid residue in order to avoid formation of an unwanted inter-chain disulphide bridge, which eventually would lead to uncontrolled multimerisation, aggregation and precipitation of a polypeptide product harboring this sequence unless there is a corresponding cys forming disulfide bridges as is the case for C-type lectin domains. Also, residue number 28 in native hTN is polymorphic, being either a Serine or an Alanine. Other known variants include at least one amino acid residue selected from amino acid residue nos. 6, 21, 22, 24, 25, 27, 28, 31, 32, 35, 39, 41, and 42 (numbering according to SEQ ID NO:10), which may be substituted by any non-helix breaking amino acid residue. These residues have been shown not to be directly involved in the intermolecular interactions that stabilize the trimeric complex between three TTSEs of native tetranectin monomers. In one aspect shown in FIG. 1, the TTSE has a repeated heptad having the formula a-b-c-d-e-f-g (N to C), wherein residues a and d (i.e., positions 26, 33, 37, 40, 44, 47, and 51 may be any hydrophobic amino acid (numbering according to SEQ ID NO:10).

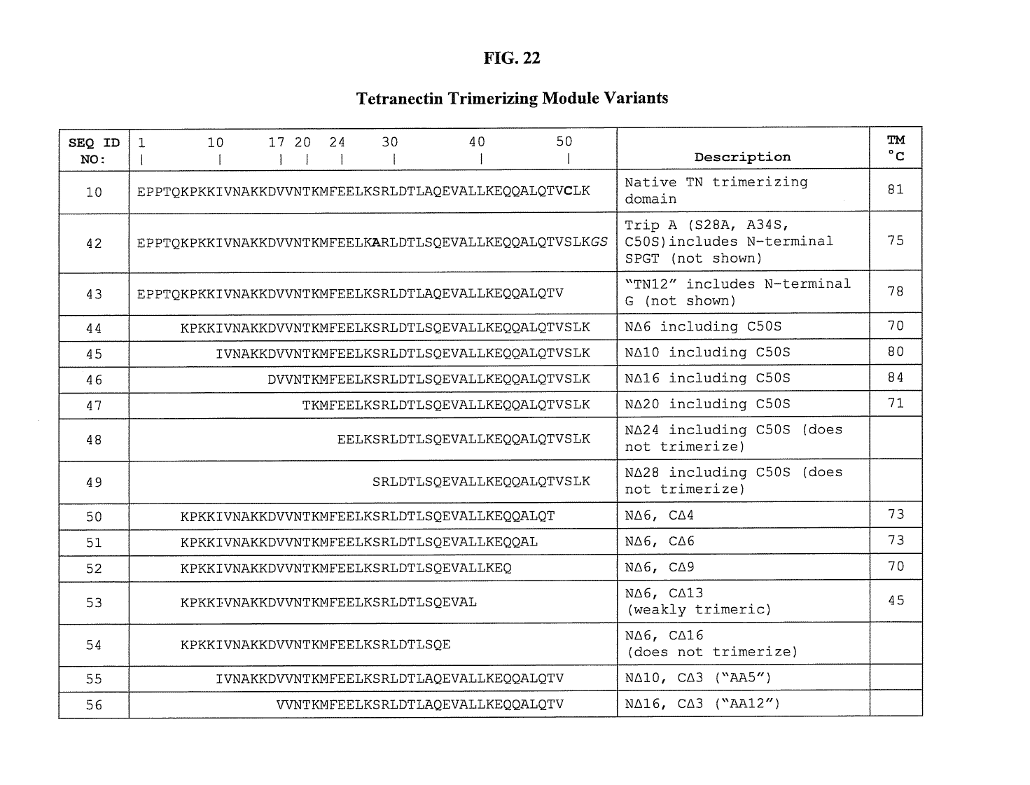

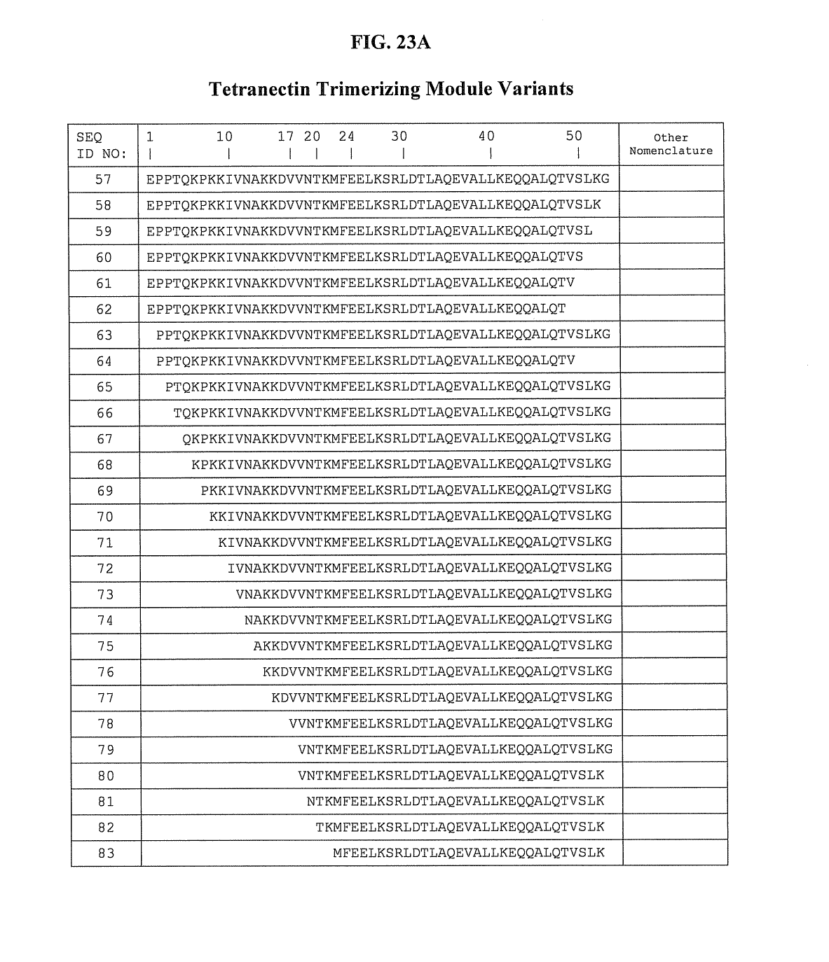

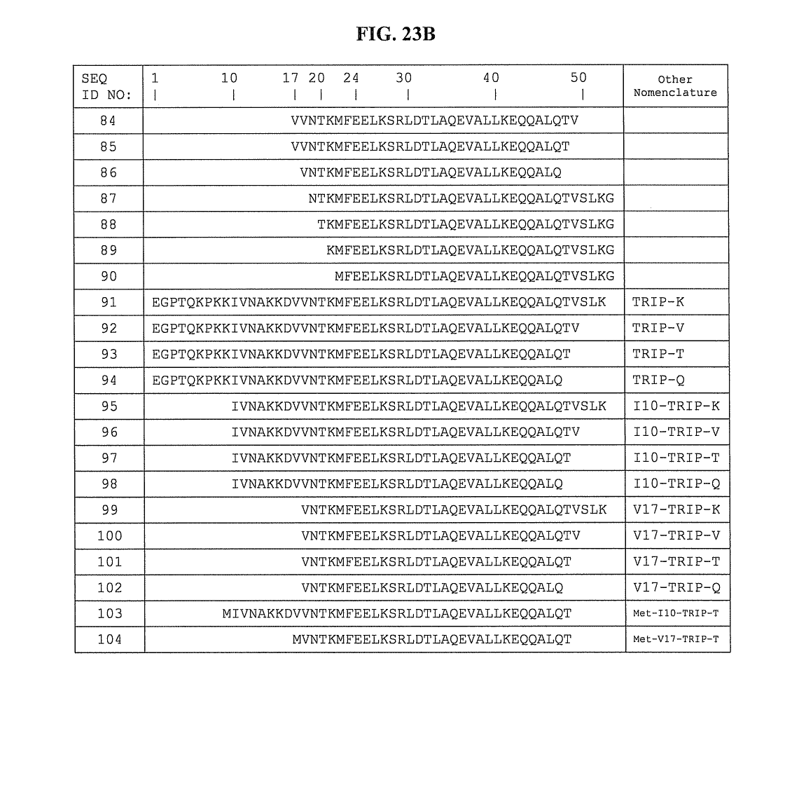

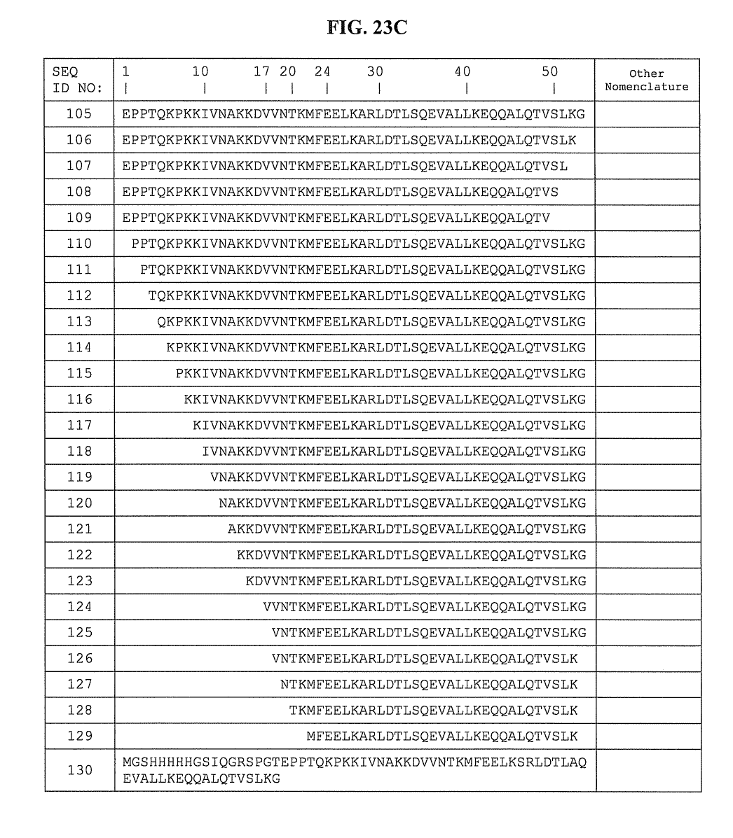

In various aspects of the invention, the trimerizing module includes truncated N-terminal and/or C-terminal truncated forms of the polypeptide of SEQ ID NO:10. For example, the N-terminus is any of residues 1-24 of SEQ ID NO:10, and the C-terminus is any one of residues 37-52 of SEQ ID NO:10. Accordingly, in various aspects of the invention, the N terminus is one of E1, P2, P3, T4, Q5, K6, P7, K8, K9, I10, V11, N12, A13, K14, K15, D16, V17, V18, N19, T20, K21, M22, F23, E24. In other aspects of the invention, the C terminus is V35, A36, L37, L38, K39, K40, E41, Q42, Q43, Q44, A45, L46, Q47, T48, V49, C50, L51 and K52. In further aspects of the invention, both the N and C terminus can be truncated as described herein. Particular examples include truncated forms of SEQ ID NO:1 include peptides having an N-terminus of E1, K6, I10, D16, V17, and T20. In addition, particular examples of C-terminal truncations include peptides have a C-terminus of V49, T48, Q44 and L39. Examples of a number of truncated polypeptides of the invention are shown in FIGS. 22 and 23A-C. In each of these examples, the cysteine at position 50 has been changed to serine as described herein. Also, in many of the sequences, the native proline residue at position 2 has been changed to glycine to assist in purification. SEQ ID NOS 105-129, among others, include S28A and A34S substitutions.

Accordingly, in one aspect, the invention is directed to a truncated form of isolated polypeptide having an amino acid sequence of VNTIMFEELKSRLDTLAQEVALLKEQQALQTVCLK (SEQ ID NO:80). The polypeptide has an amino-terminal truncation of 0 to 6 amino acid residues, a carboxyl-terminal truncation of 0 to 15 amino acid residues; or an amino-terminal truncation of 0 to 6 amino acid residues and a carboxyl-terminal truncation of 0 to 15 residues, and wherein three polypeptides form a trimeric complex. In this aspect of the invention, the N-terminus is, for example, T19. The C-terminus is, for example, V49, T48, Q47, L46, and Q43. In each of these embodiments, the truncated polypeptides are capable of forming a trimeric complexes. In a particular embodiment, the truncation at the C-terminus is at least 1 residue.

As another example, the invention is directed to a truncated form of an isolated polypeptide having an amino acid sequence of EPPTQKKIVNAKKDVVNTKMFEELKSRLDTLAQEVALLKEQQALQT (SEQ ID NO:62). The polypeptide has an amino-terminal truncation of 0 to 23 amino acid residues, a carboxyl-terminal truncation of 0 to 12 amino acid residues, or an amino-terminal truncation of 0 to 23 amino acid residues and a carboxyl-terminal truncation 0 to 12 residues, and wherein three polypeptides form a trimeric complex. In this aspect of the invention, the N-terminus is selected from, for example, K6, I10, D16, V17, and T19, and the C-terminus is selected from, for example, V49, T48, Q47, L46, and Q43. In each of these embodiments, the truncated polypeptides are capable of forming a trimeric complex.

The invention is also directed to an isolated polypeptide comprising an amino acid sequence of PPTQKPKKIVNAKKDVVNTKMFEELKSRLDTLAQEVALLKEQQALQTV (SEQ ID NO:64) having an amino-terminal truncation of 0 to 22 amino acid residues; a carboxyl-terminal truncation of 0 to 13 amino acid residues, an amino-terminal truncation of 0 to 22 amino acid residues and a carboxyl-terminal truncation of 0 to 13 residues. In this aspect of the invention, the N terminus is selected from, for example, K6, I10, D16, V17, and T19, and the C terminus is selected from, for example, V49, T48, Q47, L46, and Q43. In each of these embodiments, the truncated polypeptides are capable of forming a trimeric complexes.

In another aspect of the invention, the isolated polypeptides, fusion proteins and/or trimeric complexes of the invention do not include an any one or more of SEQ ID NOS: 58-61, 63, 65-79, 81-83 and 105-130.

The isolated polypeptides of the invention are truncated forms of longer tetranectin polypeptides, and may further comprise heterologous polypeptide sequences. When the isolated polypeptides comprise the truncated forms of the tetranectin polypeptides, it is to be understood that the truncated forms do not comprise a naturally occurring tetranectin sequence, or variant thereof, that would otherwise be present beyond the N or C terminus of the truncated polypeptides.

In one aspect, the N-terminus of the truncated polypeptide is one of residues E1, P2, P3, and T4. Therefore, the polypeptide includes residue T4, which is the glycosylation site for the polypeptide. When expressed in a mammalian cell, the polypeptide will be glycosylated, which provides for binding to n-acetylglucosamines (e.g., heparin) and potentially longer half-life of the molecule. This might be particularly advantageous for local injections to sites with high N-acetylglucosamine expression such as cartilage. In some aspects when glycosylation is not desirable, the N-terminus of the polypeptide is a residue other than E1, P2, P3, and T4, e.g., one of residues Q5-F23, which results in a polypeptide without the T4 glycosylation site. In other aspects, the polypeptide includes a residue at position 4 other than threonine and that is does not provide a glycosylation site. Also, to prevent glycosylation, a polypeptide including T4 may be expressed in a system that does not glycosylate the polypeptide.

In further embodiments, the tetranectin trimerization domain may be modified by the incorporation of polyhistidine sequence and/or a protease cleavage site, e.g, Blood Coagulating Factor Xa or Granzyme B (see US 2006/0199251, which is incorporated herein by reference), and by including a C-terminal KG or KGS sequence. Also, to assist in purification, proline at position 2 may be substituted with glycine.

The polypeptides of the invention may be used for the preparation of therapeutic compositions for use in the treatment diseases. In this aspect a truncation tetranectin trimerizing polypeptide is fused to heterologous polypeptide sequences may be placed amino-terminally or carboxy-terminally to the trimerising polypeptides. Trimerization results in the formation of one molecular assembly containing several copies (e.g., six copies) of one particular polypeptide sequence or functional entities, or the formation of one molecular assembly containing one or several different polypeptide sequences, each contributing individual or multiple functional properties.

For example, the same heterologous polypeptide can be attached to the N-terminus and/or the C-terminus of one or more of the trimerizing polypeptides. In this aspect, therefore, up to six copies of a single polypeptide, such as a therapeutic polypeptide, can be associated with a complex including the trimerizing polypeptides and the therapeutic polypeptides. In another example, different polypeptides can be attached to the N-terminus and C-terminus of the trimerizing polypeptides. Each of these polypeptides can contribute one or more different functional properties. For example, a trimerized polypeptide complex can include a therapeutic polypeptide associated with the N-terminus of each of the individual trimerizing polypeptides, and a polypeptide useful for purification of the complex associated with the C-termini (or vice versa). In another example, one or more of the heterologous polypeptides associated with the trimeric complex can be a string of polypeptides having multiple functions. For example, a string of apoptosis-inducing peptides such as KLAIK and L-V131K separated by proteosomal cleavage sites such as RR can be fused to the either the N-terminus or the C-terminus of a trimerizing polypeptide of a trimeric complex. Upon internalization in a cell, the apoptosis-inducing peptides are released and trigger apoptosis. See Mi, et al, Mol Ther (2003) 8:295-305; and Chen, et al., Antimicrob Agents Chemother (2007) 51:1398-1406. Furthermore, for vaccine development, a string of epitopes derived from tumor associated antigens such as HER-2, tyrosinase, etc. or viral coat antigens can be attached.

In one aspect, the therapeutic polypeptides for fusion with the trimerizing polypeptides of the invention are natural or non-natural sequences that are agonists or antagonists for receptors that mediate a biological function. In another aspect, a polypeptide that specifically binds to a receptor is contained in the loop region of the CRD of a tetranectin. The polypeptide may be a natural polypeptide, or may be a fragment thereof, or a sequence that is not a naturally occurring (e.g, a variant or a random sequence). In one aspect the sequence is contained in a loop region of the tetranectin CRD, and the CRD is fused to a trimerizing domain at the N-terminus or C-terminus of the domain either directly or through the appropriate linker. Also, the fusion protein of the invention may include a second CRD domain, fused at the other of the N-terminus and C-terminus. In a variation of this aspect, the fusion protein includes a polypeptide that binds to a first receptor at one of the termini of the trimerizing domain and includes a CRD the other of the termini. In one aspect, the identification of random or non-natural polypeptides that bind receptors can be accomplished by the method set forth in U.S. Patent Publication No. 2004/0132094 A1.

A vast variety of therapeutic peptides, including the both ligands and receptors, are known in the art to be useful for treating a variety of diseases. Various examples of known targets and indications for therapeutic polypeptides are shown in the following table.

| Peptide Target | Indication | Name/Company |
|---|---|---|
| | Oncology | |
| GNRH receptor | Palliative prostate | Leuprorelin/Takeda |
| | cancer treatment | Histrelin/Valera |
| | | Goserelin/AstraZeneca |
| CXCR4 antagonist | Stem cell | Mozobil/AnorMED Inc |
| | mobilizer, NHL, | (now Genzyme) |
| | MM | |
| | Hepatocellular | CTCE-9908/ |
| | Carcinoma | Chemokine Therapeutics |
| Integrin alphaV-beta 3 | Head and Neck, | Cilengitide/Merck |
| antagonist | glioblastoma | |
| Angiopoietin receptor | Breast, ovarian, | AMG-386 |
| kinase antagonist | renal cell | peptibody/Amgen,Takeda |
| | carcinoma | |
| IGF1-R antagonist | Hepatocellular | Allostera Pharmaceuticals |
| | Carcinoma | |
| Gastrin-releasing | Inflammation, | various |
| peptide receptor | Cancer | |
| bFGF | Anti-angiogenesis | Academic |
| | various cancers | |
| Gelatinase inhibitor | Cancer | CTT Technologies |
| GCSFR agonist | Neutropenia | Gematide/Affymax |
| Keratinocyte GFR | Mucositis | Keratide/Affymax |
| VEGF-R2/c-met | Cancer | Dipeptide from Dyax |
| receptor | | |
| | Autoimmune | |
| TPO | ITP | Nplate peptibody/Amgen |
| GLP-2 analog | Crohn's | Teduglutide/NPS Allelix |
| | Enterocolitis | |
| Treg | MS | Copaxone/Teva |
| GPCR agonists | various | Compugen |
| | Diabetes | |
| GLP-1 analogues/R | | GLP-1 (7- |
| agonists | | 37)/BiorexisPfizer |
| | | Exenatide/Amylin |
| | | Liraglutide/Novo Nordisk |
| | | ZP10/Zealand |
| | | Pharma/Sanofi-Avanetis |
| | | Pramlinitide/Amylin |
| Proislet peptide | | CureDM |
| Glucagon antagonist | | Glucagon |
| | Obesity | PYY/multiple companies |
| Oxyntomodulin | | TKS-1225/Thiakis/Wyeth |
| EPO | Anemic chronic | Hematide/Affimax |
| | kidney disease | |
| Calcitonin | Osteoporosis | Capsitonin/Bone Medical |
| PH receptor | | Teriparatide/Lilly |
| | Cardiovascular | |
| BNP | Congestive heat | Nesiritide/Scios |
| | failure | |
| GIIb/IIIa antagonist | Myocardial | Eptifibatide/COR |
| | infarction | Therapeutics/Schering |
| | | Plough |
| Thrombin inhibitor | Thrombosis, | Bivalirudin/TMC/Scherrer |
| | Ischemic heart | |
| | disease | |
| Bradykinin B2 | Hereditary | Icatibant/Hoechst |
| antagonist | angioedema | |
| GAP junction | Heart Arrhythmia | Rotigaptide/Zealand/Wyeth |
| modulator | | |
| FPLRG1 agonist | Reperfusion injury | Compugen |
| BNP/ANP | Congestive heart | Bispecific/Academic |
| | failure | |
| | Acromegaly | |
| Somatostatin receptor | Acromegaly and | Octreotide/Valera |
| agonist | neuroendocrine | Pharmaceuticals |
| | cancer | Lanreotide/Ibsen |
| | Enuresis | |
| Vasopressin V1 | | Desmopressin/Orphan |
| agonist | | Therapeutics |
| | | Lypressin |
| | | Terlipressin/Orphan |
| | | Therapeutics |
| | Labor | |
| Oxytocin antagonist | Halts Premature | Retosiban/GSK |
| | Labor | Atosiban/Ferring |
| | Antiviral | |
| HIV fusion protein | HIV | Enfuviritide/Roche |
| blocker | | |
| Immunostimulatory | HepC, | Thymalfasin/RegeneRx |
| | Hep C | SCV-07/SciClone |
| CXCR4 antagonist | HIV | AnorMED Inc (now |
| | | Genzyme) |
| CCR5 antagonist | HIV | |
| CXCR4/CCR5 | HIV | Genzyme |
| bispecific | | |
| | Antibacterial | |
| | Staph. aureus | Daptamycin |
| | | Bacitracin |
| | ophthalmic | Gramidicin/Bausch&Lomb |
| | | Colistin |
| | | Pexiganan |
| | | Omiganan |
| | Staph. aureus | Xoma-629 |
| Glycophorin | Malaria | Academic |
| antagonist | | |
| | CNS | |
| Norepinephrine | Severe chronic | Conotoxin/Xenome |
| transporter antagonist | pain | |
| | Antidepressant | Nemifitide |
| Formyl peptide | COPD | Various academics, Bayer |
| receptor-like 1 | | 2003 patent |
| agonists/antagonists | | |
| IL4/IL13 antagonist | Asthma | Synairgen |
| Prokineticin receptor-1 | | Academic |
| and-2 | | |

The truncated tetranectin trimerizing polypeptides of the invention can be used to create trimerized soluble receptors including for example TNF receptor superfamily members, Ig superfamily members, cytokine receptor superfamily members, chemokine receptor superfamily members, integrin family members, growth factor receptor family, hormone receptors, opioid receptors, other neuropeptide receptors, ion channels, among others, including CD1a-e, CD2 (LFA-2), CD2R, CD3γ, CD3δ, CD3ε, CD4-7, CD8a, CD8b, CD9, CD10 CD11a, CD11b, CD11c, CDwl2, CD13, CD14, CD15, CD15s, CD15u, CD16a (FcγRIIIA), CD16b (FcγRIIIB), CDw17, CD18 (Integrin β2), CD19-28, CD29 (Integrin β1), CD30, CD31 (PE-CAM-1), CD32 (FcγRII), CD33 (Siglec-3), CD34-41, CD42a-d, CD43, CD44, CD44R, CD45, CD45RA, CD45RB, CD45RO, DC47, CD47R, CD48, CD49a-f (VLA-1-6), CD50 (ICAM-3), CD51, CD52, CD53, CD54 (ICAM-1), CD55, CD56 (N-CAM), CD57, CD58 (LFA-3), CD59, CD60a-c, CD61, CD62E, CD62L, CD62P, CD63, CD64, CD65, CD65s, CD66a-f, CD68, CD69, CD70, CD71, CD72, CD73, CD74, CD75, CD75s, CD77, CD79a, CD79b, CD80, CD81, CD82, DC83, CDw84, CD85, CD86-CD91, CDw92, CD93, CD94-CD99, CD99R, CD100-CD106, CD107a, CD107b, CD108-CD112, CDw113, CD114 (G-CSFR), CD115 (M-CSFR), CD116, CD117, CD118. CDw119, CD120a, CD120b, CD121a (IL-1R type I), CDw121b (IL-1R, type II), CD122 (IL-2Rβ), CDw123 (IL-3R), CD124 (IL-4R), CDw125 (IL-5R), CD126 (IL-6R), CD127 (IL-7R), CDw128, CDw128b (IL-8Rβ, CD129 (IL-9R), CD130 (IL-6Rβ), CDw131, CD132, CD133, CD134 (Ox-40), CD135-CD139, CD140a (PDGFRα), CD140b (PDGFRβ), CD141-CD144, CDw145, CD146, CD147, CD148, CD15, CD151, CD152 (CTLA-4), CD153 (CD30L), CD154 (CD40L), CD155, CD156a-c, CD157, CD158a, CD158b, CD159a, CD159c, CD160, CD161, CD162, CD162R, CD163, CD164, CD165, CD166, CD167a, CD168, CD169, CD170, CD171, CD172a, CD172b, CD172g, CD173, CD174, CD175, CD175s, CD176, CD178 (FasL), CD179a, CD179b, CD180, CD181 (CXCR1), CD182 (CXCR2), CD183 (CXCR3), CD184 (CXCR4), CD185 (CXCR5), CDw186 (CXCR6), CD191 (CCR-1), CD192 (CCR2), CD193 (CCR3), CD194 (CCR4), CD195 (CCR5). CD196 (CCR6), CD197 (CCR7), CDw198 (CCR8), CDwl99 (CCR9), CD200 (Ox-2), CD201, CD202b, CD203c, CD204 (macrophage scavenger R), CD207 (Langerin), CD208 (DC-LAMP), CD209 (DC-SIGN), CDw210 (IL-10R), CD212 (IL-12-R β1), CD213a1 (IL-13-R α1), CD213a2 (IL-13-R α2), CDw217 (IL-17-R), CDw218a (IL-18Rα), CDw218b (IL-18Rβ), CD220 (Insulin-R), CD221 (IGF-1R), CD222 (IGF-II R), CD223-234, CD235a (glycophorin A), CD235ab (glycophorin A/B), CD235b (glycophorin B), CD236 (glycophorin C/D), CD236R (glycophorin C), CD238, CD239, CD240CE, CD240D, CD241-CD249, CD252 (Ox40L), CD254 (RANKL), CD256 (APRIL), CD257 (BAFF), CD258 (LIGHT), CD261 (TRAIL-R1), CD262 (TRAIL-R2), CD263 (DcR1), CD264 (DcR2), CD256 (RANK), CD266 (TWEAK-R), CD267 (TACI), CD268 (BAFFR), CD269 (BCMA), CD271 (NGFR), CD272 (BTLA), CD273 (PD-L2), CD274 (PD-L1), CD275 (B7-H2), CD276 (B7-H3), CD277, CD278 (ICOS), CD279 (PD1), CD280, CD281 (TLR1), CD282 (TLR2), CD283 (TLR3), CD284 (TLR4), CD289 (TLR9), CD292, CDw293, CD294, CD295 (LeptinR), CD296, CD297, CD298 (Na+/K+-ATPase β3 subunit), CD299 (L-SIGN), CD300a, CD300c, CD300e, CD301-CD307, CD309 (VEGF-R2), CD312, CD314-322, CD324, CDw325, CD326, CDw327, CDw328, CDw329, CD331-CD337, CDw338, CD339, B7-H4, Xedar, CCR10, CCR11, CX3CR1, chemokine-like receptor-1 (ChemR23), complement receptors, DARC, IL-11R, IL-12R, IL-13R, IL-15R, IL-20R, IL-21R, IL-22R, IL-23R, IL-27R, IL-28R, IL-31R, XCR1, CX3CR1, chemokine-binding protein 2 (D6), interferon receptors, leukocyte associated Ig-like receptor family, leukocyte immunoglobulin-like receptor family including LILRC1 and LILRC2, leukotriene receptors, LAMP, nectin-like proteins 1-4, IgSF8, immunoglobulin-like transcript family LT1-6, EDAR, stromal derived factor (SDF), thymic stromal lymphopoietin receptor, erythropoietin receptor, thrombopoietin-receptor, epidermal growth factor receptor, fibroblast growth factor receptors FGF1-4, hepatocyte growth factor receptor (HGF-R), epaCAM, insulin-like growth factor receptors IGF1-R and IGF2-R, fibronectin, fibronectin leucine-rich transmembrane proteins FLRT1-3, Her2, 3 and 4, CRELD1 and 2, 8D6A, lipoprotein receptor (LDL-R), C-type lectin-like family members such as CLEC-1, CLEC-2, CLEC4D, 4F and Dectin 1 and 2, layilin, growth hormone receptor, prolactin-releasing hormone receptor (PRRP), corticotropin-releasing hormone receptors (CRHR), follicle stimulating hormone receptor (FSHR), gonadotropin-releasing hormone receptor (GNRHR), thyrotropin-releasing hormone receptor (TRHR), somatostatin receptors SSTR1-SSTR5, vasopressin receptors 1A, 1B, 2, Oxytocin receptor, luteinizing hormone/choriogonadotropin receptor (LHCGR), thyrotropin receptor, atrial natriuretic factor receptor NPR1-3, acetylcholine receptors (AChR), calcitonin receptor (CT), Cholecystokinin receptors CCKAR and CCKBR, vasoactive intestinal peptide receptors VPAC1 and 2, delta opioid receptor, κ-Opioid receptor, μ opioid receptors, sigma receptors σ1 and σ, cannabinoid receptors R1 and 2, angiotensin receptors AT1-4, bradykinin receptors V1 and 2, tachykinin receptor 1 (TACR1), calcitonin receptor-like receptor (CRLR), galanin receptors R1-3, GPCR neuropeptide receptors neuropeptide B/W R1 and 2, neuropeptide FF receptors R1 and R2, neuropeptide S receptor R1, neuropeptide Y receptors Y1-5, neurotensin receptors, Type I and II activin receptors, activin receptor-like kinases (Alk-1 and Alk-7), betaglycan, BMP and Activin membrane bound inhibitor (BAMBI), cripto, Trk receptors TrkA, TrkB, TrkC, AXL receptor family, LTK receptor family, TIE-1, TIE-2, Ryk, Neuropilin 1, Eph receptors EPHA1, EPHA2, EPHA3, EPHA4, EPHA5, EPHA6, EPHA7, EPHA8, EPHA9, EPHA10, EPHB1, EPHB2, EPHB3, EPHB4, EPHB5, EPHB6, melanocortin receptors MC-3 and MC-4, AMICA, CXADR, corticotrophin-releasing hormone-binding protein, Claa-I restricted T cell-associated molecule, MHCI, MHCII, ampoterin-induced gene and ORF (AMIGOs), APJ, asialoglycoprotein receptors 1 and 2 (ASGPR), brain-specific angiogenesis inhibitor 3 (BAI-3), basal cell adhesion molecule/Lutheran blood group glycoprotein (BCAM/Lu), cadherins, CDCP1, cystic fibrosis transmembrane conductance regulator MRP-7, chondrolectin, lung surfactin, claudins, ANTHXR2, collagens, complement receptors, contactins 1-6, cubulin, endoglycan, EpCAM (epithelial cellular adhesion molecule), Endothelial Protein C receptor (EPCR), Eph receptors, glucagon-like peptide receptors GLP-1R and 2R, glutamate receptors, glucose transporters, glycine receptor, glypicans, G-protein coupled bile acid receptor, G-protein coupled receptor 15, KLOTHO family members, leptin receptor, LIMPII, LINGO, NOGO, lymphatic bessel endothelial hyaluronan receptor 1 (LYVE-1), myeloid inhibitory C-type lectin-like receptor CLEC12A, neogenin, nephrin, NETO-1, NETO-2, NMDA receptor, opioid-binding cell adhesion molecule, osteoclast inhibitory lectin-related protein, oncostatin receptor, osteoclast associated receptor, osteoactivin, thrombin receptors, podoplanin, porimin, potassium channels, Pref-1, stem cell factor receptor, semaphorins, SPARC, scavenger receptor A1, stabilins, syndecans, T cell receptors, TCAM-1, T cell cytokine receptor TCCR, thrombospondins, TIM1-6, toll-like receptors, triggering receptors expressed on myeloid cells (TREM) and TREM-like proteins, TROP-2 or any mimetic or analog thereof.

Furthermore, the truncated tetranectin trimerizing polypeptides of the invention can be used to trimerize ligands of any of the above receptors including for example TNF superfamily members, cytokine superfamily members, growth factors, chemokine superfamily members, pro-angiogenic factors, pro-apoptotic factors, integrins, hormones and other soluble factors, among others, including RANK-L, Lymphotoxin (LT)-α, LT-β, LT-α1β2, zLIGHT, BTLA. TL1A, FasL, TWEAK, CD30L, 4-1BB-L (CD137L), CD27L, Ox40L (CD134L), GITRL, CD40L (CD154), APRIL (CD256), BAFF, EDA1, IL-1α, IL-1β, IL-1RA, IL-2, IL-3, IL-4, IL-5, IL-6, IL-7, IL-8, IL-9, IL-10, IL-11, IL-12, IL-13, IL-14, IL-15, IL-16, IL-17A, IL-17F, IL-17A/F, IL-18, IL-1 g, IL-20, IL-21, IL-22, IL-23, IL-24, IL-25, IL-26, IL-27, IL-28, IL-29, IL-30, IL-31, IL-32, IL-33, IL-34, IL-35, IFN-gamma, IFN-alpha, IFN-beta, TNF-α, TNF-β, G-CMF, GM-CSF, TGF-β1, 2 and 3, TGF-α, cardiotrophin-1, leukemia inhibitory factor (LIF), betacellulin, amphiregulin, thymic stromal lymphopoietin (TSLP), flt-3, CXCL1-16, CCL1-3, CCL3L1, CCL4-CCL8, CCL9/10, CCL11-28, XCL1, XCL2, CX3CL1, HMG-B1, heat shock proteins, chemerin, defensins, macrophage migration inhibitory factor (MIF), oncostatin M, limitin, vascular endothelial growth factors VEGF A-D and PIGF, lens epithelium derived growth factor, erythropoietin, thrombopoietin, platelet derived growth factor, epidermal growth factor, fibroblast growth factors FGF1-14 and 16-23, hepatoma-derived growth factor, hepassocin, hepatocyte growth factor, platelet-derived endothelial growth factor (PD-ECGF), insulin-like growth factors IGF1 and IGF2, IGF binding proteins (IGFBP 1-6), GASPS (growth and differentiation-factor-associated serum proteins), connective tissue growth factor, epigen, epiregulin, developmental arteries and neural crest epidermal growth factor (DANCE), glial maturation factor-β, insulin, growth hormone, angiogenin, angiopoietin 1-4, angiopoietin-like proteins 1-4, integrins αVβ3, αVβ5 and α5β1, erythropoietin, thrombopoietin, prolactin releasing hormone, corticotropin-releasing hormone (CRH), gonadotropin releasing hormone, thyrotropin releasing hormone, somatostatin, vasopressin, oxytocin, demoxytocin, carbetocin, luteinizing hormone (LH) and chorionic gonadotropins, thyroid-stimulating hormone, ANP, BNP, CNP, calcitonin, CCK a, CCK B, vasoactive intestinal peptides 1 and 2, enkephalin, dynorphin, beta-endorphin, morphine, 4-PPBP, [1] SA 4503, Ditolylguanidine, siramesine angiotensin, kallidin, bradykinin, tachykinins, substance P, calcitonin, galanin, neurotensin, neuropeptides Y1-5, neuropeptide S, neuropeptide FF, neuropeptide B/W, brain-derived neurotrophic factors BDNF, NT-3, NT-4/5, activin A, AB, B and C, inhibin, Müllerian inhibiting hormone (MIH), bone morphogenetic proteins BMP2, BMP3, BMP4, BMP5, BMP6, BMP7, BMP8a, BMP8b, BMP10, BMP15, growth differentiating factors GDF1, GDF2, GDF3, GDF5, GDF6, GDF7, Myostatin/GDF8, GDF9, GDF10, GDF11, GDF15, nerve growth factor (NGF), brain-derived neurotrophic factor (BDNF), neurotrophin-3 (NT-3), and neurotrophin-4 (NT-4), artemin, persephin, neurturin, GDNF, agrin, ephrin ligands EFNA1, EFNA2, EFNA3, EFNA4, EFNA5 EFNB1, EFNB2, EFNB3, adiponectin, α2-macroglobulin, agrecan, agouti-related protein (AgRP), α-melanocyte stimulating hormone, albumin, ameloblastin, plasminogen, angiostatin, apolipoproteins A1, AII, B, B100, E, amyloid, autophagin, TGF-beta induced protein Ig H3), biglycan, leukocyte cell-derived chemotaxin LECT2, C-reactive protein, complement components, chordin, chordin-like proteins, collectins, clusterin-like protein 1, cortisol, van willebrandt factor, cytostatins, endostatin, endoreppellin, ephrin ligands, fetuins, ficolins, glucagon, granulysin, gremlin, HGF activator inhibitors HAI-1 and 2, kallilcreins, laminins, leptins, lipocalins, mannan binding lectins (MBL), meteorin, MFG-E8, macrophage galactose N-acetyl-galactosamine-specific lectin (MGL), midkine, myocilin, nestin, osteoblast-specific factor 2, osteopontin, osteocrin, osteoadherin, pentraxin, persephin, placenta growth factor, relaxins, resistin and resistin-like molecules, stem cell factor, stanniocalcins, VE-statin, substance P, tenascins, vitronectin, tissue factor, tissue factor pathway inhibitors, as well as any other of the >7000 proteins identified in the human secretome as listed in the secreted protein database (Chen Y, Zhang Y, Yin Y, Gao G, Li S, Jiang Y, Gu X, Luo J (2005) SPD—a web-based secreted protein database. Nucleic Acids Res 33 Database Issue:D169-173), or any mimetic or analog thereof.

Furthermore, the truncated tetranectin trimerizing polypeptides of the invention can be used to trimerize enzymes such as for example angiotensin converting enzymes (ACE), matrix metalloproteases, ADAM metalloproteases with thrombospondin type I motif (ADAMTS1, 4, 5, 13), aminopeptidases, beta-site APP-cleaving enzymes (BACE-1 and -2), chymase, kallilkreins, reelin, serpins, or any mimetic or analog thereof.

Furthermore, the truncated tetranectin trimerizing polypeptides of the invention can be used to trimerize chemotherapeutic agents, and toxins such as small molecule toxins or enzymatically active toxins of bacterial, fungal, plant or animal origin, or fragments thereof to increase potency of targeted compounds for therapeutic purposes, such as for example calicheamicin, pseudomonas exotoxin, diphteria toxin, ricin, saporin, apoptosis-inducing peptides or any analog thereof.

The truncated tetranectin trimerizing polypeptides of the invention can also be used to fuse viral fusion proteins thereby preventing fusion of viruses with cells, such as e.g. the HR2 domain of gp41 (HIV), HA2 domain of influenza virus hemagglutinin, flavivirus envelope protein E, alphavilus fusion protein E1, dengue virus fusion domains, HCV fusion domains, vesicular stomatitis virus fusion domains, herpesvirus fusion protein gB and gD, among others.

The truncated tetranectin trimerizing polypeptides of the invention can also be used to fuse antigens for cancer vaccines such as for example the colorectal cancer antigen A33, α-fetoprotein, mucin 1 (MUC1), CDCP1, carcinoembryonic antigen cell adhesion molecules, Her-2, 3 and 4, mesothelin, CDCP1, NETO-1, NETO-2, syndecans, LewisY, CA-125, melanoma associated antigen (MAGE), tyrosinase, epithelial tumor antigen (ETA), among others, as well as for fusing viral envelope antigens or fungal antigens for treatment of infectious diseases.

In one particular example, the invention is directed to a method for treating an interleukin-1 mediated disease. As used herein, a disease or medical condition is considered to be an “interleukin-1 mediated disease” or “a disease mediated by interleukin-1” if the spontaneous or experimental disease or medical condition is associated with elevated levels of IL-1 in bodily fluids or tissue or if cells or tissues taken from the body produce elevated levels of IL-1 in culture. In many cases, such interleukin-1 mediated diseases are also recognized by the following additional two conditions: (1) pathological findings associated with the disease or medical condition can be mimicked experimentally in animals by the administration of IL-1; and (2) the pathology induced in experimental animal models of the disease or medical condition can be inhibited or abolished by treatment with agents which inhibit the action of IL-1. In most IL-1 mediated diseases at least two of the three conditions are met, and in many IL-1 mediated diseases all three conditions are met. A non-exclusive list of acute and chronic IL-1-mediated inflammatory diseases includes but is not limited to the following: gout, acute pancreatitis; ALS; Alzheimer's disease; cachexia/anorexia; asthma; atherosclerosis; chronic fatigue syndrome, fever; diabetes (e.g., insulin diabetes); glomerulonephritis; graft versus host rejection; hemohorragic shock; hyperalgesia, inflammatory bowel disease; inflammatory conditions of a joint, including osteoarthritis, psoriatic arthritis, juvenile arthritis, and rheumatoid arthritis; ischemic injury, including cerebral ischemia (e.g., brain injury as a result of trauma, epilepsy, hemorrhage or stroke, each of which may lead to neurodegeneration); lung diseases (e.g., ARDS); multiple myeloma; multiple sclerosis; myelogenous (e.g., AML and CML) and other leukemias; myopathies (e.g., muscle protein metabolism, esp. in sepsis); osteoporosis; Parkinson's disease; pain; pre-term labor; psoriasis; reperfusion injury; septic shock; side effects from radiation therapy, temporal mandibular joint disease, tumor metastasis; or an inflammatory condition resulting from strain, sprain, cartilage damage, trauma, orthopedic surgery, infection or other disease processes, and Cryopyrin-associated periodic syndromes, including Muckle Wells syndrome, familial cold autoinflammatory syndrome and neonatal-onset multisystem inflammatory disease.

The IL-1 Ra polypeptide of the invention may either be linked to the N- or the C-terminal amino acid residue of the trimerization domain. A flexible molecular linker optionally may be interposed between, and covalently join, the polypeptide representing the IL-1 Ra and the trimerization domain. Preferably, the linker is a polypeptide sequence of about 1 to 20, 2 to 10, or 3 to 7 amino acid residues. In further embodiments, the linker is non-immunogenic, not prone to proteolytic cleavage, and does not comprise amino acid residues which are known to interact with other residues (e.g. cystein residues).

As used herein “IL-1 Ra” refers to a polypeptide having the amino acid sequence shown below:

(SEQ ID NO: 21)

RPSGRKSSKMQAFRIWDVNQKTFYLRNNQLVAGYLQGPNVNLEEKIDVVP
IEPHALFLGIHGGKMCLSCVKSGDETRLQLEAVNITDLSENRKQDKRFAF
IRSDSGPTTSFESAACPGWFLCTAMEADQPVSLTNMPDEGVMVTKFYFQE
DE

Also included in the “IL-1Ra” definition are variants and fragments of SEQ ID NO: 38 that provide for IL-1Ra binding to IL-1R, and preferably IL-1R inhibitory activity. Such fragments may be truncated at the N-terminus or C-terminus of the IL-1Ra, or may lack internal residues, when compared with the full length native IL-1Ra protein. Certain fragments may lack amino acid residues that are not essential for a desired biological activity of the trimeric IL-1 Ra protein according to the invention. For example, Evans, et al. (J. Biol. Chem. 1995, 19:11477-11483) demonstrated by site directed mutagenesis that only Trp16, Gln20, Tyr34, Gln36 and Tyr147 are critical for binding to the IL-1R and that other amino acid positions can be altered while still maintaining a functional molecule. Furthermore, affinity of IL-1Ra to its receptor can be improved by mutating amino acids outside the binding region to increase loop interactions of IL-1Ra with its receptor as shown by Dahlen, et al, (J. Immunotoxicology 5:189-199 (2008)). This is can be accomplished through mutations of amino acids outside the IL-1Ra receptor binding region, and particularly, for example: D47N, E52R, E90Y, P38Y, H54R, Q129L and M136N. Id. Furthermore, natural IL-1Ra variants exist, any of which may be used. An 18 kDa form of IL-1Ra, created by an alternative transcriptional splice mechanism from an upstream exon is called icIL-1Ra1 and is found inside keratinocytes and other epithelial cells, monocytes, tissue macrophages, fibroblasts, and endothelial cells. IL-1Ra cDNA cloned from human leukocytes contains an additional 63 bp sequence as an insert in the 5′ region of the cDNA. A 15 kDa isoform of IL-1Ra, termed icIL-1Ra3, is found in monocytes, macrophages, neutrophils, and hepatocytes, and may be created both by an alternative transcriptional splice as well as by alternative translational initiation.

IL-1Ra peptides that are useful for fusion proteins of the invention include polypeptides that are at least 65%, at least 70%, at least 75%, at least 80%, at least 85%, at least 90%, or at least 95% identical to SEQ ID NO: 38. In particular embodiments, the fusion proteins include an IL-1Ra peptide sequence that is 85% identical to SEQ ID NO: 38 and has IL-1R binding activity, and preferably IL-1Ra inhibitory activity. In another particular embodiment, the fusion proteins include an IL-1Ra peptide sequence that is 95% identical to SEQ ID NO: 38 and has IL-1R binding activity, and preferably IL-1Ra inhibitory activity. In these embodiment, the polypeptides comprise Trp16, Gln20, Tyr34, Gln36 and Tyr147 according to the numbering of SEQ ID NO: 38 These polypeptides may further include one or more amino acids substitutions D47N, E52R, E90Y, P38Y, H54R, Q129L and M136N (numbering according to SEQ ID NO: 38). Furthermore, variations of the IL-1Ra polypeptides can be accomplished by replacing one or more amino acids with another amino acid having similar structural or chemical properties, for example, conservative amino acid substitutions.

In a further embodiment, the fusion protein according to the invention is selected from an IL-1 receptor antagonist selected from the following:

TripK-IL-1ra

(SEQ ID NO: 22)

EGPTQKPKKIVNAKKDVVNTKMFEELKSRLDTLAQEVALLKEQQALQTVS
LKRPSGRKSSKMQAFRIWDVNQKTFYLRNNQLVAGYLQGPNVNLEEKIDV
VPIEPHALFLGIHGGKMCLSCVKSGDETRLQLEAVNITDLSENRKQDKRF
AFIRSDSGPTTSFESAACPGWFLCTAMEADQPVSLTNMPDEGVMVTKFYF
QEDE;
TripV-IL-1ra

(SEQ ID NO: 23)

EGPTQKPKKIVNAKKDVVNTKMFEELKSRLDTLAQEVALLKEQQALQTVR
PSGRKSSKMQAFRIWDVNQKTFYLRNNQLVAGYLQGPNVNLEEKIDVVPE
PHALFLGIHGGKMCLSCVKSGDETRLQLEAVNITDLSENRKQDKRFAFIR
SDSGPTTSFESAACPGWFLCTAMEADQPVSLTNMPDEGVMVTKFYFQED
E;
TripT-IL-1ra

(SEQ ID NO: 24)

EGPTQKPKKIVNAKKDVVNTKMFEELKSRLDTLAQEVALLKEQQALQTRP
SGRKSSKMQAFRIWDVNQKTFYLRNNQLVAGYLQGPNVNLEEKIDVVPIE
PHALFLGIHGGKMCLSCVKSGDETRLQLEAVNITDLSENRKQDKRFAFIR
SDSGPTTSFESAACPGWFLCTAMEADQPVSLTNMPDEGVMVTKFYFQEDE
TripQ-IL-1ra

(SEQ ID NO: 25)

EGPTQKPKKIVNAKKDVVNTKMFEELKSRLDTLAQEVALLKEQQALQRPS
GRKSSKMQAFRIWDVNQKTFYLRNNQLVAGYLQGPNVNLEEKIDVVPIEP
HALFLGIHGGKMCLSCVKSGDETRLQLEAVNITDLSENRKQDKRFAFIRS
DSGPTTSFESAACPGWFLCTAMEADQPVSLTNMPDEGVMVTKFYFQEDE
I10-TripK-IL1ra

(SEQ ID NO: 26)

IVNAKKDVVNTKMFEELKSRLDTLAQEVALLKEQQALQTVSLKRPSGRKS
SKMQAFRIWDVNQKTFYLRNNQLVAGYLQGPNVNLEEKIDVVPIEPHALF
LGIHGGKMCLSCVKSGDETRLQLEAVNITDLSENRKQDKRFAFIRSDSGP
TTSFESAACPGWFLCTAMEADQPVSLTNMPDEGVMVTKFYFQEDE;
I10-TripV-IL-1ra

(SEQ ID NO: 27)

IVNAKKDVVNTKMFEELKSRLDTLAQEVALLKEQQALQTVRPSGRKSSKM
QAFRIWDVNQKTFYLRNNQLVAGYLQGPNVNLEEKIDVVPIEPHALFLGI
HGGKMCLSCVKSGDETRLQLEAVNITDLSENRKQDKRFAFIRSDSGPTTS
FESAACPGWFLCTAMEADQPVSLTNMPDEGYMVTKFYFQEDE;
I10-TripT-IL-1ra

(SEQ ID NO: 28)

IVNAKKDVVNTKMFEELKSRLDTLAQEVALLKEQQALQTRPSGRKSSKMQ
AFRIWDVNQKTFYLRNNQLVAGYLQGPNVNLEEKIDVVPLEPHALFLGIH
GGKMCLSCVKSGDETRLQLEAVNITDLSENRKQDKRFAFIRSDSGPTTSF
ESAACPGWFLCTAMEADQPVSLTNMPDEGVMVTKFYFQEDE;
I10-TripQ-IL-1ra

(SEQ ID NO: 29)

IVNAKKDVVNTKMFEELKSRLDTLAQEVALLKEQQALQRPSGRKSSKMQA
FRIWDVNQKTFYLRNNQLVAGYLQGPNVNLEEKIDVYPIEPHALFLGIHG
GKMCLSCVKSGDETRLQLEAVNITDLSENRKQDKRFAFIRSDSGPTTSFE
SAACPGWFLCTAMEADQPYSLTNMPDEGVMVTKFYFQEDE;
V17-TripK-IL1ra

(SEQ ID NO: 30)

VVNTKMFEELKSRLDTLAQEVALLKEQQALQTVSLKRPSGRKSSKMQAFR
IWDVNQKTFYLRNNQLVAGYLQGPNVNLEEKIDVVPIEPHALFLGIHGGK
MCLSCVKSGDETRLQLEAVNITDLSENRKQDKRFAFIRSDSGPTTSFESA
ACPGWFLCTAMEADQPVSLTNMPDEGVMVTKFYFQEDE;
V17-TripV-IL-1ra

(SEQ ID NO: 31)

VVNTKMFEELKSRLDTLAQEVALLKEQQALQTVRPSGRKSSKMQAFRIWD
VNQKTFYLRNNQLVAGYLQGPNVNLEEKIDVVPIEPHALFLGIHGGKMCL
SCVKSGDETRLQLEAVNITDLSENRKQDKRFAFIRSDSGPTTSFESAACP
GWFLCTAMEADQPVSLTNMPDEGVMVTKFYFQEDE;
V17-TripT-IL-1ra

(SEQ ID NO: 32)

VVNTKMFEELKSRLDTLAQEVALLKEQQALQTRPSGRKSSKMQAFRIWDV
NQKTFYLRNNQLVAGYLQGPNVNLEEKIDVVPIEPHALFLGIHGGKMCLS
CVKSGDETRLQLEAVNITDLSENRKQDKRFAFIRSDSGPTTSFESAACPG
WFLCTAMEADQPYSLTNMPDEGVMVTKFYFQEDE;
V17-TripQ-IL-1ra

(SEQ ID NO: 33)

VYNTKMFEELKSRLDTLAQEVALLKEQQALQRPSGRKSSKMQAFRIWDVN
QKTFYLRNNQLVAGYLQGPNVNLEEKIDVVPIEPHALFLGIHGGKMCLSC
VKSGDETRLQLEAVNITDLSENRKQDKRFAFIRSDSGPTTSFESAACPGW
FLCTAMEADQPVSLTNMPDEGVMVTKFYFQEDE;

wherein the underlined part denotes the trimerization unit, and the bold part denotes the IL-1Ra part.

### Production of Fusion Proteins

The trimeric IL-1 Ra protein of the invention may be chemically synthesized or expressed in any suitable standard protein expression system. Preferably, the protein expression systems are systems from which the desired protein may readily be isolated and refolded in vitro. Prokaryotic expression systems are preferred since high yields of protein can be obtained and efficient purification and refolding strategies are available. Eukaryotic expression systems may also be used. Thus, it is well within the abilities and discretion of the skilled artisan to choose an appropriate expression system. Similarly, once the primary amino acid sequence for the fusion proteins of the present invention is chosen, one of ordinary skill in the art can easily design appropriate recombinant DNA constructs which will encode the desired proteins, taking into consideration such factors as codon biases in the chosen host, the need for secretion signal sequences in the host, the introduction of proteinase cleavage sites within the signal sequence, and the like. These recombinant DNA constructs may be inserted in-frame into any of a number of expression vectors appropriate to the chosen host. Preferably, the expression vector will include a strong promoter to drive expression of the recombinant constructs.

The fusion protein of the invention can be expressed in any suitable standard protein expression system by culturing a host transformed with a vector encoding the fusion protein under such conditions that the fusion protein is expressed. Preferably, the expression system is a system from which the desired protein may readily be isolated and refolded in vitro. As a general matter, prokaryotic expression systems are preferred since high yields of protein can be obtained and efficient purification and refolding strategies are available. Thus, selection of appropriate expression systems (including vectors and cell types) is within the knowledge of one skilled in the art. Similarly, once the primary amino acid sequence for the fusion protein of the present invention is chosen, one of ordinary skill in the art can easily design appropriate recombinant DNA constructs which will encode the desired amino acid sequence, taking into consideration such factors as codon biases in the chosen host, the need for secretion signal sequences in the host, the introduction of proteinase cleavage sites within the signal sequence, and the like.

In one embodiment the isolated polynucleotide encodes a fusion protein of the invention. In other embodiments, an IL-1Ra polypeptide and the trimerizing domain are encoded by non-contiguous polynucleotide sequences. Accordingly, in some embodiments an IL-1Ra polypeptide and the trimerizing domain are expressed, isolated, and purified as separate polypeptides and fused together to form the fusion protein of the invention.

These recombinant DNA constructs may be inserted in-frame into any of a number of expression vectors appropriate to the chosen host. In certain embodiments, the expression vector comprises a strong promoter that controls expression of the recombinant fusion protein constructs. When recombinant expression strategies are used to generate the fusion protein of the invention, the resulting fusion protein can be isolated and purified using suitable standard procedures well known in the art, and optionally subjected to further processing such as e.g. lyophilization.

Standard techniques may be used for recombinant DNA molecule, protein, and fusion protein production, as well as for tissue culture and cell transformation. See, e.g., Sambrook, et al., Molecular Cloning: A Laboratory Manual. Cold Spring Harbor Laboratory Press, Cold Spring Harbor, N.Y. (1989) or Current Protocols in Molecular Biology (Ausubel et al., eds., Green Publishers Inc. and Wiley and Sons 1994). Purification techniques are typically performed according to the manufacturer's specifications or as commonly accomplished in the art using conventional procedures such as those set forth in Sambrook et al., or as described herein. Unless specific definitions are provided, the nomenclature utilized in connection with the laboratory procedures, and techniques relating to molecular biology, biochemistry, analytical chemistry, and pharmaceutical/formulation chemistry described herein are those well imown and commonly used in the art. Standard techniques can be used for biochemical syntheses, biochemical analyses, pharmaceutical preparation, formulation, and delivery, and treatment of patients.

It will be appreciated that a flexible molecular linker optionally may be interposed between, and covalently join, the therapeutic polypeptide and the trimerizing domain. In certain embodiments, the linker is a polypeptide sequence of about 1 to 20 amino acid residues. The linker may be less than 10 amino acids, most preferably, five, four, three, two, or one amino acid. It may be in certain cases that nine, eight, seven, or six amino acids are suitable. In useful embodiments the linker is essentially non-immunogenic, not prone to proteolytic cleavage and does not comprise amino acid residues which are known to interact with other residues (e.g. cysteine residues). In certain embodiments the linker contains proteosomal cleavage sites for intracellular release of toxic molecules.

The description below also relates to methods of producing fusion proteins and trimeric complexes that are covalently attached (hereinafter “conjugated”) to one or more chemical groups. Chemical groups suitable for use in such conjugates are preferably not significantly toxic or immunogenic. The chemical group is optionally selected to produce a conjugate that can be stored and used under conditions suitable for storage. A variety of exemplary chemical groups that can be conjugated to polypeptides are known in the art and include for example carbohydrates, such as those carbohydrates that occur naturally on glycoproteins, polyglutamate, and non-proteinaceous polymers, such as polyols (see, e.g., U.S. Pat. No. 6,245,901).

A polyol, for example, can be conjugated to fusion proteins of the invention at one or more amino acid residues, including lysine residues, as is disclosed in WO 93/00109, supra. The polyol employed can be any water-soluble poly(alkylene oxide) polymer and can have a linear or branched chain. Suitable polyols include those substituted at one or more hydroxyl positions with a chemical group, such as an alkyl group having between one and four carbons. Typically, the polyol is a poly(alkylene glycol), such as poly(ethylene glycol) (PEG), and thus, for ease of description, the remainder of the discussion relates to an exemplary embodiment wherein the polyol employed is PEG and the process of conjugating the polyol to a polypeptide is termed “pegylation.” However, those skilled in the art recognize that other polyols, such as, for example, poly(propylene glycol) and polyethylene-polypropylene glycol copolymers, can be employed using the techniques for conjugation described herein for PEG.

The average molecular weight of the PEG employed in the pegylation of IL-1Ra can vary, and typically may range from about 500 to about 30,000 daltons (D). Preferably, the average molecular weight of the PEG is from about 1,000 to about 25,000 D, and more preferably from about 1,000 to about 5,000 D. In one embodiment, pegylation is carried out with PEG having an average molecular weight of about 1,000 D. Optionally, the PEG homopolymer is unsubstituted, but it may also be substituted at one end with an alkyl group. Preferably, the alkyl group is a C1-C4 alkyl group, and most preferably a methyl group. PEG preparations are commercially available, and typically, those PEG preparations suitable for use in the present invention are non-homogeneous preparations sold according to average molecular weight. For example, commercially available PEG(5000) preparations typically contain molecules that vary slightly in molecular weight, usually ±500 D. The fusion protein of the invention can be further modified using techniques known in the art, such as, conjugated to a small molecule compounds (e.g., a chemotherapeutic); conjugated to a signal molecule (e.g., a fluorophore); conjugated to a molecule of a specific binding pair (e.g., biotin/streptavidin, antibody/antigen); or stabilized by glycosylation, PEGylation, or further fusions to a stabilizing domain (e.g., Fc domains).

A variety of methods for pegylating proteins are known in the art. Specific methods of producing proteins conjugated to PEG include the methods described in U.S. Pat. Nos. 4,179,337, 4,935,465 and 5,849,535. Typically the protein is covalently bonded via one or more of the amino acid residues of the protein to a terminal reactive group on the polymer, depending mainly on the reaction conditions, the molecular weight of the polymer, etc. The polymer with the reactive group(s) is designated herein as activated polymer. The reactive group selectively reacts with free amino or other reactive groups on the protein. The PEG polymer can be coupled to the amino or other reactive group on the protein in either a random or a site specific manner. It will be understood, however, that the type and amount of the reactive group chosen, as well as the type of polymer employed, to obtain optimum results, will depend on the particular protein or protein variant employed to avoid having the reactive group react with too many particularly active groups on the protein. As this may not be possible to avoid completely, it is recommended that generally from about 0.1 to 1000 moles, preferably 2 to 200 moles, of activated polymer per mole of protein, depending on protein concentration, is employed. The final amount of activated polymer per mole of protein is a balance to maintain optimum activity, while at the same time optimizing, if possible, the circulatory half-life of the protein.

The term “polyol” when used herein refers broadly to polyhydric alcohol compounds. Polyols can be any water-soluble poly(alkylene oxide) polymer for example, and can have a linear or branched chain. Preferred polyols include those substituted at one or more hydroxyl positions with a chemical group, such as an alkyl group having between one and four carbons. Typically, the polyol is a poly(alkylene glycol), preferably poly(ethylene glycol) (PEG). However, those skilled in the art recognize that other polyols, such as, for example, poly(propylene glycol) and polyethylene-polypropylene glycol copolymers, can be employed using the techniques for conjugation described herein for PEG. The polyols of the invention include those well known in the art and those publicly available, such as from commercially available sources.

Furthermore, other half-life extending molecules can be attached to the N- or C-terminus of the trimerization domain including serum albumin-binding peptides, FcRn-binding peptides or IgG-binding peptides.

In one embodiment, the trimeric protein of the invention is expressed in a prokaryotic host cell such as E. coli and is additionally linked to a third polypeptide, i.e. a third fusion partner. Thus, it may be that by adding such third fusion partner to the trimeric protein of the invention, high yields of the trimeric protein may be obtained. The third fusion partner may be any suitable peptide, oligopeptide, polypeptide or protein, including a di-peptide, a tri-peptide, tetra-peptide, penta-peptide or hexa-peptide. The fusion partner may in certain instances be a single amino acid. It may be selected such that it renders the fusion protein more resistant to proteolytic degradation, facilitates enhanced expression and secretion of the fusion protein, improves solubility, and/or allows for subsequent affinity purification of the fusion protein.

In one embodiment, the junction region between the fusion protein of the invention (i.e. the IL-1Ra portion and the trimerization domain) and the third fusion partner such as ubiquitin, comprises a Granzyme B protease cleavage site such as human Granzyme B (E.C. 3.4.21.79) as described in US 2006/0199251.

The third fusion partner may in further embodiments be coupled to an affinity-tag. Such an affinity-tag may be an affinity domain which allows for the purification of the fusion protein on an affinity resin. The affinity-tag may be a polyhistidine-tag such as a hexahis-tag, polyarginine-tag, FLAG-tag, Strep-tag, c-myc-tag, S-tag, calmodulin-binding peptide, cellulose-binding peptide, chitin-binding domain, glutathione S-transferase-tag, or maltose binding protein.

The method of the invention may be in an isolation step for isolating the trimeric IL-1 Ra protein that is formed by the enzymatic cleavage of the fusion protein that has been immobilized by the use of the above mentioned affinity-tag systems. This isolation step can be performed by any suitable means known in the art for protein isolation, including the use of ion exchange and fractionation by size, the choice of which depends on the character of the fusion protein. In one embodiment, the region between the third fusion partner and the region comprising the trimerization domain and therapeutic polypeptide is contacted with the human serine protease Granzyme B to cleave off the fusion protein at a Granzyme B protease cleavage site which yields the fusion protein of the invention.

The present invention also provides plasmids, vectors, transcription or expression cassettes which comprise at least one nucleic acid as described above. Suitable vectors can be chosen or constructed containing appropriate regulatory sequences, including promoter sequences, terminator sequences, polyadenylation sequences, enhancer sequences, marker genes and other sequences as appropriate. Vectors may be plasmids, viral, phage, or phagemid, as appropriate. (Molecular Cloning: a Laboratory Manual: 2nd edition, Sambrook et al., 1989, Cold Spring Harbor Laboratory Press).

The present invention also provides a recombinant host cell which comprises one or more constructs of the invention. Suitable host cells include bacteria, mammalian cells, yeast and baculovirus systems. Mammalian cell lines available for expression of a heterologous polypeptide include Chinese hamster ovary cells, HeLa cells, baby hamster kidney cells, NSO mouse melanoma cells and many others. One bacterial host is E. coli. Also, mammalian expression is desirable for molecules having glycosylation sites in order to provide for glycosylation of the peptide.

Pharmaceutical Compositions

In yet another aspect, the invention relates to a pharmaceutical composition comprising a therapeutically effective amount of the fusion protein of the invention along with a pharmaceutically acceptable carrier or excipient. As used herein, “pharmaceutically acceptable carrier” or “pharmaceutically acceptable excipient” includes any and all solvents, dispersion media, coating, antibacterial and antifungal agents, isotonic and absorption delaying agents, and the like that are physiologically compatible. Examples of pharmaceutically acceptable carriers or excipients include one or more of water, saline, phosphate buffered saline, dextrose, glycerol, ethanol and the like as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the of the antibody or antibody portion also may be included. Optionally, disintegrating agents can be included, such as cross-linked polyvinyl pyrrolidone, agar, alginic acid or a salt thereof, such as sodium alginate and the like. In addition to the excipients, the pharmaceutical composition can include one or more of the following, carrier proteins such as serum albumin, buffers, binding agents, sweeteners and other flavoring agents; coloring agents and polyethylene glycol.

The compositions can be in a variety of forms including, for example, liquid, semi-solid and solid dosage forms, such as liquid solutions (e.g. injectable and infusible solutions), dispersions or suspensions, tablets, pills, powders, liposomes and suppositories. The preferred form will depend on the intended route of administration and therapeutic application. In an embodiment the compositions are in the form of injectable or infusible solutions, such as compositions similar to those used for passive immunization of humans with antibodies. In an embodiment the mode of administration is parenteral (e.g., intravenous, subcutaneous, intraperitoneal, intramuscular). In an embodiment, the fusion protein (or trimeric complex) is administered by intravenous infusion or injection. In another embodiment, the fusion protein or trimeric complex is administered by intramuscular or subcutaneous injection.

Other suitable routes of administration for the pharmaceutical composition include, but are not limited to, oral, rectal, transdermal, vaginal, transmucosal, intraarticular or intestinal administration.

Therapeutic compositions are typically sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e. fusion protein or trimeric complex) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

An article of manufacture such as a kit containing therapeutic agents useful in the treatment of the disorders described herein comprises at least a container and a label. Suitable containers include, for example, bottles, vials, syringes, and test tubes. The containers may be formed from a variety of materials such as glass or plastic. The label on or associated with the container indicates that the formulation is used for treating the condition of choice. The article of manufacture may further comprise a container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use. The article of manufacture may also comprise a container with another active agent as described above.

Typically, an appropriate amount of a pharmaceutically-acceptable salt is used in the formulation to render the formulation isotonic. Examples of pharmaceutically-acceptable carriers include saline, Ringer's solution and dextrose solution. The pH of the formulation is preferably from about 6 to about 9, and more preferably from about 7 to about 7.5. It will be apparent to those persons skilled in the art that certain carriers may be more preferable depending upon, for instance, the route of administration and concentrations of therapeutic agent.

Therapeutic compositions can be prepared by mixing the desired molecules having the appropriate degree of purity with optional pharmaceutically acceptable carriers, excipients, or stabilizers (Remington's Pharmaceutical Sciences, 16th edition, Osol, A. ed. (1980)), in the form of lyophilized formulations, aqueous solutions or aqueous suspensions. Acceptable carriers, excipients, or stabilizers are preferably nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, colloidal silica, magnesium trisilicate, polyvinyl pyrrolidone, and cellulose-based substances. Carriers for topical or gel-based forms include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations.

Formulations to be used for in vivo administration should be sterile. This is readily accomplished by filtration through sterile filtration membranes, prior to or following lyophilization and reconstitution. The formulation may be stored in lyophilized form or in solution if administered systemically. If in lyophilized form, it is typically formulated in combination with other ingredients for reconstitution with an appropriate diluent at the time for use. An example of a liquid formulation is a sterile, clear, colorless unpreserved solution filled in a single-dose vial for subcutaneous injection.

Therapeutic formulations generally are placed into a container having a sterile access port, for example, an intravenous solution bag or vial having a stopper pierceable by a hypodermic injection needle. The formulations are preferably administered as repeated intravenous (i.v.), subcutaneous (s.c.), intramuscular (i.m.) injections or infusions, or as aerosol formulations suitable for intranasal or intrapulmonary delivery (for intrapulmonary delivery see, e.g., EP 257,956).

The molecules disclosed herein can also be administered in the form of sustained-release preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res., 15: 167-277 (1981) and Langer, Chem. Tech., 12: 98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the Lupron Depot (injectable microspheres composed of lactic acid-glycolic acid copolymer and leuprolide acetate), and poly-D-(−)-3-hydroxybutyric acid (EP 133,988).

Methods of Treatment

Another aspect the invention relates to a method of treating diseases that are mediated by receptors or antagonists of the therapeutic proteins of the invention. The method includes treating a subject suffering from such as disease with a therapeutically effective amount of the pharmaceutical compositions of the invention.

Another aspect of the invention is directed to a combination therapy. Formulations comprising therapeutic agents are also provided by the present invention. It is believed that such formulations will be particularly suitable for storage as well as for therapeutic administration. The formulations may be prepared by known techniques. For instance, the formulations may be prepared by buffer exchange on a gel filtration column.

The pharmaceutical compositions can be administered in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Optionally, administration may be performed through mini-pump infusion using various commercially available devices.

For example, effective dosages and schedules for administering the trimeric IL-1Ra may be determined empirically, and making such determinations is within the skill in the art. Single or multiple dosages may be employed. It is presently believed that an effective dosage or amount of the trimeric IL-1Ra used alone may range from about 1 μg/kg to about 100 mg/kg of body weight or more per day. Interspecies scaling of dosages can be performed in a manner known in the art, e.g., as disclosed in Mordenti, et al., Pharmaceut. Res., 8:1351 (1991). Similar methods may be employed for other therapeutic proteins of the invention.

When in vivo administration of the therapeutic proteins is employed, normal dosage amounts may vary from about 10 ng/kg to up to 100 mg/kg of mammal body weight or more per day, preferably about 1 μg/kg/day to 50 mg/kg/day, depending upon the route of administration. Guidance as to particular dosages and methods of delivery is provided in the literature (see, for example, U.S. Pat. No. 4,657,760; 5,206,344; or 5,225,212). One of skill will appreciate that different formulations will be effective for different treatment compounds and different disorders, that administration targeting one organ or tissue, for example, may necessitate delivery in a manner different from that to another organ or tissue. Those skilled in the art will understand that the dosage of the therapeutic proteins that must be administered will vary depending on, for example, the mammal which will receive the protein, the route of administration, and other drugs or therapies being administered to the mammal.

The trimeric complexes and other therapeutic agents (and one or more other therapies) may be administered concurrently (simultaneously) or sequentially. In particular embodiments, a fusion protein or trimeric complex and a therapeutic agent are administered concurrently. In another embodiment, a fusion protein or trimeric complex is administered prior to administration of a therapeutic agent. In another embodiment, a therapeutic agent is administered prior to a fusion protein or trimeric complex. Following administration, treated cells in vitro can be analyzed. Where there has been in vivo treatment, a treated mammal can be monitored in various ways well known to the skilled practitioner. For instance, serum cytokine responses can be analyzed.

The therapeutic proteins described herein may be used in combination (pre-treatment, post-treatment, or concurrent treatment) with any of one or more TNF inhibitors for the treatment or prevention of the diseases and disorders recited herein, such as but not limited to, all forms of soluble TNF receptors including Etanercept (such as ENBREL®), as well as all forms of monomeric or multimeric p75 and/or p55 TNF receptor molecules and fragments thereof; anti-human TNF antibodies, such as but not limited to, Infliximab (such as REMICADE®), and D2E7 (such as HUMIRA®), and the like. Such TNF inhibitors include compounds and proteins which block in vivo synthesis or extracellular release of TNF. In a specific embodiment, the present invention is directed to the use of an IL-17RA IL-1Ra fusion proteins in combination (pre-treatment, post-treatment, or concurrent treatment) with any of one or more of the following TNF inhibitors: TNF binding proteins (soluble TNF receptor type-I and soluble TNF receptor type-II (“sTNFRs”), as defined herein), anti-TNF antibodies, granulocyte colony stimulating factor; thalidomide; BN 50730; tenidap; E 5531; tiapafant PCA 4248; nimesulide; panavir; rolipram; RP 73401; peptide T; MDL 201,449A; (1R,3 S)-Cis-1-[9-(2,6-diaminopurinyl)]-3-hydroxy-4-cyclopentene hydrochloride; (1R,3R)-trans-1-(9-(2,6-diamino)purine]-3-acetoxycyclopentane; (1R,3R)-trans-1-[9-adenyl)-3-azidocyclopentane hydrochloride and (1R,3R)-trans-1-(6-hydroxy-purin-9-yl)-3-azidocyclo-pentane. TNF binding proteins are disclosed in the art (EP 308 378, EP 422 339, GB 2 218 101, EP 393 438, WO 90/13575, EP 398 327, EP 412 486, WO 91/03553, EP 418 014, JP 127,800/1991, EP 433 900, U.S. Pat. No. 5,136,021, GB 2 246 569, EP 464 533, WO 92/01002, WO 92/13095, WO 92/16221, EP 512 528, EP 526 905, WO 93/07863, EP 568 928, WO 93/21946, WO 93/19777, EP 417 563, WO 94/06476, and PCT International Application No. PCT/US97/12244).

For example, EP 393 438 and EP 422 339 teach the amino acid and nucleic acid sequences of a soluble TNF receptor type I (also known as “sTNFR-I” or “30 kDa TNF inhibitor”) and a soluble TNF receptor type II (also known as “sTNFR-II” or “40 kDa TNF inhibitor”), collectively termed “sTNFRs”, as well as modified forms thereof (e.g., fragments, functional derivatives and variants). EP 393 438 and EP 422 339 also disclose methods for isolating the genes responsible for coding the inhibitors, cloning the gene in suitable vectors and cell types and expressing the gene to produce the inhibitors. Additionally, polyvalent forms (i.e., molecules comprising more than one active moiety) of sTNFR-1 and sTNFR-II have also been disclosed. In one embodiment, the polyvalent form may be constructed by chemically coupling at least one TNF inhibitor and another moiety with any clinically acceptable linker, for example polyethylene glycol (WO 92/16221 and WO 95/34326), by a peptide linker (Neve et al. (1996), Cytokine, 8(5):365-370, by chemically coupling to biotin and then binding to avidin (WO 91/03553) and, finally, by combining chimeric antibody molecules (U.S. Pat. No. 5,116,964, WO 89/09622, WO 91/16437 and EP 315062.

Anti-TNF antibodies include the MAK 195F Fab antibody (Holler et al. (1993), 1st International Symposium on Cytokines in Bone Marrow Transplantation, 147); CDP 571 anti-TNF monoclonal antibody (Rankin et al. (1995), British Journal of Rheumatology, 34:334-342); BAY X 1351 murine anti-tumor necrosis factor monoclonal antibody (Kieft et al. (1995), 7th European Congress of Clinical Microbiology and Infectious Diseases, page 9); CenTNF cA2 anti-TNF monoclonal antibody (Elliott et al. (1994), Lancet, 344:1125-1127 and Elliott et al. (1994), Lancet, 344:1105-1110).

In a particular example, the IL-1Ra fusion proteins described herein may be used in combination with all forms of IL-17 inhibitors (e.g. anti-IL17 receptor antibody, Amgen; anti-IL-17A, anti-IL17F), RORc inhibitors.

In another example, the IL-1Ra fusion proteins described herein may be used in combination with all forms of CD28 inhibitors, such as but not limited to, abatacept (for example ORENCIA®).

In a further example, the IL-1Ra fusion proteins described herein may be used in combination with all forms of IL-6 and/or IL-6 receptor inhibitors, such as but not limited to, Tocilizumab (for example ACTEMRA®).

In a further example, the IL-1Ra fusion proteins described herein may be used in combination with all forms of anti-IL-18 compounds, such as IL-18BP or a derivative, an IL-18 trap, anti-IL-18, anti-IL-18R1, or anti-IL-18RAcP.

In a further example, the IL-1Ra fusion proteins described herein may be used in combination with all forms of anti-IL22, such as anti-IL22 or anti-IL22R.

In a further example, the IL-1Ra fusion proteins described herein may be used in combination with all forms of anti-IL-23 and or IL-12 such as anti-p19, anti-p40 (Ustekinumab), anti-IL-23R.

In a further example, the IL-1Ra fusion proteins described herein may be used in combination with all forms of anti-IL21, such as anti-IL21 or anti-IL21R.

In a further example, the IL-1Ra fusion proteins described herein may be used in combination with all forms of anti-TGF-beta.

In a further example, the IL-1Ra fusion proteins may be used in combination with one or more cytokines, lymphokines, hematopoietic factor(s), and/or an anti-inflammatory agent.

Treatment of the diseases and disorders recited herein can include the use of first line drugs for control of pain and inflammation in combination (pretreatment, post-treatment, or concurrent treatment) with treatment with one or more of the therapuetic proteins provided herein. These drugs are classified as non-steroidal, anti-inflammatory drugs (NSAIDs). Secondary treatments include corticosteroids, slow acting antirheumatic drugs (SAARDs), or disease modifying (DM) drugs, Information regarding the following compounds can be found in The Merck Manual of Diagnosis and Therapy, Sixteenth Edition, Merck, Sharp & Dohme Research Laboratories, Merck & Co., Rahway, N.J. (1992) and in Pharmaprojects, PJB Publications Ltd.

The therapeutic proteins described herein may be used in combination with any of one or more NSAIDs for the treatment of the diseases and disorders recited herein. NSAIDs owe their anti-inflammatory action, at least in part, to the inhibition of prostaglandin synthesis (Goodman and Gilman in “The Pharmacological Basis of Therapeutics,” MacMillan 7th Edition (1985)). NSAIDs can be characterized into at least nine groups: (1) salicylic acid derivatives; (2) propionic acid derivatives; (3) acetic acid derivatives; (4) fenamic acid derivatives; (5) carboxylic acid derivatives; (6) butyric acid derivatives; (7) oxicams; (8) pyrazoles and (9) pyrazolones.

The therapeutic proteins described herein may be used in combination (pretreatment, post-treatment, or concurrent treatment) with any of one or more salicylic acid derivatives, prodrug esters or pharmaceutically acceptable salts thereof. Such salicylic acid derivatives, prodrug esters and pharmaceutically acceptable salts thereof comprise: acetaminosalol, aloxiprin, aspirin, benorylate, bromosaligenin, calcium acetylsalicylate, choline magnesium trisalicylate, magnesium salicylate, choline salicylate, diflusinal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide O-acetic acid, salsalate, sodium salicylate and sulfasalazine. Structurally related salicylic acid derivatives having similar analgesic and anti-inflammatory properties are also intended to be encompassed by this group.

In an additional specific embodiment, the present invention is directed to the use of a therapeutic protein in combination (pretreatment, post-treatment, or concurrent treatment) with any of one or more propionic acid derivatives, prodrug esters or pharmaceutically acceptable salts thereof. The propionic acid derivatives, prodrug esters, and pharmaceutically acceptable salts thereof comprise: alminoprofen, benoxaprofen, bucloxic acid, carprofen, dexindoprofen, fenoprofen, flunoxaprofen, fluprofen, flurbiprofen, furcloprofen, ibuprofen, ibuprofen aluminum, ibuproxam, indoprofen, isoprofen, ketoprofen, loxoprofen, miroprofen, naproxen, naproxen sodium, oxaprozin, piketoprofen, pimeprofen, pirprofen, pranoprofen, protizinic acid, pyridoxiprofen, suprofen, tiaprofenic acid and tioxaprofen. Structurally related propionic acid derivatives having similar analgesic and anti-inflammatory properties are also intended to be encompassed by this group.

In yet another specific embodiment, the present invention is directed to the use of a therapeutic protein in combination (pretreatment, post-treatment, or concurrent treatment) with any of one or more acetic acid derivatives, prodrug esters or pharmaceutically acceptable salts thereof. The acetic acid derivatives, prodrug esters, and pharmaceutically acceptable salts thereof comprise: acemetacin, alclofenac, amfenac, bufexamac, cinmetacin, clopirac, delmetacin, diclofenac potassium, diclofenac sodium, etodolac, felbinac, fenclofenac, fenclorac, fenclozic acid, fentiazac, furofenac, glucametacin, ibufenac, indomethacin, isofezolac, isoxepac, lonazolac, metiazinic acid, oxametacin, oxpinac, pimetacin, proglumetacin, sulindac, talmetacin, tiaramide, tiopinac, tolmetin, tolmetin sodium, zidometacin and zomepirac. Structurally related acetic acid derivatives having similar analgesic and anti-inflammatory properties are also intended to be encompassed by this group.

In another specific embodiment, the present invention is directed to the use of a therapeutic protein in combination (pretreatment, post-treatment, or concurrent treatment) with any of one or more fenamic acid derivatives, prodrug esters or pharmaceutically acceptable salts thereof. The fenamic acid derivatives, prodrug esters and pharmaceutically acceptable salts thereof comprise: enfenamic acid, etofenamate, flufenamic acid, isonixin, meclofenamic acid, meclofenamate sodium, medofenamic acid, mefenamic acid, niflumic acid, talniflumate, terofenamate, tolfenamic acid and ufenamate. Structurally related fenamic acid derivatives having similar analgesic and anti-inflammatory properties are also intended to be encompassed by this group.

In an additional specific embodiment, the present invention is directed to the use of a therapeutic protein in combination (pretreatment, post-treatment, or concurrent treatment) with any of one or more carboxylic acid derivatives, prodrug esters or pharmaceutically acceptable salts thereof. The carboxylic acid derivatives, prodrug esters, and pharmaceutically acceptable salts thereof which can be used comprise: clidanac, diflunisal, flufenisal, inoridine, ketorolac and tinoridine. Structurally related carboxylic acid derivatives having similar analgesic and anti-inflammatory properties are also intended to be encompassed by this group.

In yet another specific embodiment, the present invention is directed to the use of a therapeutic protein in combination (pretreatment, post-treatment, or concurrent treatment) with any of one or more butyric acid derivatives, prodrug esters or pharmaceutically acceptable salts thereof. The butyric acid derivatives, prodrug esters, and pharmaceutically acceptable salts thereof comprise: bumadizon, butibufen, fenbufen and xenbucin. Structurally related butyric acid derivatives having similar analgesic and anti-inflammatory properties are also intended to be encompassed by this group.

In another specific embodiment, the present invention is directed to the use of aa therapeutic protein in combination (pretreatment, post-treatment, or concurrent treatment) with any of one or more oxicams, prodrug esters, or pharmaceutically acceptable salts thereof. The oxicams, prodrug esters, and pharmaceutically acceptable salts thereof comprise: droxicam, enolicam, isoxicam, piroxicam, sudoxicam, tenoxicam and 4-hydroxyl-1,2-benzothiazine 1,1-dioxide 4-(N-phenyl)-carboxamide. Structurally related oxicams having similar analgesic and anti-inflammatory properties are also intended to be encompassed by this group.

In still another specific embodiment, the present invention is directed to the use of a therapeutic protein in combination (pretreatment, post-treatment, or concurrent treatment) with any of one or more pyrazoles, prodrug esters, or pharmaceutically acceptable salts thereof. The pyrazoles, prodrug esters, and pharmaceutically acceptable salts thereof which may be used comprise: difenamizole and epirizole. Structurally related pyrazoles having similar analgesic and anti-inflammatory properties are also intended to be encompassed by this group.

In an additional specific embodiment, the present invention is directed to the use of a therapeutic protein in combination (pretreatment, post-treatment or, concurrent treatment) with any of one or more pyrazolones, prodrug esters, or pharmaceutically acceptable salts thereof. The pyrazolones, prodrug esters and pharmaceutically acceptable salts thereof which may be used comprise: apazone, azapropazone, benzpiperylon, feprazone, mofebutazone, morazone, oxyphenbutazone, phenylbutazone, pipebuzone, propylphenazone, ramifenazone, suxibuzone and thiazolinobutazone. Structurally related pyrazalones having similar analgesic and anti-inflammatory properties are also intended to be encompassed by this group.

In another specific embodiment, the present invention is directed to the use of a therapeutic protein in combination (pretreatment, post-treatment, or concurrent treatment) with any of one or more of the following NSAIDs: .epsilon.-acetamidocaproic acid, S-adenosyl-methionine, 3-amino-4-hydroxybutyric acid, amixetrine, anitrazafen, antrafenine, bendazac, bendazac lysinate, benzydamine, beprozin, broperamole, bucolome, bufezolac, ciproquazone, cloximate, dazidamine, deboxamet, detomidine, difenpiramide, difenpyramide, difisalamine, ditazol, emorfazone, fanetizole mesylate, fenflumizole, floctafenine, flumizole, flunixin, fluproquazone, fopirtoline, fosfosal, guaimesal, guaiazolene, isonixirn, lefetamine HCl, leflunomide, lofemizole, lotifazole, lysin clonixinate, meseclazone, nabumetone, nictindole, nimesulide, orgotein, orpanoxin, oxaceprol, oxapadol, paranyline, perisoxal, perisoxal citrate, pifoxime, piproxen, pirazolac, pirfenidone, proquazone, proxazole, thielavin B, tiflamizole, timegadine, tolectin, tolpadol, tryptamid and those designated by company code number such as 480156S, AA861, AD1590, AFP802, AFP860, AI77B, AP504, AU8001, BPPC, BW540C, CHINOIN 121, CN100, EB382, EL508, F1044, FK-506, GV3658, ITF182, KCNTEI6090, KME4, LA2851, MR714, MR897, MY309, ONO3144, PR823, PV102, PV108, R830, RS2131, SCR152, SH440, SIR133, SPAS510, SQ27239, ST281, SY6001, TA60, TAI-901 (4-benzoyl-1-indancarboxylic acid), TVX2706, U60257, UR2301 and WY41770. Structurally related NSAIDs having similar analgesic and anti-inflammatory properties to the NSAIDs are also intended to be encompassed by this group.

In still another specific embodiment, the present invention is directed to the use of a therapeutic protein in combination (pretreatment, post-treatment or concurrent treatment) with any of one or more corticosteroids, prodrug esters or pharmaceutically acceptable salts thereof for the treatment of the diseases and disorders recited herein, including acute and chronic inflammation such as rheumatic diseases, graft versus host disease and multiple sclerosis. Corticosteroids, prodrug esters and pharmaceutically acceptable salts thereof include hydrocortisone and compounds which are derived from hydrocortisone, such as 21-acetoxypregnenolone, alclomerasone, algestone, amcinonide, beclomethasone, betamethasone, betamethasone valerate, budesonide, chloroprednisone, clobetasol, clobetasol propionate, clobetasone, clobetasone butyrate, clocortolone, cloprednol, corticosterone, cortisone, cortivazol, deflazacon, desonide, desoximerasone, dexamethasone, diflorasone, diflucortolone, difluprednate, enoxolone, fluazacort, flucloronide, flumethasone, flumethasone pivalate, flucinolone acetonide, flunisolide, fluocinonide, fluorocinolone acetonide, fluocortin butyl, fluocortolone, fluocortolone hexanoate, diflucortolone valerate, fluorometholone, fluperolone acetate, fluprednidene acetate, fluprednisolone, flurandenolide, formocortal, halcinonide, halometasone, halopredone acetate, hydro-cortamate, hydrocortisone, hydrocortisone acetate, hydro-cortisone butyrate, hydrocortisone phosphate, hydrocortisone 21-sodium succinate, hydrocortisone tebutate, mazipredone, medrysone, meprednisone, methylprednisolone, mometasone furoate, paramethasone, prednicarbate, prednisolone, prednisolone 21-diedryaminoacetate, prednisolone sodium phosphate, prednisolone sodium succinate, prednisolone sodium 21-m-sulfobenzoate, prednisolone sodium 21-stearoglycolate, prednisolone tebutate, prednisolone 21-trimethylacetate, prednisone, prednival, prednylidene, prednylidene 21-diethylaminoacetate, tixocortol, triamcinolone, triamcinolone acetonide, triamcinolone benetonide and triamcinolone hexacetonide. Structurally related corticosteroids having similar analgesic and anti-inflammatory properties are also intended to be encompassed by this group.

In another specific embodiment, the present invention is directed to the use of a therapeutic protein in combination (pretreatment, post-treatment, or concurrent treatment) with any of one or more slow-acting antirheumatic drugs (SAARDs) or disease modifying antirheumatic drugs (DMARDS), prodrug esters, or pharmaceutically acceptable salts thereof for the treatment of the diseases and disorders recited herein, including acute and chronic inflammation such as rheumatic diseases, graft versus host disease and multiple sclerosis. SAARDs or DMARDS, prodrug esters and pharmaceutically acceptable salts thereof comprise: allocupreide sodium, auranofin, aurothioglucose, aurothioglycanide, azathioprine, brequinar sodium, bucillamine, calcium 3-aurothio-2-propanol-1-sulfonate, chlorambucil, chloroquine, clobuzarit, cuproxoline, cyclo-phosphamide, cyclosporin, dapsone, 15-deoxyspergualin, diacerein, glucosamine, gold salts (e.g., cycloquine gold salt, gold sodium thiomalate, gold sodium thiosulfate), hydroxychloroquine, hydroxychloroquine sulfate, hydroxyurea, kebuzone, levamisole, lobenzarit, melittin, 6-mercaptopurine, methotrexate, mizoribine, mycophenolate mofetil, myoral, nitrogen mustard, D-penicillamine, pyridinol imidazoles such as SKNF86002 and SB203580, rapamycin, thiols, thymopoietin and vincristine. Structurally related SAARDs or DMARDs having similar analgesic and anti-inflammatory properties are also intended to be encompassed by this group.

In another specific embodiment, the present invention is directed to the use of a therapeutic protein in combination (pretreatment, post-treatment, or concurrent treatment) with any of one or more COX2 inhibitors, prodrug esters or pharmaceutically acceptable salts thereof for the treatment of the diseases and disorders recited herein, including acute and chronic inflammation. Examples of COX2 inhibitors, prodrug esters or pharmaceutically acceptable salts thereof include, for example, celecoxib. Structurally related COX2 inhibitors having similar analgesic and anti-inflammatory properties are also intended to be encompassed by this group. Examples of COX-2 selective inhibitors include but not limited to etoricoxib, valdecoxib, celecoxib, licofelone, lumiracoxib, rofecoxib, and the like.

In still another specific embodiment, the present invention is directed to the use of a therapeutic protein in combination (pretreatment, post-treatment, or concurrent treatment) with any of one or more antimicrobials, prodrug esters or pharmaceutically acceptable salts thereof for the treatment of the diseases and disorders recited herein, including acute and chronic inflammation. Antimicrobials include, for example, the broad classes of penicillins, cephalosporins and other beta-lactams, aminoglycosides, azoles, quinolones, macrolides, rifamycins, tetracyclines, sulfonamides, lincosamides and polymyxins. The penicillins include, but are not limited to penicillin G, penicillin V, methicillin, nafcillin, oxacillin, cloxacillin, dicloxacillin, floxacillin, ampicillin, ampicillin/sulbactam, amoxicillin, amoxicillin/clavulanate, hetacillin, cyclacillin, bacampicillin, carbenicillin, carbenicillin indanyl, ticarcillin, ticarcillin/clavulanate, azlocillin, meziocillin, peperacillin, and mecillinam. The cephalosporins and other beta-lactams include, but are not limited to cephalothin, cephapirin, cephalexin, cephradine, cefazolin, cefadroxil, cefaclor, cefamandole, cefotetan, cefoxitin, ceruroxime, cefonicid, ceforadine, cefixime, cefotaxime, moxalactam, ceftizoxime, cetriaxone, cephoperazone, ceftazidime, imipenem and aztreonam. The aminoglycosides include, but are not limited to streptomycin, gentamicin, tobramycin, amikacin, netilmicin, kanamycin and neomycin. The azoles include, but are not limited to fluconazole. The quinolones include, but are not limited to nalidixic acid, norfloxacin, enoxacin, ciprofloxacin, ofloxacin, sparfloxacin and temafloxacin. The macrolides include, but are not limited to erythomycin, spiramycin and azithromycin. The rifamycins include, but are not limited to rifampin. The tetracyclines include, but are not limited to spicycline, chlortetracycline, clomocycline, demeclocycline, deoxycycline, guamecycline, lymecycline, meclocycline, methacycline, minocycline, oxytetracycline, penimepicycline, pipacycline, rolitetracycline, sancycline, senociclin and tetracycline. The sulfonamides include, but are not limited to sulfanilamide, sulfamethoxazole, sulfacetamide, sulfadiazine, sulfisoxazole and co-trimoxazole (trimethoprim/sulfamethoxazole). The lincosamides include, but are not limited to clindamycin and lincomycin. The polymyxins (polypeptides) include, but are not limited to polymyxin B and colistin.

It should be noted that the section headings are used herein for organizational purposes only, and are not to be construed as in any way limiting the subject matter described. All references cited herein are incorporated by reference in their entirety for all purposes.

The Examples that follow are merely illustrative of certain embodiments of the invention, and are not to be taken as limiting the invention, which is defined by the appended claims.

### EXAMPLES

### Example 1

### Format, Production and Purification of Trimeric IL-1Ra

It has been previously been shown that IL-1Ra can be produced as recombinant protein in E. coli. (Steinkasserer et al 1992. FEBS 310:63-65). The protein is very stable and refolds efficiently. Isoforms of IL-1Ra with additional amino acids in the N-terminal have been also described (Haskill et al 1991, PNAS 88:3681-3685; Muzio et al 1995, JEM 182, 623-628)). These molecules bind IL-1R as well as the mature secreted form indicating that it is possible to fuse extra peptide to the N-terminal of the antagonist without compromising the binding to the receptor. Crystal structure analysis of IL-1Ra interaction with IL-1R also supports that N-terminal alterations do not affect interactions with IL1R (Sclireuder et al 1997, Nature 386: 190-194). IL-1Ra was cloned from a human cDNA library derived from bone marrow and/or human placenta.

Trimeric IL-1Ra was designed as a C-terminal fusion to the Trip-trimerization unit. Eight different fusion proteins were designed, four with full length trimerization units (Trip) and four with a nine amino acid truncation of the trimerization unit (I10Trip). IL-1ra was than fused with either trimerization unit using four different C-terminal fusions. C-terminal variations termed Trip V, Trip T, Trip Q and Trip K allow for unique presentation of the CTLD domains on the trimerization domain. The Trip K variant is the longest construct and contains the longest and most flexible linker between the CTLD and the trimerization domain. Trip V, Trip T, Trip Q represent fusions of the CTLD molecule directly onto the trimerization module without any structural flexibility but are turning the CTLD molecule ⅓rd going from Trip V to Trip T and from Trip T to Trip Q. This is due to the fact that each of these amino acids is in an α-helical turn and 3.2 aa are needed for a full turn

The following proteins were produced as the following Granzyme B cleavable fusion proteins in BL21 AI bacteria. The underlined portions denotes the trimerization unit, and the bold part denotes the IL-1Ra part:

CII-H6-GrB-GG-TripK-IL-1ra:

(SEQ ID NO: 34)

MVRANKRNEALRIESALLNKIAMLGTEKTAEGGSHHHHHHGSIEPDGGEG
PTQKPKKIVNAKKDVVNTKMFEELKSRLDTLAQEVALLKEQQALQTVSLK
RPSGRKSSKMQAFRIWDVNQKTFYLRNNQLVAGYLQGPNVNLEEKIDVVP
IEPHALFLGIHGGKMCLSCVKSGDETRLQLEAVNITDLSENRKQDKRFAF
IRSDSGPTTSFESAACPGWFLCTAMEADQPVSLTNMPDEGVMVTKFYFQE
DE;
CII-H6-GrB-GG-TripV-IL-1ra:

(SEQ ID NO: 35)

MVRANKRNEALRIESALLNKIAMLGTEKTAEGGSHHHHHHGSIEPDGGEG
PTQKPKKIVNAKKDVVNTKMFEELKSRLDTLAQEVALLKEQQALQTVRPS
GRKSSKMQAFRIWDVNQKTFYLRNNQLVAGYLQGPNVNLEEKIDVVPIEP
HALFLGIHGGKMCLSCVKSGDETRLQLEAVNITDLSENRKQDKRFAFIRS
DSGPTTSFESAACPGWFLCTAMEADQPVSLTNMPDEGVMVTKFYFQEDE;
CII-H6-GrB-GG-TripT-IL-1ra:

(SEQ ID NO: 36)

MVRANKRNEALRIESALLNKIAMLGTEKTAEGGSHHHHHHGSIEPDGGEG
PTQKPKKIVNAKKDVVNTKMFEELKSRIDTLAQEVALLKEQQALQTRPSG
RKSSKMQAFRIWDVNQKTFYLRNNQLVAGYLQGPNVNLEEKIDVVPIEPH
ALFLGIHGGKMCLSCVKSGDETRLQLEAVNITDLSENRKQDKRFAFIRSD
SGPTTSFESAACPGWFLCTAMEADQPVSLTNMPDEGVMVTKFYFQEDE;
CII-H6-GrB-GG-TripQ-IL-1ra:

(SEQ ID NO: 37)

MVRANKRNEALRIESALLNKIAMLGTEKTAEGGSHHHHHHGSIEPDGGEG
PTQKPKKIVNAKKDVVNTKMFEELKSRLDTLAQEVALLKEQQALQRPSGR
KSSKMQAFRIWDVNQKTFYLRNNQLVAGYLQGPNYNLEEKIDVVPIEPHA
LFLGIHGGKMCLSCVKSGDETRLQLEAVNITDLSENRKQDKRFAFIRSDS
GPTTSFESAACPGWFLCTAMEADQPVSLTNMPDEGYMVTKFYFQEDE;
CII-H6-GrB-I10-TripK-IL-1ra

(SEQ ID NO: 38)

MVRANKRNEALRIESALLNKIAMLGTEKTAEGGSHHHHHHGSIEPDIVNA
KKDVVNTKMFEELKSRLDTLAQEVALLKEQQALQTVSLKRPSGRKSSKMQ
AFRIWDVNQKTFYLRNNQLYAGYLQGPNVNLEEKIDVVPIEPHALFLGIH
GGKMCLSCVKSGDETRLQLEAVNITDLSENRKQDKRFAFIRSDSGPTTSF
ESAACPGWFLCTAMEADQPVSLTNMPDEGVMVTKFYFQEDE;
CII-H6-GrB-I10-TripV-IL-1ra

(SEQ ID NO: 39)

MVRANKRNEALRIESALLNKIAMLGTEKTAEGGSHHHHHHGSIEPDIVNA
KKDVVNTKMFEELKSRLDTLAQEVALLKEQQALQTVRPSGRKSSKMQAFR
IWDVNQKTFYLRNNQLVAGYLQGPNVNLEEKIDVVPIEPHALFLGIHGGK
MCLSCVKSGDETRLQLEAVNITDLSENRKQDKRFAFIRSDSGPTTSFESA
ACPGWFLCTAMEADQPVSLTNMPDEGVMVTKFYFQEDE;
CII-H6-GrB-I10-TripT-IL-1ra:

(SEQ ID NO: 40)

MVRANKRNEALRIESALLNKIAMLGTEKTAEGGSHHHHHHGSIEPDIVNA
KKDVVNTKMFEELKSRLDTLAQEVALLKEQQALQTRPSGRKSSKMQAFRI
WDVNQKTFYLRNNQLVAGYLQGPNVNLEEKIDVVPIEPHALFLGIHGGKM
CLSCVKSGDETRLQLEAVNITDLSENRKQDKRFAFIRSDSGPTTSFESAA
CPGWFLCTAMEADQPVSLTNMPDEGVMVTKFYFQEDE;
and
CII-H6-GrB-I10-TripQ-IL-1ra:

(SEQ ID NO: 41)

MVRANKRNEALRIESALLNKIAMLGTEKTAEGGSHHHHHHGSIEPDIVNA
KKDVVNTKMFEELKSRLDTLAQEVALLKEQQALQRPSGRKSSKMQAFRIW
DVNQKTFYLRNNQLVAGYLQGPNVNLEEKIDVVPIEPHALFLGIHGGKMC
LSCVKSGDETRLQLEAVNITDLSENRKQDKRFAFIRSDSGPTTSFESAAC
PGWFLCTAMEADQPVSLTNMPDEGVMYTKFYFQEDE

All constructs were captured on NiNTA Superflow (Qiagen), refolded and further purified on SP-Sepharose FF (GE Heathcare). From expression in shake flask or from a fermentation of the trimeric IL1-Ra, inclusion bodies were purified. Packed cell pellet was homogenized in lysis-buffer (50 mM Tris-HCl, pH 8.0, 25 w/v % Sucrose, 1 mM EDTA) by sonication (50 g wet cell pellet per 100 mL lysis buffer). Then 100 mg lysozyme per 100 mL lysis-buffer was added and mixed before the sample was left for 15 min at R.T. The sample was then sonicated for 2-5 min with mixing in between. Detergent buffer (0.2 M NaCl, 1 w/v % Deoxycholate, Na salt, 1 w/v % Nonidet P40, 20 mM Tris-HCl, pH 7.5, 2 mM EDTA) was added and the sample is mixed and sonified again. The inclusion bodies were recovered by centrifugation for 25 min at 8.000 rpm, 4° C. The supernatant was stored at 4° C. and the pellet resuspended in 100 mL TRITON® X-100 buffer (0.5 w/v % TRITON® X-100, 1 mM EDTA, pH 8) per 50 g original cell pellet. Inclusion bodies were recovered by centrifugation for 25 min at 8.000 rpm, 4° C. and the supernatant was stored at 4° C. The TRITON® X-100 buffer wash is repeated once more and the inclusion bodies were recovered by centrifugation for 5 min at 12.000 rpm, 4° C.

The inclusion bodies were re-suspended in 30 mL denaturing buffer/gram original cell paste (6 M urea, 10 mM EDTA, 20 mM Tris/HCl and 20 mM β-Mercaptoethanol, pH 8.0) at 28° C. for 2 h. The suspension was centrifuged at 7500 g for 15 min to remove insoluble material. Following this CaCl2 was added to 20 mM final concentration and the solution was applied to a 100 mL Ni-NTA Superflow column equillibrated in NTA buffer (8 M Urea; 1000 mM NaCl; 50 mM Tris HCl pH 8.0; 5 mM β-Mercaptoethanol) and washed until a stable baseline was obtained. A further wash with 250 mL guanidine-HCl, 50 mM Tris-HCl pH 8.0, 5 mM β-Mercaptoethanol followed by wash with 100 mL buffer NTA.

Two refolding methods have been used, dialysis refolding and on-column refolding and both have yielded pure and soluble protein. For dialysis refolding the resuspended inclusion bodies was used directly for dialysis over into 1×PBS containing 3 M urea, 1 mM EDTA, pH 7.2 over night. The day after the dialysis was continued into 1×PBS containing 0 M urea, 1 mM EDTA, pH 7.2.

For on-column refolding the washed Ni-NTA Superflow column with protein bound, the resin was washed with 4 CV ml 1×PBS containing 3 M urea, pH 7.2 before a linear gradient of 10 CV 1×PBS containing 3 M urea, pH 7.2 and 10 CV 1×PBS containing 0 M urea, pH 7.2 was run. To recover the refolded trimeric IL-1ra, the column eluted with 1×PBS, 10 mM EDTA, pH 6.0 and fraction were collected.

Following refolding cleavage with recombinant human Granzyme B was performed by adjusting the pH in the eluate to 7.5 with NaOH before Granzyme B was added at a 1:500 ratio (granzyme/protein) and incubated at 25° C. over night. The progress was followed by SDS-PAGE.

Finally, the cleaved protein was purified using SP-Sepharose FF (GE Healthcare) cation exchange step. A ˜50 mL SP-Sepharose FF was packed and equilibrated in buffer A (1×PBS, 1 mM EDTA pH 5,5) until stabile basis line was obtained. The cleavage reaction was diluted 1:3 with buffer A and loaded on the column followed by a wash in buffer A until stabile basis line was monitored. A gradient from 10 CV buffer A to 10 CV Buffer B (1×PBS, 1 mM EDTA+0.5 M NaCl pH 5,5) was setup and fractions collected in 5 mL. Protein containing fractions were analyzed on SDS-PAGE before pooling the protein product.

Alternatively, the supernatant from the above inclusion body preparation were used to purify the protein. The soluble Trimeric IL1-ra in the supernatant was purified on Ni-NTA Superflow (Qiagen) column equilibrated in Buffer A (20 mM TrisHCL, 50 mM NaCl pH 8.0. A pool was made of the washes from the inclusion body purification and it was centrifuged at 10000 rpm for 10 min before CaCl2 was added to 5 mM and Tris-HCl to 20 mM and the pH adjusted to 6.0 with HCl/NaOH. The pool was loaded on the column and washed in buffer A until stabile basis line. Following a wash in buffer A+1 M NaCl until stabile basis line, the bound protein was eluted with buffer A+20 mM EDTA and fractions were collected. Hereafter the protein pool was cleaved with Granzyme B and polished on a SP-Sepharose FF column as described above. The soluble fraction of CII-H6-GrB-GG-TripK-IL-1ra from 3 L expression culture gave a final yield of 95 mg of TripK-IL-1Ra following (˜250 mg CII-H6-GrB-TripK-IL-1Ra after capture Ni-NTA Superflow (Qiagen). Since the yield and purity of the protein from the soluble fraction was significantly better than doing refolding, this path was chosen following the initial construct testing.

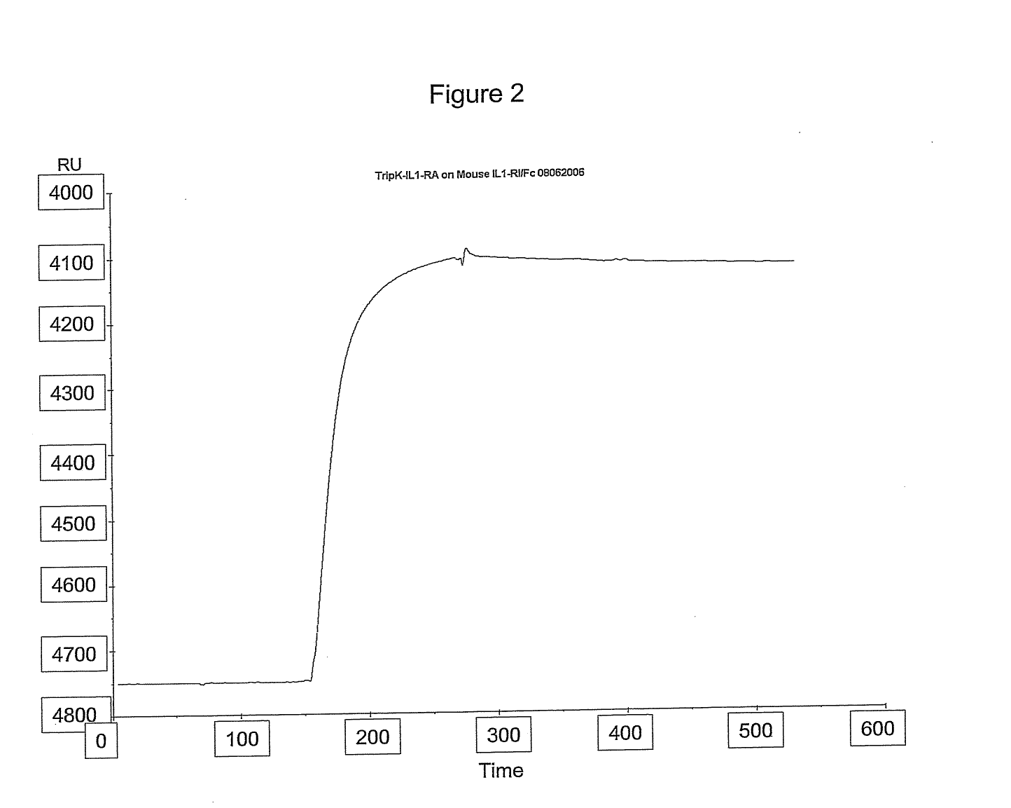

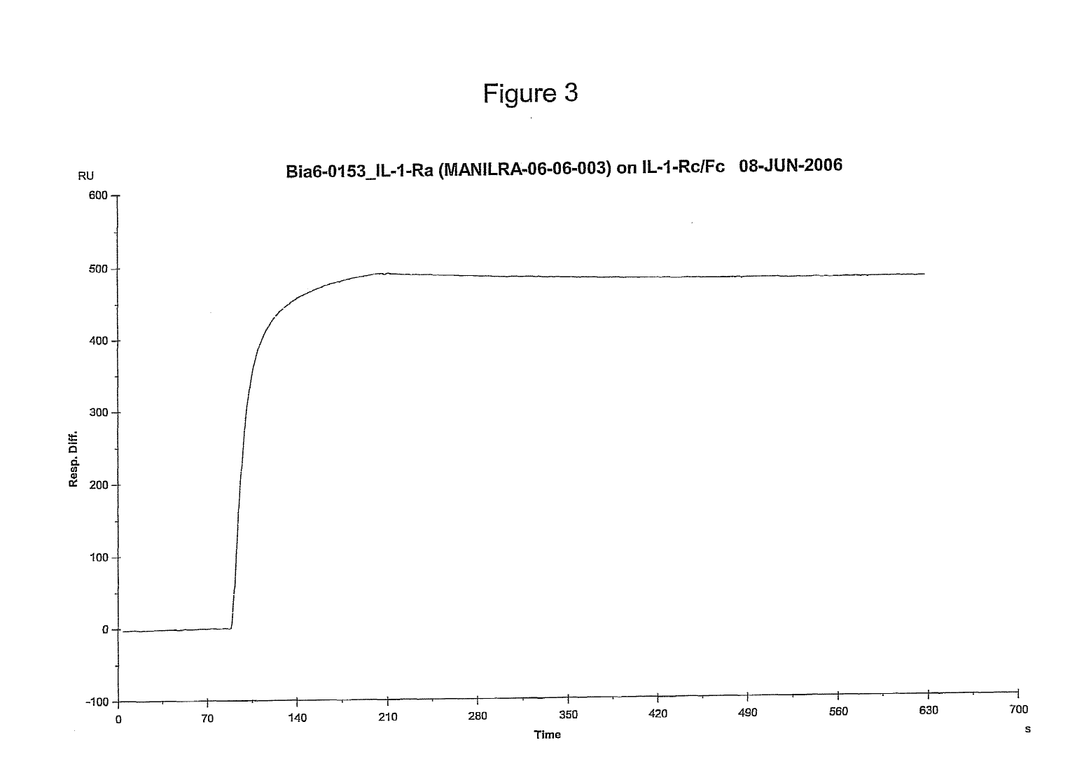

The ability of the refolded protein to bind to IL-1 Receptor 1 was analyzed on a Biacore 3000 (Biacore, Uppsala, Sweden) where mouse IL1-R1/Fc was coupled to CM5 sensor chips and binding of soluble TripK-IL-1ra to IL-1RI protein was measured. Results of uncleaved CII-H6-GrB-TripK-IL-1ra refolding by dialysis are shown in FIG. 2 and uncleaved CII-H6-GrB-TripK-IL-1ra on-column NiNTA refolding is shown in FIG. 3. The cleavage and purification assays produced the trimeric IL-1Ra compounds of SEQ ID NOs: 47-54.

### Example 2

### Trimeric IL-1Ra Compounds Ability to Inhibit IL-1 Induction of IL-8 in U937 Cells

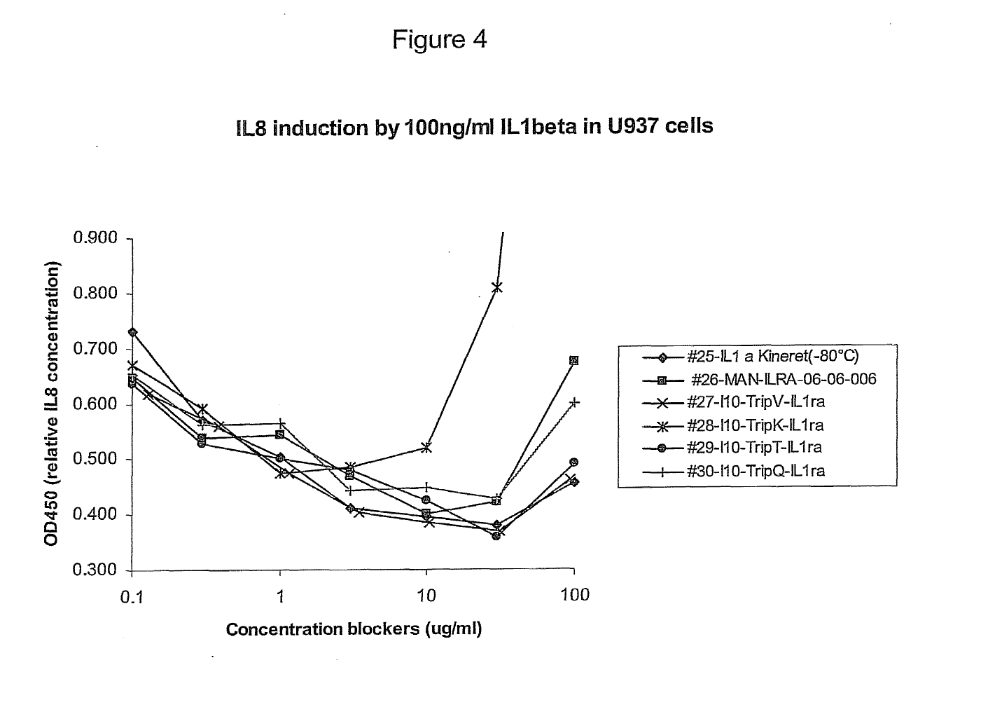

GG-TripV-IL-1ra (Trip V-IL1Ra), GG-TripK-IL-1ra (Trip K-IL1Ra), GG-TripT-IL-1ra (Trip T-IL1Ra) and CII-H6-GrB-GG-TripT-IL-1ra (Trip Q-IL1Ra) were further analysed for their ability to inhibit IL-1 induction of IL-8 in U937 cells. Results are shown in FIG. 4.

The compounds are essentially equally effective in blocking the response and they appear all to be as effective as KINERET® (when compared on w/w). Due to buffer effects in the assay, at the highest protein concentration used (100 μg/mL) IL-8 production increases instead of further decreasing. Based on several in vitro efficacy assays as well as Biacore assays, it was determined that TripT IL1Ra was the best compound based on blocking and binding efficacy as well as production yields.

### Example 3

### Pegylated Trimeric IL-1Ra Compounds

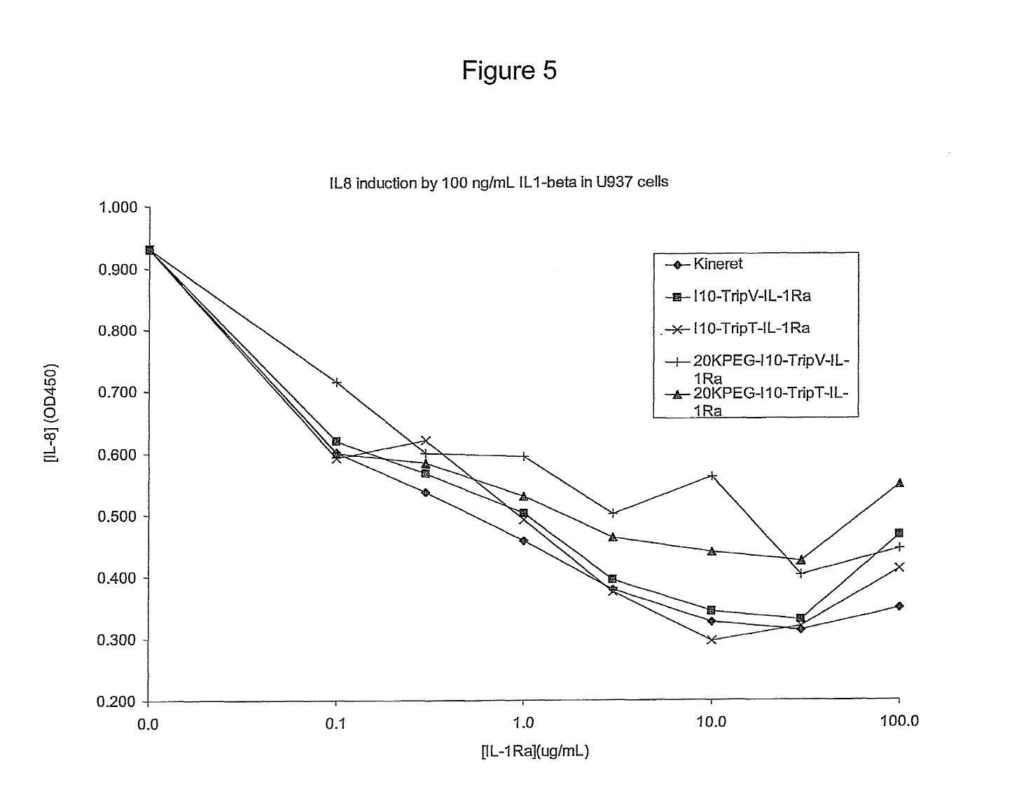

Since the in vivo half life is a crucial parameter in the efficacy of KINERET® (KINERET® has only a half life in humans of 4-6 hours and has therefore, to be applied once daily) the ability to pegylate the TripT IL1Ra by N-terminal pegylation was tested. The trimeric IL1-Ra is pegylated at the N terminus. Trimeric IL1-Ra antagonist proteins after the final step of the purification procedure described above were used as starting point for pegylations. The proteins were buffer changed into PBS buffer pH 6.0 for the pegylation reaction. The protein concentration in the reaction was between 0.5 and 3.5 mg/mL and a 5-10 molar excess of mPeg5K-Aldehyde or mPeg20K-Aldehyde (Nektar) supplemented with 20 mM cyanoborohydride (NaCNBH3) was used. The reaction was carried out at 20° C. for 16 hours. Following the reaction mixture was applied to Source 15S column (GE Healtcare) to purify the monopegylated form. As shown in FIG. 5, antagonistic activity of the pegylated version was reduced compared to the unpegylated protein. However, the pegylated protein still has good IL1 blocking efficacy.

### Example 4

### Pharmacokinetic Analysis of Trimeric IL1Ra Proteins in Male Lewis Rats after i.v. Injection

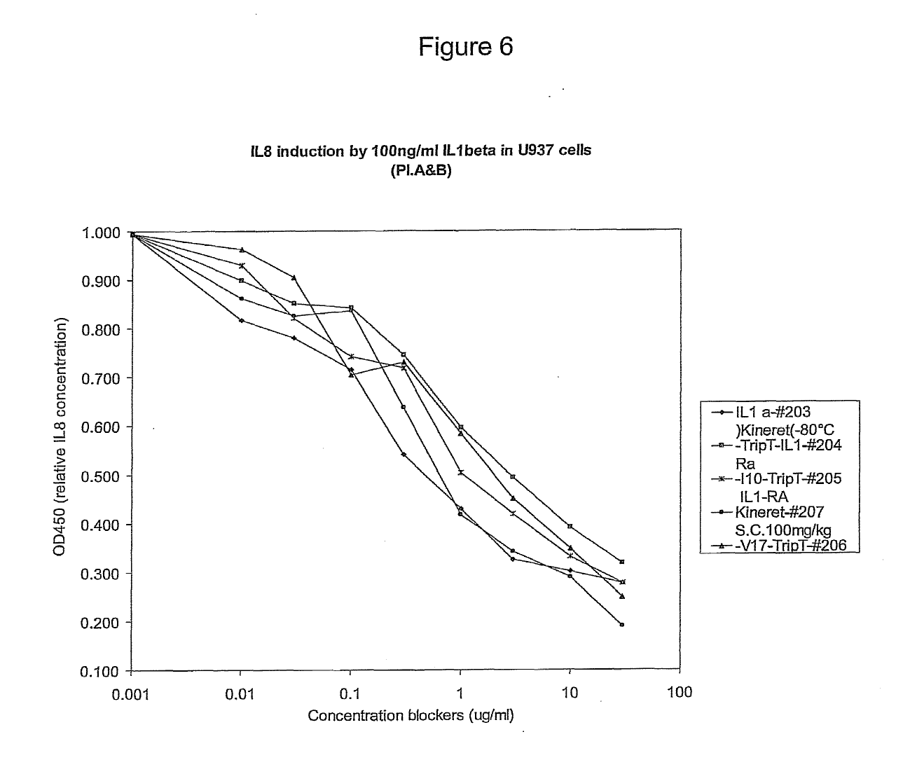

Three of the trimeric IL1Ra polypeptides described in the previous examples were chosen for pharmacokinetic analysis. The differences in the constructs were in the N-terminus of the trimerization domain: full length (FL), first nine amino acids truncated (I10) and the first 16 amino acids truncated (V17). The 10 construct represents a naturally occurring deletion variant of the trimerization domain and lacks the O-glycosylation site at Thr 4. The V17 derivative represents a deletion of the first exon encoding the trimerization domain and lacks a characterized heparin binding site. This site is also partially removed in the I10 construct. In vitro efficacy of the IL-1Ra molecules was verified in a U937 cell assay as shown in FIG. 6.

The pharmacokinetic profile of these three constructs polypeptides were analysed in Lewis rats after intravenous (i.v.) injections. The profiles obtained were compared to the pharmacokinetic profile of KINERET® in the same experiment. The pharmacokinetic study was conducted using four male Lewis rats per group, and the constructs that were used were FL IL-1Ra, I10 IL-1Ra, V17 IL-1Ra and KINERET®, Single i.v. doses of 100 mg/kg were given to the animals. The test compound was dissolved in vehicle (4.4 mM NaCitrate, pH 6.5, 93.8 mM NaCl, 0.33 mM EDTA, 0.7 g TWEEN®-80) and administered through the tail vein (vena sacralis media) or the hind paw vein (vena saphena).

Blood was then collected from four animals per time-point at baseline (zero hours) and 0.5, 1, 2, 4, 8, 12, 24, 48, 72 h post dosing. Blood samples of approximately 100 μl were collected from the tip of the tails in Microtainers™. Plasma was collected and transferred into polypropylene tubes. Plasma samples were then stored at <−70° C. until measurements were performed. Animals were then sacrificed by CO₂ inhalation and the carcasses were discarded without pathological examination. The IL-1Ra compound levels and KINERET® levels in plasma were then determined by ELISA.

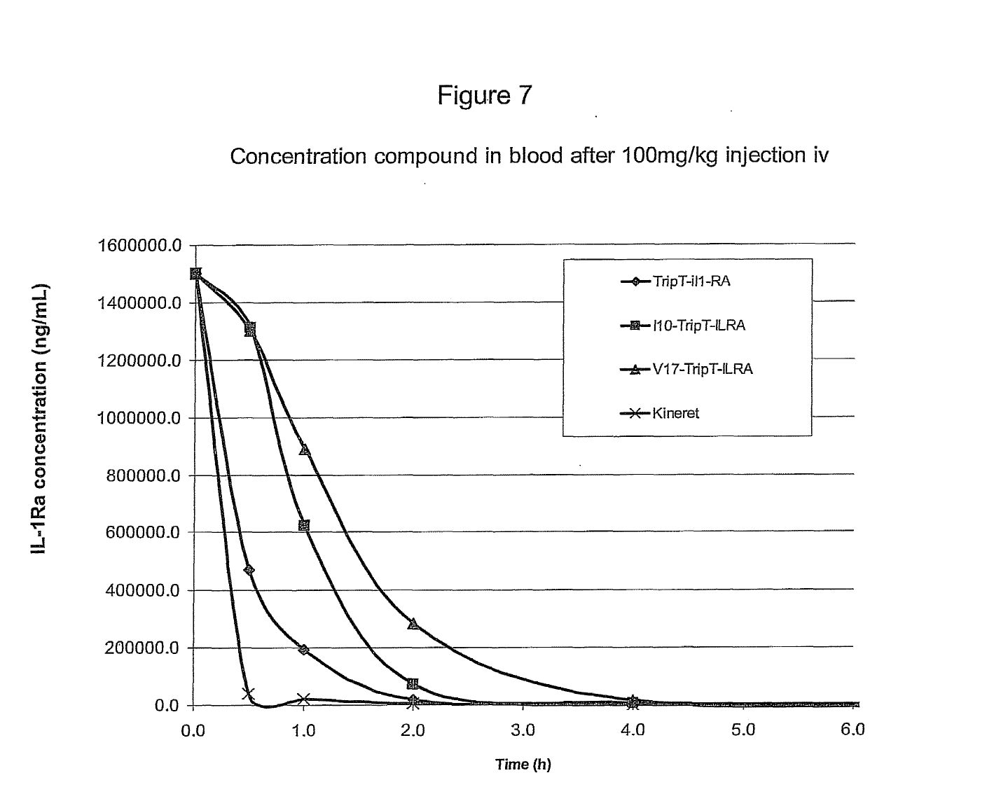

The average body weight of each rat was 250 grams. Assuming that the rat average blood volume was 16.5 mL a theoretical maximum initial concentration of the compounds of 1,500,000 ng/mL was calculated after i.v. injection. These concentrations are shown in FIG. 7. This starting level was used as starting value for the analysis. No observations of side effects or changes in animal well being were observed.

Following blood sampling at the above indicated time points, an ELISA assay was used to measure the injected protein in the blood samples. Based on these ELISA results, area under the curve (AUC) was used as a measure of drug exposure and the plasma half life were calculated using standard software. The areas under the curve are shown in Table 2 and the plasma half lives of the proteins are shown in Table 3.

TABLE 2

| | | AUC protein/ |
|---|---|---|
| Protein | AUC (ng/mL * h) | AUC KINERET ® |
| FL IL1Ra | 809292 | 1.89 |
| I10 IL1Ra | 1637866 | 3.82 |
| V17 IL1Ra | 2177781 | 5.08 |
| KINERET ® | 428414 | 1 |

TABLE 3

| | | Half life protein/ |
|---|---|---|
| Protein | Half life (min.) | Half life KINERET ® |
| FL IL1Ra | 20 | 17 |
| I10 IL1Ra | 54 | 45 |
| V17 IL1Ra | 69 | 58 |
| KINERET ® | 1.2 | 1 |

These i.v. data indicate that the trimeric compounds have superior plasma half lives in comparison to KINERET®. The half life of KINERET® is about 1.2 minutes, whereas the half life of the V17 IL1Ra trimeric protein after i.v. injection is about 69 minutes. Dependent on the criteria used in the analysis the relative increase in AUC is between two-fold for FL IL1Ra trimer and five-fold for V17 IL1Ra trimer, indicating substantially improved drug exposure using the trimerized variants compared to KINERET®.

### Example 5

### Production of Met-I10-TripT-IL1ra and GG-V17-TripT-IL1ra and Rat CIA Model

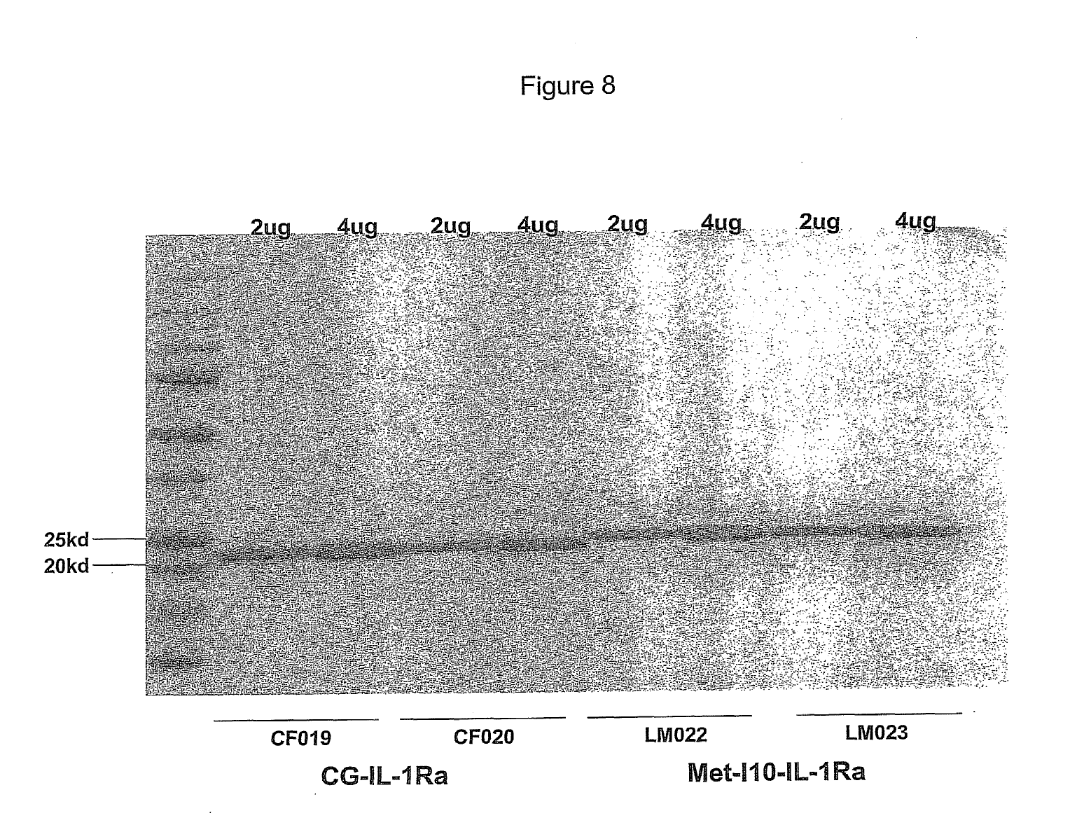

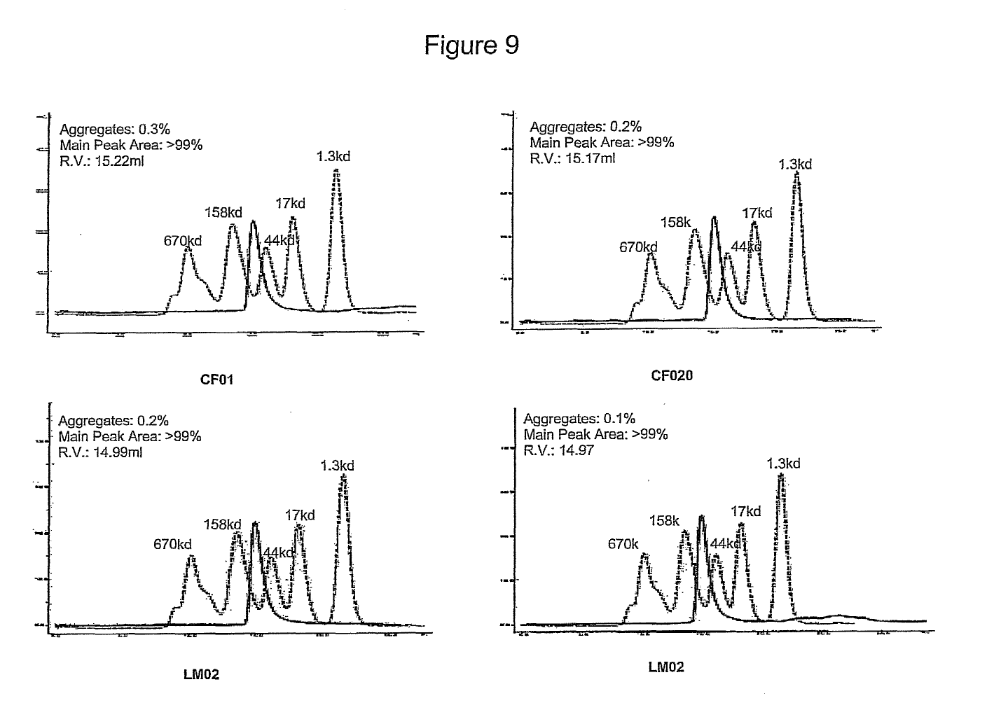

Both molecules were produced by BL21 AI bacteria in 10 L fermentor runs using either 2×TY medium (Met-I10-TripT-IL-1Ra) or chemically defined minimal medium (GG-V17-TripT-IL-1Ra). Cell pellets were obtained by centrifugation at 5887×g for 20 min, then resuspended in 10 mM Na₂HPO₄ pH 6. For Met-I10-TripT-IL-1ra, the soluble cell fraction containing the protein of interest was obtained by high pressure homogenization (2×17.000 psi) followed by 10 min centrifugation at 10.000×g. The supernatant was diluted with 10 mM Na₂HPO₄ pH 7.4 and run over a SP-Sepharose FF column (cation exchange, GE Healthcare) followed by Q-Sepharose FF (anion exchange. GE Healthcare) using an AKTA fPLC. In a last step, proteins were run through a Mustang E filter (Pall) to remove endotoxin, followed by buffer exchange into PBS pH 7.4 and concentration to 50 mg/mL. The GG-V17-TripT-IL-1Ra protein was expressed as a fusion protein comprising an N-terminal booster domain, phage CII protein, followed by a human Granzyme B cleavage site. The GG-V17-TripT-IL-1Ra was purified from fermentation cell pellets by homogenization in lysis buffer containing lysozyme followed by centrifugation for 25 min at 8000 rpm. The supernatant was then run through a FRACTOGEL® EMD Chelate (M) column (EMD Chemicals Inc.), and the eluate was buffer exchanged into 20 mM Tris pH 7.5, 150 mM NaCl. The protein fraction was then digested with recombinant human Granzyme B (made in house, ref to patent). After dilution with PBS pH 6, the proteins were purified using SP Sepharose FF followed by Mustang E filtration and FRACTOGEL® EMD Chelate (M) column in flow through mode to remove the fusion tag and human Granzyme B. Final, the protein was buffer exchanged into PBS pH 7.4 and concentrated to 50 mg/mL. Yields for both Met-I10-TripT-IL-1ra and GG-V17-TripT-IL-1ra proteins were 3-5 g/L, purity >95% as determined by SDS-PAGE (FIG. 8), RP-HPLC and MS. Endotoxin levels were <3EU/mg as determined using a LAL assay (Lonza). Aggregates were <0.5% as determined by analytical SEC (FIG. 9) and host cell protein <6 ng/mL. Two batches (LM022, LM023) of Met-I10-TripT-IL-1 ra and two batches (CF019, CF020) of GG-V17-TripT-IL-1ra were tested in above assays.

Female Lewis rats with 4-day established type II collagen arthritis were treated subcutaneously (SC), daily (QD) on arthritis days 1-3 with Vehicle (10 mM phosphate buffer pH 7.4), or equimolar amounts of IL-1ra administering either monomeric IL-1ra (100 mg/kg KINERET®), or trimerized IL1ra (120 mg/kg Met-I10-TripT-IL1ra, or 120 mg/kg GG-V17-TripT-IL1ra). In order to have only one set of controls, all rats in the QD groups were dosed with the respective vehicle (10 mM phosphate buffer pH 7.4, or sodium citrate buffer pH 6.5 for KINERET®) at the 2nd and 3rd dosings to keep manipulations constant. Animals were terminated on arthritis day 4. Efficacy evaluation was based on ankle caliper measurements, expressed as area under the curve (AUC), terminal hind paw weights and body weights (Bendele et al 2000, Arthritis+Rheumatism 43:2648-2659). All animals survived to study termination. Rats injected with KINERET® or its vehicle (CSEP) vocalized during the injection process thus suggesting that subcutaneous irritation was occurring. No vocalization occurred with any other injections.

Animals (8/group for arthritis, 4/group for normal), housed 4/cage, were anesthetized with Isoflurane and received subcutaneous/intradermal (SC/ID) injections with 300 μl of Freund's Incomplete Adjuvant (Difco, Detroit, Mich.) containing 2 mg/ml bovine type II collagen (Elastin Products, Owensville, Mo.) at the base of the tail and 2 sites on the back on days 0 and 6. Dosing by subcutaneous route (QD at 24 hour intervals) was initiated on arthritis day 1 and continued through day 3. Experimental groups were as shown in Table 4

TABLE 4

| | | QD SC Treatment 2.3 ml/kg, days 1-3, |
|---|---|---|
| Group | N | Dose volumes are based on equivalent IL-1ra molecules |
| 1 | 4 | Normal controls, vehicle (10 mM phosphate buffer pH 7.4) |
| | | TID |
| 2 | 8 | Arthritis+ KINERET ® QD (100 mg/kg), vehicle |
| | | (sodium citrate buffer pH 6.5) at other times |
| 3 | 8 | Arthritis+ Met-I10-TripT-IL1ra QD (120 mg/kg), vehicle |
| | | (10 mM phosphate buffer pH 7.4) at other times |
| 4 | 8 | Arthritis+ V17-TripT-IL1ra QD (120 mg/kg), vehicle |
| | | (10 mM phosphate buffer pH 7.4) at other times |

Rats were weighed on days 0-4 of arthritis, and caliper measurements of ankles were taken every day beginning on day 0 of arthritis (study day 9). After final body weight measurement, animals were euthanized, and hind paws were transected at the level of the medial and lateral malleolus and weighed (paired).

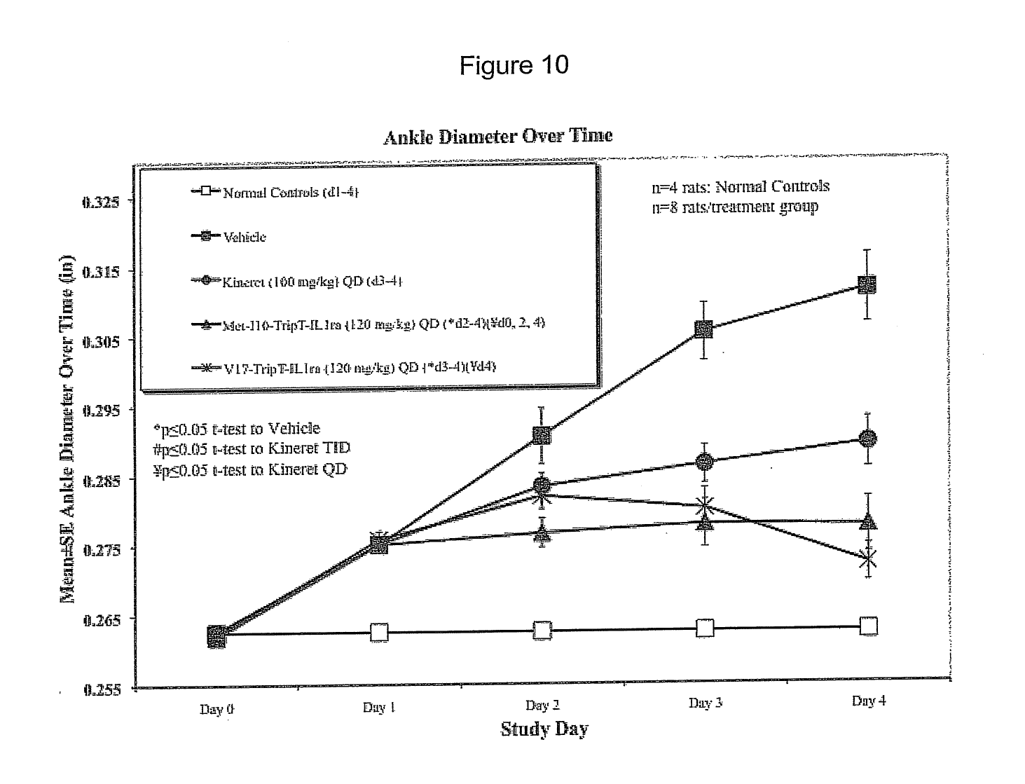

Significant reduction of ankle diameter was seen in rats treated with 100 mg/kg KINERET® QD (d3-4), 120 mg/kg Met-I10-TripT-IL1ra QD (d2-4), or 120 mg/kg GG-V17-TripT-IL1ra QD (d3-4), as compared to vehicle treated disease control animals. Reduction of ankle diameter AUC was significant for rats treated with 100 mg/kg KINERET® QD (34%), 120 mg/kg Met-I10-TripT-IL1ra QD (54%), or 120 mg/kg GG-V17-TripT-IL1ra QD (49%), as compared to vehicle treated disease control animals. Met-I10-TripT-IL1ra QD treatment resulted in significantly reduced anlcle diameter AUC compared to KINERET® QD treatment (p<0.035 at the end of the study). Also, GG-V17-TripT-IL1ra QD treatment resulted in significantly reduced ankle diameter AUC compared to KINERET® QD treatment at the end of the study (p<0.001). (FIG. 10)

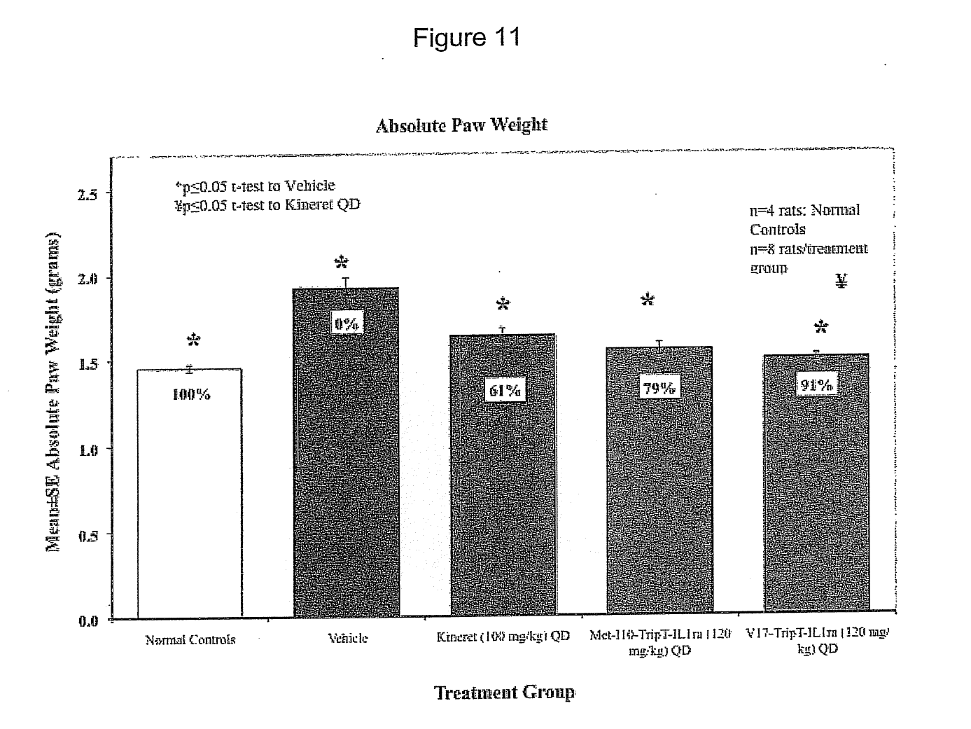

Reduction of final paw weight was significant for rats treated with 100 mg/kg KINERET® QD (61%), 120 mg/kg Met-I10-TripT-IL1ra QD (79%), or 120 mg/kg GG-V17-TripT-IL1ra QD (91%), as compared to vehicle treated disease control animals. GG-V17-TripT-IL1ra QD treatment resulted in significantly reduced final paw weights compared to KINERET® QD treatment (p<0.006). (FIG. 11)

Change in body weight was significantly increased toward normal for rats treated with 100 mg/kg KINERET® QD (54%), 120 mg/kg Met-I10-TripT-IL1ra QD (49%), or 120 mg/kg GG-V17-TripT-IL1ra QD (65%), as compared to vehicle treated disease control animals.

### Example 6

### Streptozocin (STZ)-Induced Diabetes Model

STZ (Sigma Aldrich) was administered once daily for five successive days at 50 mg/kg i.p. to fasted C57BL/6J male mice. The mice gradually developed higher levels of blood glucose from Day 1 to Day 4. The levels rose from 6.9 nmol/L to 13.1 nmol/L during the STZ induction period. Five days (Day 4) after the last STZ dosing, the mice were randomly distributed into 10 treatment groups each containing 10 mice in good condition. Treatment started on this day, before onset of diabetes and continued beyond the onset. The treatment groups were as shown in Table 5.

TABLE 5

| Group | Induction of | | Dose | |
|---|---|---|---|---|
| No. | Diabetes | Test Article | mg/kg | Administration |
| 1 | + | Vehicle | 0 | i.p. once daily |
| | | | | (QD) |
| 2 | + | KINERET ® | 100 | i.p. once daily |
| | | | | (QD) |
| 3 | + | KINERET ® | 30 | i.p. once daily |
| | | | | (QD) |
| 4 | + | I10-TripT-IL1-RA | 100 | i.p. once daily |
| | | | | (QD) |
| 5 | + | I10-TripT-IL1-RA | 30 | i.p. once daily |
| | | | | (QD) |
| 6 | + | I10-TripT-IL1-RA | 100 | i.p. twice |
| | | | | weekly (QD) |
| 1 | + | Vehicle | 0 | i.p. once daily |
| | | | | (QD) |
| 2 | + | KINERET ® | 100 | i.p. once daily |
| | | | | (QD) |
| 3 | + | KINERET ® | 30 | i.p. once daily |
| | | | | (QD) |
| 4 | + | I10-TripT-IL1-RA | 100 | i.p. once daily |
| | | | | (QD) |
| 5 | + | I10-TripT-IL1-RA | 30 | i.p. once daily |
| | | | | (QD) |
| 6 | + | I10-TripT-IL1-RA | 100 | i.p. twice |
| | | | | weekly (QD) |

The study period was 28 days and the mice were weighed once weekly during the treatment period. Blood glucose levels were measured every other day during the study period in order to monitor development of diabetes. A droplet of whole blood was collected by tail vein bleeding and placed on an Ascensia ELITE® blood glucose test strip and analyzed with an Ascensia ELITE® blood glucose meter (Bayer). The values were recorded, and x-fold increase in any given group compared to levels at treatment initiation was calculated. Clinical symptoms were observed daily or as appropriate in groups where adverse symptoms occurred.

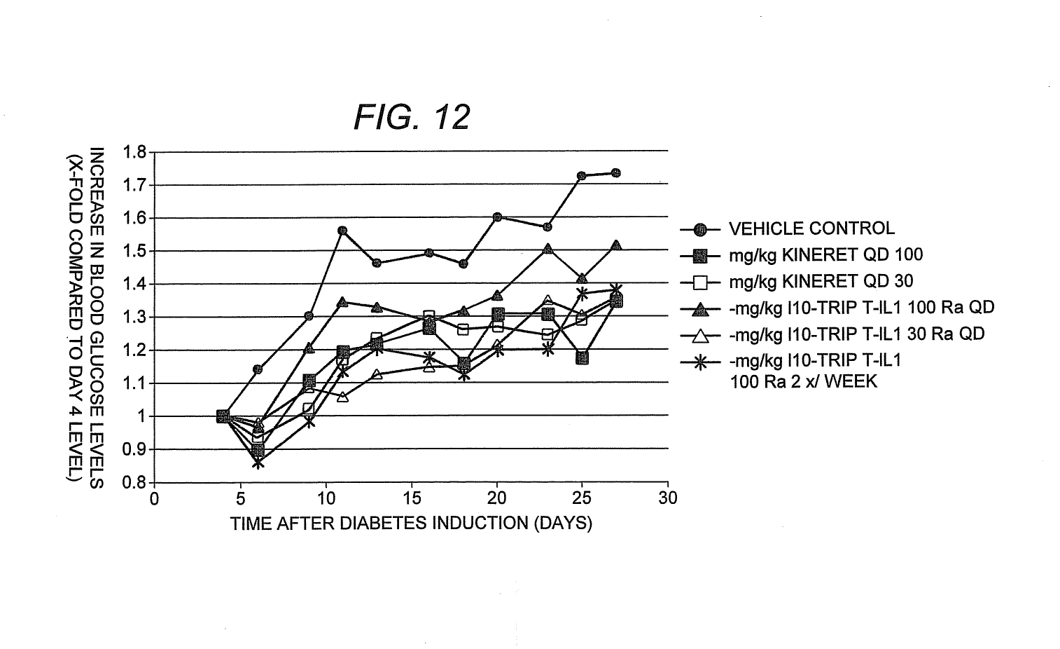

As shown in FIG. 12, a marked reduction of blood glucose levels was observed after daily i.p. dosing of either I10-TripT-IL1-Ra or KINERET® at both 100 and 30 mg/kg. Furthermore, twice weekly dosing of 100 mg/kg 110-TripT-IL1Ra was equally effective as daily dosing of 100 mg/kg KINERET®. These data demonstrate that trimerized IL-1Ra is an effective treatment of experimentally induced diabetes.

### Example 8

### Calorimetric Stability Analysis of Full-Length and Truncated Tetranectin-Derived Trimerisation Unit

Differential scanning calorimetry (DSC) is a technique to study thermally induced transitions and particularly, the conformational transition of biologic macromolecules including, for example, measurement between folded and unfolded structures of a protein. A characterization of the thermal stability of the trimerisation domain of tetranectin was obtained using DSC, both of the full length trimerisation domain and of deletion mutants.

The DSC scans were conducted on a VP-DSC from MicroCal LLC (Northampton, Mass.). For most of the scans a PBS buffer containing 110 mM NaCl and 40 mM NaPO₄ was used. The pH was 7.4. The scans were made from 10 to 110° C. under approximately 25 psi at a scan rate of 1° C./min and a pre-scan period of 20 minutes. At least two scans with buffer in both chambers were conducted before removal of buffer from both chambers and the filling with new buffer and protein/peptide solution to the reference and sample chamber respectively.

Peptides were synthesized by an outside vendor and were based on the tetranectin sequence, however, minor modifications were applied. In particular, the residue corresponding to Cys 50 was changed to a Serine. Also, residue number 28 is in native TN polymorphic, being either a Ser or an Ala. In the following experiments, Ala was selected. In addition, the N and C-terminal of the peptides were modified to remove charge and mimic natural peptides, increase stability toward digestion by aminopeptidases and block against synthetase activity. In the N-terminal deletion class peptides the N-terminal is acetylated and the C-terminal is prolonged with a β-Ala and a Cys. For the C-terminal deletion class peptides the modification are amidation of the C-terminal and all the sequences start with Cys-βAla.

Since these peptides were lyophilized from a TFA solution the pH of the redissolved peptides had to be adjusted. This was done using our micro-pH meter from Unisense. The pH was adjusted to the same mV value as found for the buffer by adding small portions of 0.2 M NaOH to the solution immediately followed by mixing. In order to prevent disulphide-bridge dimerisation and possible stabilization of the multimeric structures all scanning of peptides were conducted in 5 mM β-mercaptoethanol. Prior to scanning and addition of β-mercaptoethanol the sample and buffer were extensively degassed under vacuum while stirring.

### Concentration Dependence of the Stability

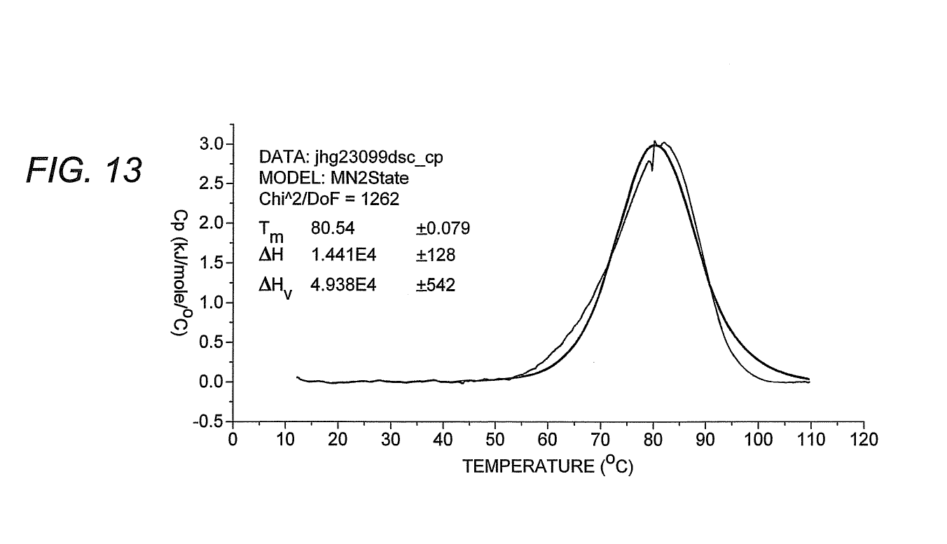

Trip-A (SEQ ID NO:42) was scanned at concentrations ranging from 2 mM to 0.125 mM, by lowering the concentration by a factor 2. This corresponds to protein concentrations from 12.7 mg-ml to 0.80 mg/mL. The data fitted to the non-2-state model and the “dissociation with dCₚ” model, but did not fit well to the two-state model. The difference between the two-state model and the non-2-state model can be stated as that the non-2-state model contains a contribution to the unfolding energy from co-operative stabilization of subunits. This leads to a sharpening of the peak. The parameter introduced relative to the 2-state model is called the ΔHᵥ, or the van't Hoff enthalpy. The ratio between ΔHᵥ and ΔH can be taken as a measurement of the number of subunits in one unfolding unit. The result is shown on FIG. 13 and Table 6. The data is from one representative scan and fitted to the non-2-state model.

TABLE 6
Concentration dependence of Trip-A on the stability and
unfolding parameters using the non-2-state model

| | | | ΔHᵥ | | Reversibility | |
|---|---|---|---|---|---|---|
| Conc (mM) | T_{M} (° C.) | ΔH (kJ/mol) | (kJ/mol) | ΔHᵥ/ΔH | (%) | χ²/Dof |
| 2.0 | 90.4 ± 0.1 | 32.4 ± 0.3 | 279.9 ± 3.5 | 8.6 | 105 (!) | 612 |
| 1.0 | 86.5 ± 0.1 | 36.9 ± 0.6 | 272.2 ± 5.4 | 7.4 | 97.7 | 1971 |
| 0.5 | 81.4 ± 0.1 | 48.6 ± 0.4 | 232.6 ± 2.4 | 4.8 | 94.1 | 805 |
| 0.25 | 75.9 ± 0.1 | 53.3 ± 0.4 | 192.6 ± 1.7 | 3.6 | 95.6 | 588 |
| 0.125 | 73.1 ± 0.03 | 63.7 ± 0.2 | 177.9 ± 0.6 | 2.8 | 94.2 | 2487 |

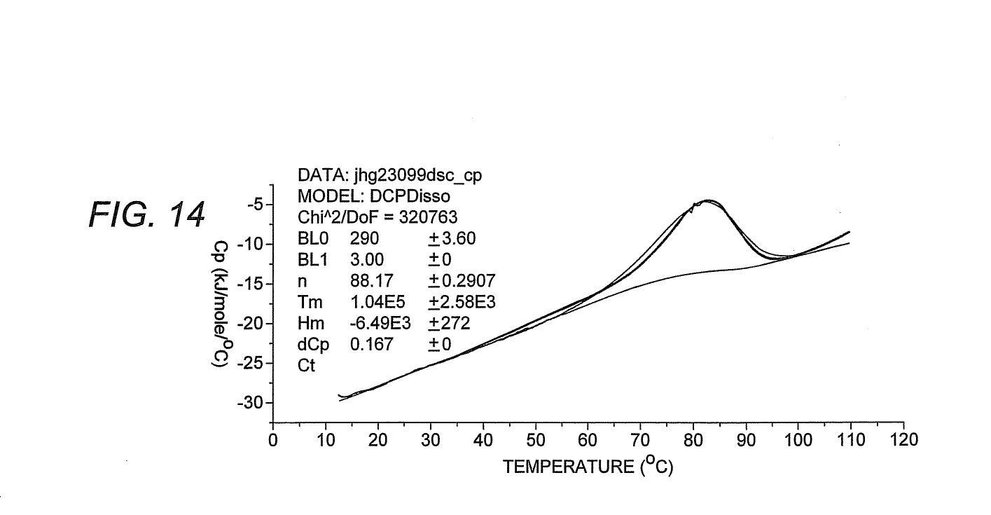

The “dissociation with dCₚ” model describes a system where unfolding and dissociation of a multisubunit protein is simultaneous. The data and a fit found for fitting to that model is given in Table 7 and FIG. 14. The data is from one representative scan and fitted to the “dissociation with dCp” model. The ΔHm and dCp value is per trimer.

TABLE 7
Concentration dependence of Trip-A on the stability and
unfolding parameters using the “dissociation with dCp” model

| Conc | T_{M} (° C.) | | dCₚ | Rev. | | | T_{M} if N |
|---|---|---|---|---|---|---|---|
| (mM) | (N = 3) | ΔHₘ (kJ/mol) | (kJ/molK) | (%) | χ²/Dof | N (free) | is free |
| 2.0 | 85.2 ± 0.4 | 384.5 ± 3.4 | −7.1 ± 0.5 | 95.6 | 124446 | 5.67 | 77.0 ± 0.5 |
| 1.0 | 83.2 ± 0.1 | 303.3 ± 0.6 | 0.58 ± 0.02 | 97.6 | 3436 | 2.21 | 86.0 ± 0.1 |
| 0.5 | 84.7 ± 0.1 | 319.2 ± 0.4 | 0.67 ± 0.01 | 97.6 | 1446 | 3.93 | 83.5 ± 0.1 |
| 0.25 | 81.4 ± 0.1 | 284.5 ± 0.7 | 3.3 ± 0.1 | 98.2 | 3965 | 2.48 | 81.2 ± 0.1 |
| 0.125 | 82.2 ± 0.3 | 277.0 ± 1.9 | 2.4 ± 0.2 | 98.9 | 7651 | 1.88 | 78.9 ± 0.3 |

Both the non-2-state model and the dissociation of dCp model provide acceptable fits, and both can describe the dissociation of a multimeric system. The dissociation with dCp model is somewhat more complex mathematically, with more parameters to vary, and even two free flowing constants with no physical relation (BL0 and BL1). Therefore the non-2-state model is the simplest model. The non-2-state model, however, provides a concentration dependent variation of the parameters, which are not seen for the dissociation with dCp-model. It is a general problem to discriminate between these two models, and furthermore it is not generally agreed how to interpret the dCₚ values obtained in relation to the proteins structure (See Haynie, D T, in Biocalorimtry, applications of calorimetry in the biological sciences, Ladbury J E and Chowdhry B Z, eds., Wiley, Chichester, 1998, pp 183-205). Accordingly, all the following data is based on the “dissociation with dCp-model.”

Scanning of Trip-A at different concentrations

Trip-A exhibited Tₘ values ranging from 81-85° C. with no clear correspondence between that value and the concentration of the construct as seen in Table 7. ΔHₘ, is also rather constant at approximately 300 kJ/mole. For the 2 mM scan, however, the value is somewhat higher. The only obvious reason for that being the problems for making a nice baseline, since the peak is quite big, and almost extends to the endpoint of the scan. It should be noted that dCₚ varies with no obvious pattern in relation to the concentration. Accordingly, there seems to be no trimer-trimer interactions taking place at higher concentrations stabilizing the overall structure of the protein.

Up and Down Scanning of Trip-A

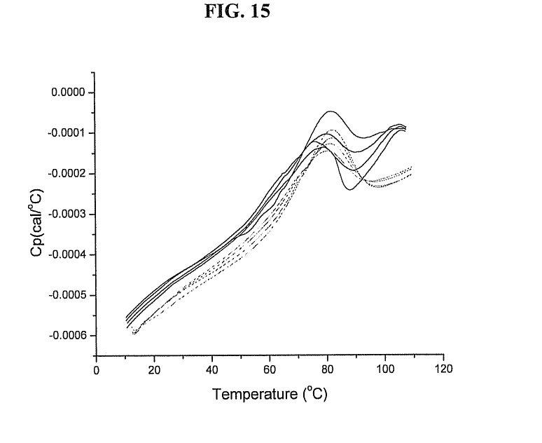

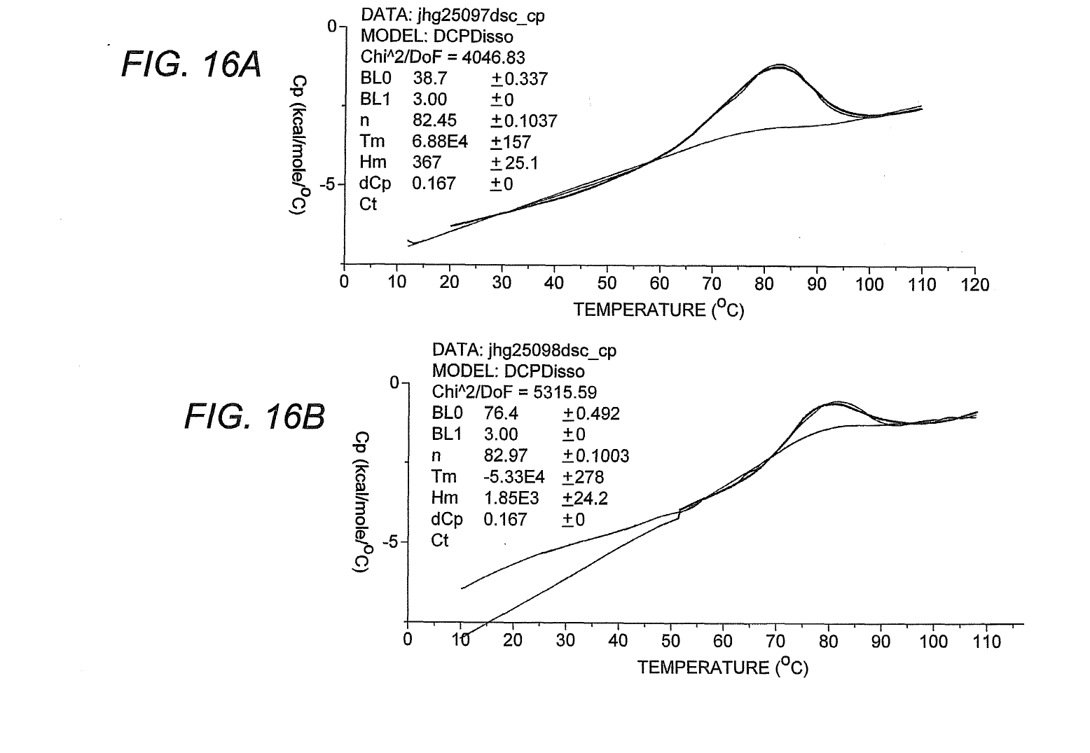

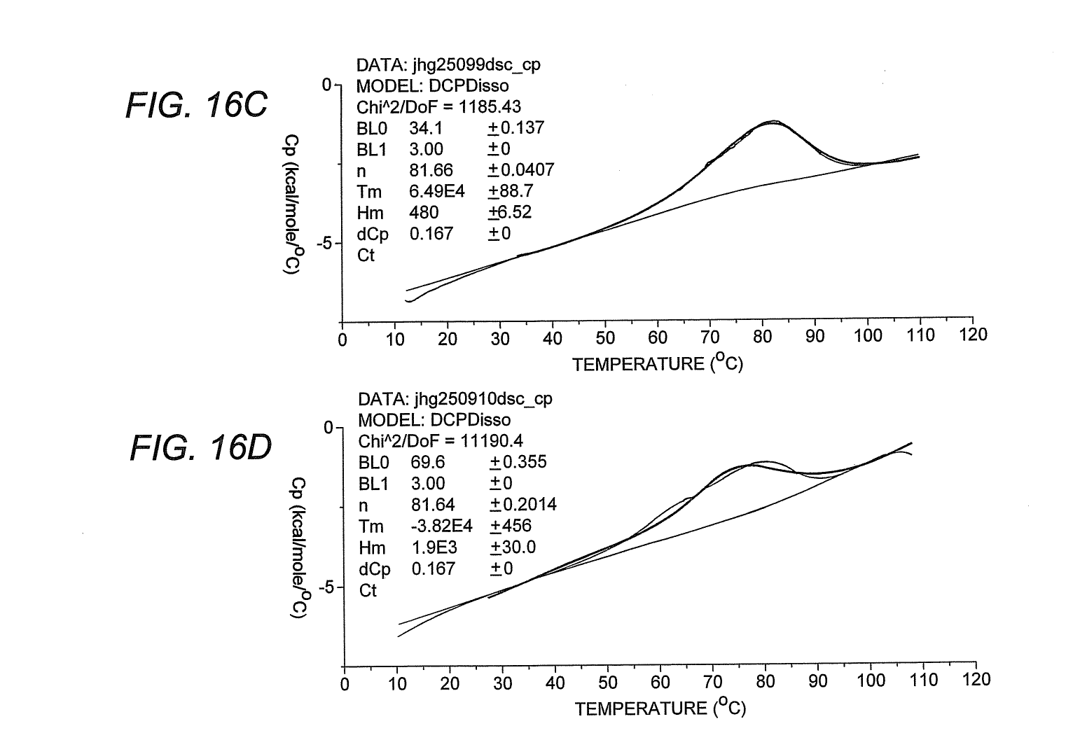

Trip-A was scanned at a concentration of 0.5 mM. Table 8 shows the fitted thermodynamic parameters, while FIGS. 15 and 16A-D show the scans and fits. In general this underlines the reversibility of the Trip-A folding. However, the refolding experiments were somewhat difficult to fit, again due to baseline problems. Nevertheless, the data were easier to fit and gave more consistent values (e.g., constant Tₘ values between scans). In theory the parameters should be identical if the process was 100% reversible, and they come considerably closer to that using the dissociation with dCₚ model. Table 8 shows the fitted thermodynamic parameters, while FIGS. 15 and 16A-D show the scans and fits. 16A and C are up scans, and B and D are down scans.

TABLE 8
Thermodynamic parameters for the folding and unfolding of Trip-A.

| | | ΔHₘ | dCₚ | Rev. | | N | T_{M} if N |
|---|---|---|---|---|---|---|---|
| | T_{M} (° C.) | (kJ/mol) | (kJ/molK) | (%) | χ²/Dof | (free) | is free |
| Up, first | 82.3 ± 0.1 | 288.3 ± 0.6 | 1.7 ± 0.1 | 94.2 | 3218 | 2.13 | 84.0 ± 0.1 |
| Up, second | 81.7 ± 0.1 | 271.5 ± 0.3 | 2.0 ± 0.1 | 94.0 | 1185 | 3.7 | 80.3 ± 0.1 |
| Down, first | 79.8 ± 0.1 | −249.0 ± 1.3 | 3.5 ± 0.1 | 68.9 | 7916 | 0.66 | 81.4 ± 0.4 |
| Down, sec. | 80.2 ± 0.2 | −171.5 ± 2.2 | 6.9 ± 0.1 | 85.6 | 12154 | 1.45 | 85.6 ± 0.3 |

The ΔHm and dCp values are per trimer.

Stability of the Trip-A structure at low pH

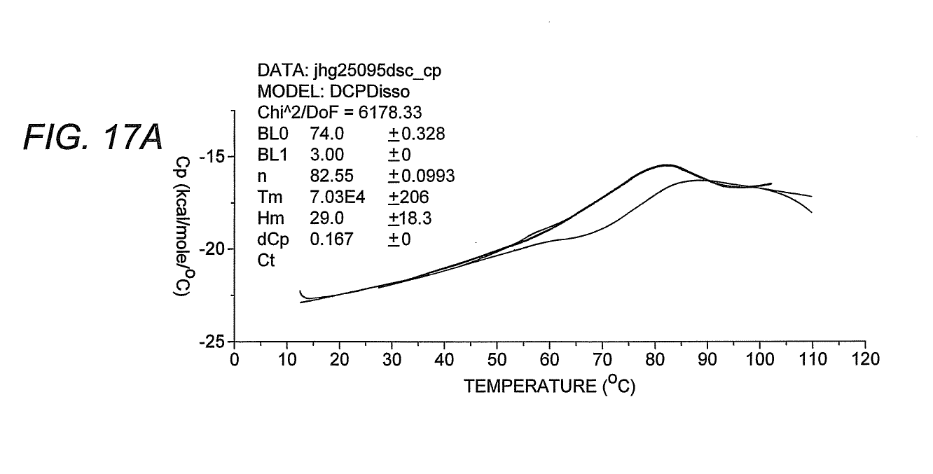

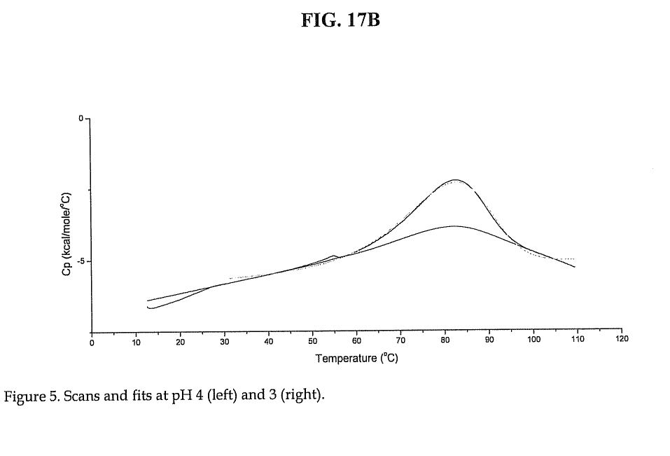

Trip-A's thermal stability was also tested at pH 3.0 and 4.0. The buffers used were 100 mM NaCl 25 mM NaPO₄, pH 3.0 or 100 mM NaCl 25 mM NaAcetat pH 4.0. Lyophilized protein was redisolved in the buffer and the pH checked using the micro-pH-meter. Table 4 shows the thermodynamic parameters found and FIGS. 17A and B each show tlree representative scans at pH 3, 4 and 7.4 with fits.

TABLE 9
Thermodynamic parameters for Trip-A when scanning at low pH values.

| | | | dCₚ | Rev | |
|---|---|---|---|---|---|
| pH | T_{M} (° C.) | ΔHₘ (kJ/mol) | (kJ/molK) | (%) | χ²/Dof |
| 3.0 | 84.6 ± 0.2 | 297.9 ± 2.9 | 11.9 ± 0.3 | 93.1 | 55950 |
| 4.0 | 82.6 ± 0.1 | 294.1 ± 0.9 | 0.12 ± 0.07 | 88.1 | 6178 |
| 7.4 | 84.7 ± 0.1 | 319.2 ± 0.4 | 0.67 ± 0.05 | 97.6 | 1446 |

The ΔHm and dCp values are per trimer.

The data show that Trip-A exhibits stability to pH, and the variation on Tₘ by changing the pH is not substantially bigger than the variation between different scans. Also note that ΔHₘ does not change much as effect of the pH.

Scanning the Deletion Constructs of the Trimerisation Domain

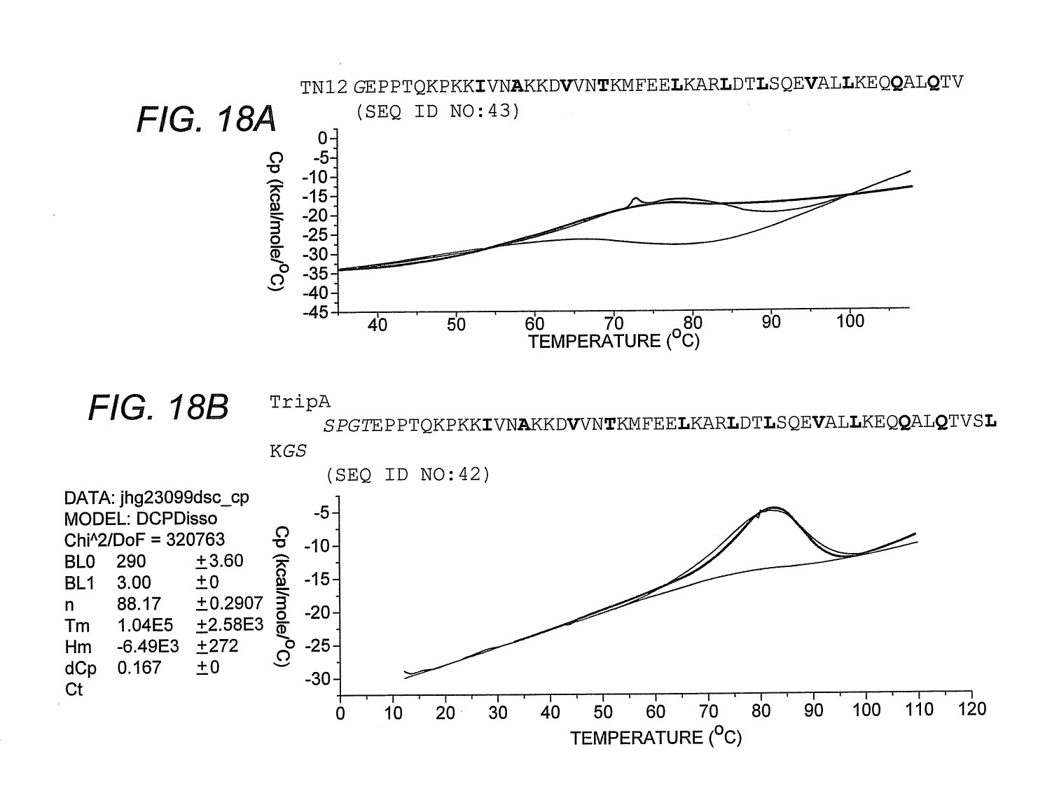

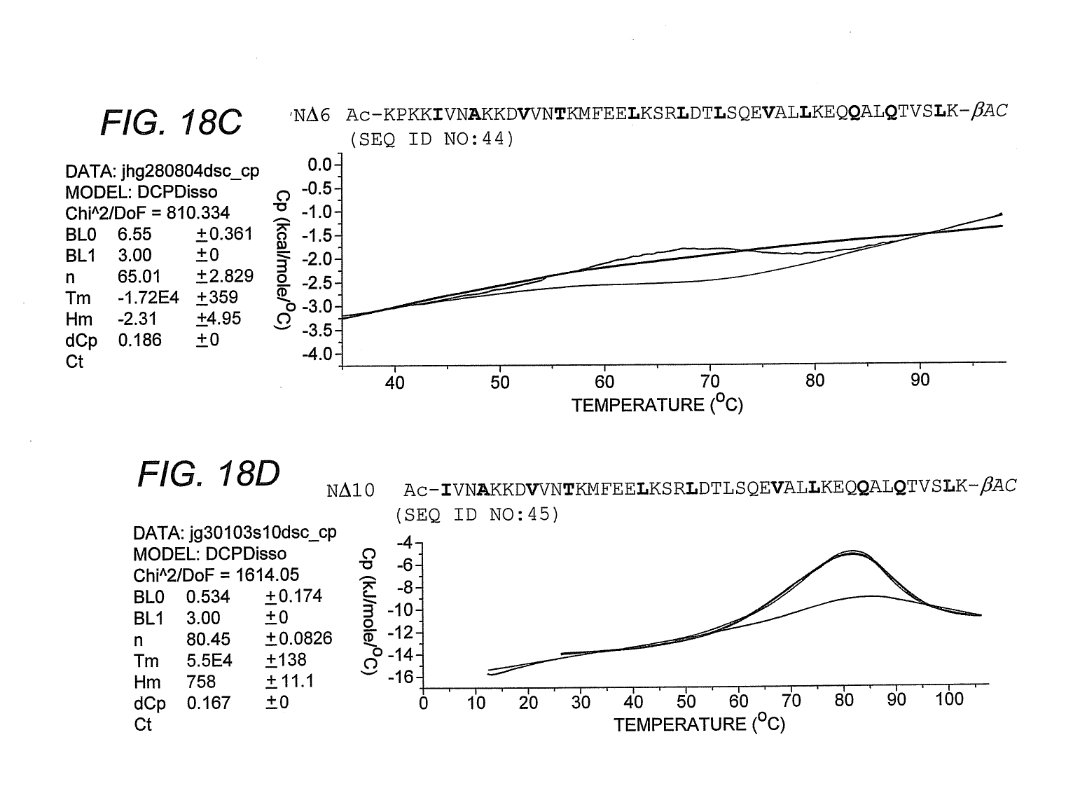

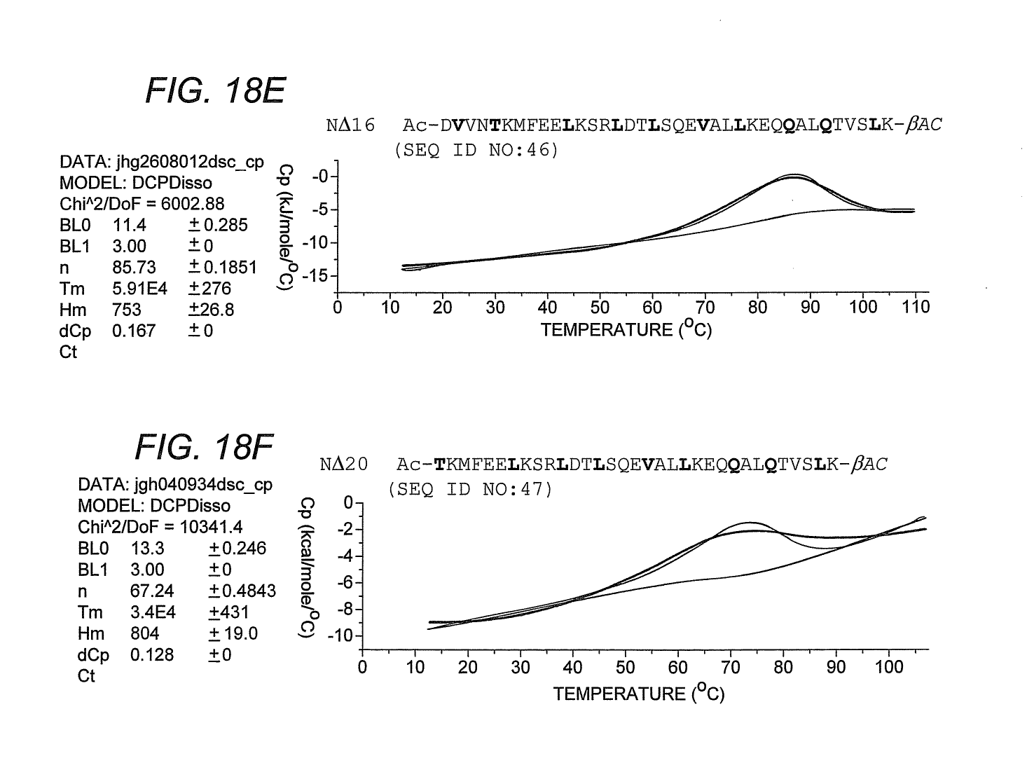

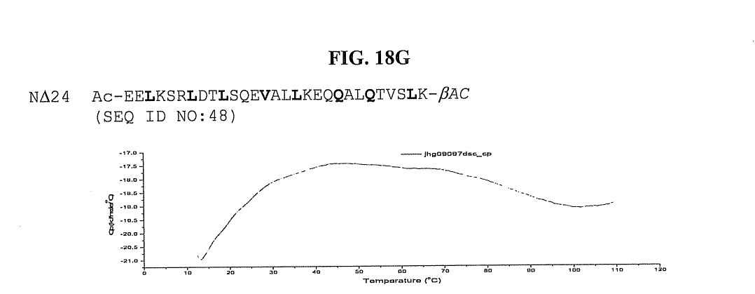

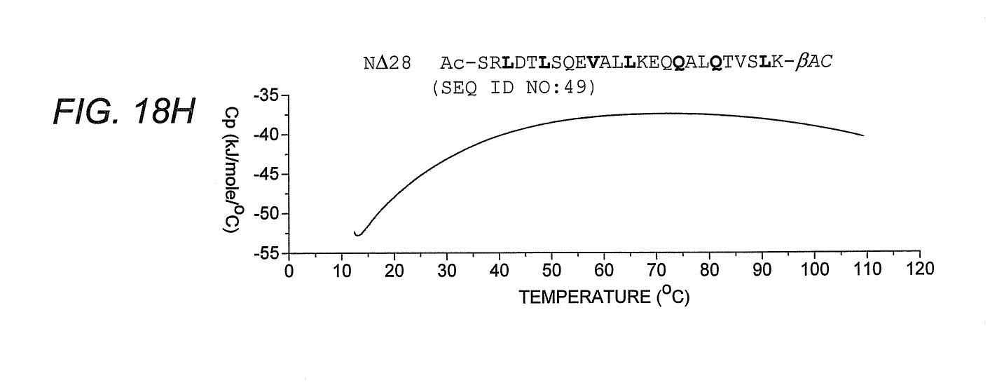

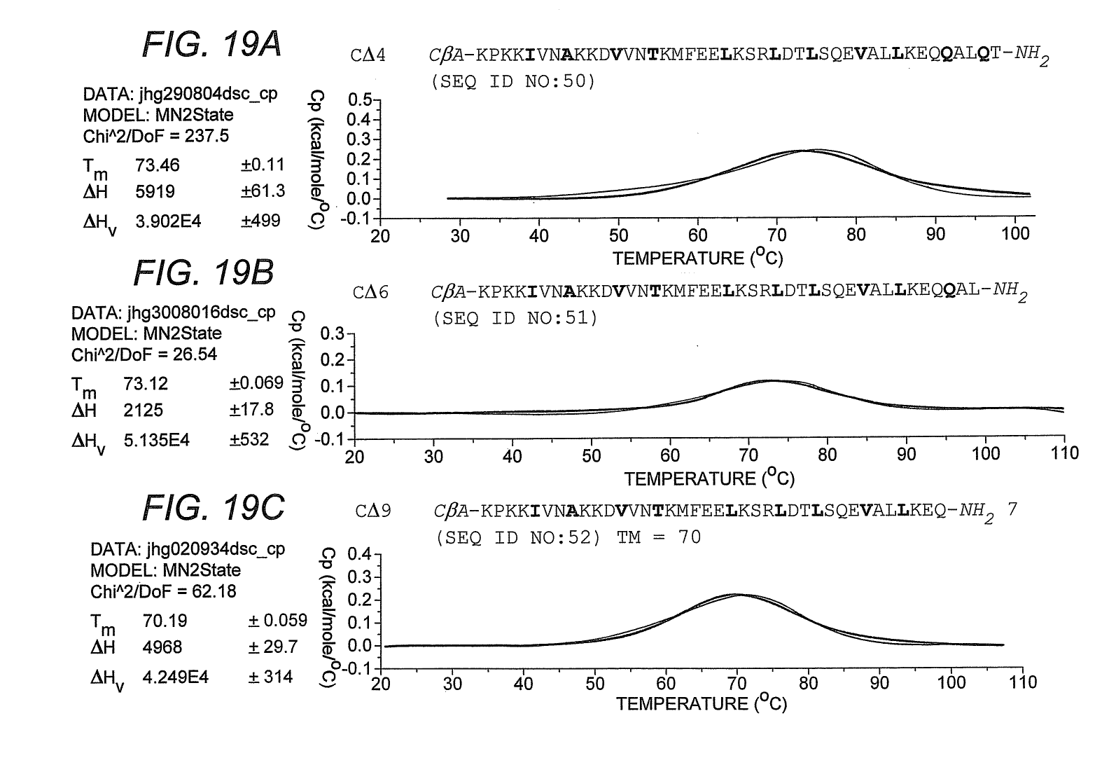

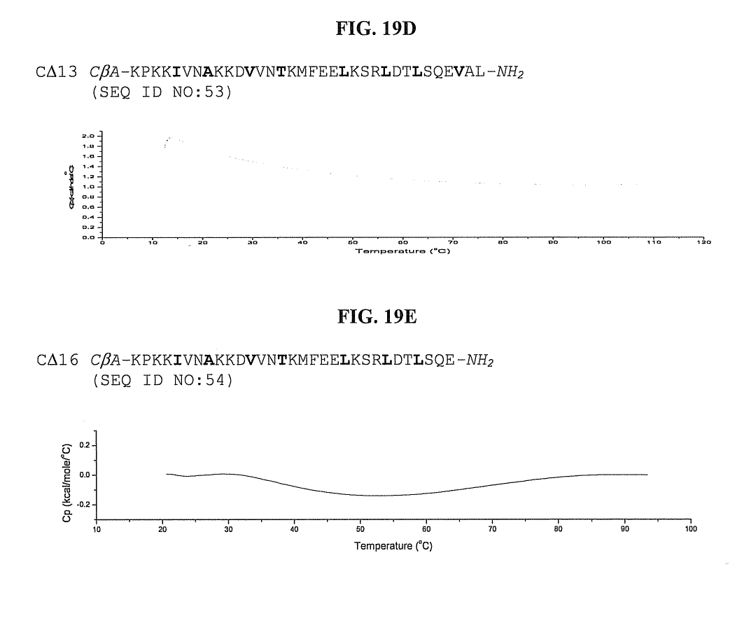

In general all the peptides were easily dissolved in the PBS buffer used. Table 9 and 5 summarizes the thermodynamic parameters found for N and C-terminal deletions, respectively. Representative scans are shown in FIGS. 18 and 19, for N and C terminal deletion constructs, respectively. The scans chosen were not all at 0.5 mM, which should understood when comparing small variations between constructs. All scans are made in the same buffer. β-mercaptoethanol added to a concentration of 5 mM for all constructs except Trip-A and TN12. All scans were here fitted to the dissociation with dCₚ model. For those structures that revealed unfolding peaks reversibility greater than 85% was observed, with the C-terminal deletion constructs exhibiting the lowest degree of reversibility.

The N-Terminal Deletion Constructs

Deletions trough the N-terminal of the trimerisation domain revel several interesting points. First, starting at Lys 6 is not optimal for stability, since it affects the stability negatively and also leads to much smaller unfolding energies and lower degree of reversibility, and thus hampers the data analysis. Furthermore better fits could be obtained by letting the N-value flow, and it would then drop to 0.11. This could indicate that the a model does not describe the unfolding of this peptide. However, it can be seen from the raw data that some kind of transition is taking place, thus indicating structure. The C-terminal constructs all start at Lys 6.

Second the transition from being able to observe an unfolding and to not unfolding is rather sharp. In general, the stable constructs have Tₘ values of about 80° C., the peptide NΔ20, starting at Thr 20, exhibits a Tₘ of 67° C., but deleting just 4 more residues leads to no observable unfolding. In conclusion deleting 10 to 16 residues does not affect the stability of the constructs, but removing only a part of the N-terminal lowers stability. This could indicate that the first 10 amino acid residues of TN forms some independent structure and that destroying that structure without removing it affects the overall stability of the construct.

TABLE 10
Thermodynamic parameters for the N-terminal deletion peptides

| | | T_{M} | ΔHₘ | dCₚ | | |
|---|---|---|---|---|---|---|
| Construct | Conc | (° C.) | (kJ/mol(trimer)) | (kJ/molK) | Rev. (%) | χ²/Dof |
| Trip-A | 0.5 | 84.7 ± 0.1 | 319.2 ± 0.4 | 0.7 ± 0.1 | 97.6 | 1446 |
| TN12 | 0.63 | 69.3 ± 0.5 | 191.2 ± 3.9 | 5.4 ± 0.2 | 117.0 | 56080 |
| NΔ6 (A1)^{#} | 0.56 | 63.8 ± 4.1 | 89.1 ± 5.1 | −1.3 ± 0.1 | 98.1 | 311985 |
| NΔ10 (AA5) | 0.5 | 80.5 ± 0.1 | 230.1 ± 0.6 | 3.2 ± 0.1 | 103.6 | 1612 |
| NΔ16 (A2) | 0.5 | 85.6 ± 0.1 | 247.2 ± 1.1 | 3.3 ± 0.1 | 101.1 | 6029 |
| NΔ20 (A3) | 0.38 | 67.2 ± 0.5 | 142.3 ± 1.8 | 3.4 ± 0.1 | 115.9 | 10341 |

| | | |
|---|---|---|
| NΔ24 (A4) | 0.32 | No structure |
| NΔ28 (B1) | 0.5 | No structure |

The ΔHm and dCp values are per trimer.
^{#}The data for this construct did not fit very well to the dissociation with dCp model if N was locked at 3, but by letting N flow a nice fit was obtained with a Tm of app. 82° C., but with N equal 0.1.

The C-Terminal Deletion Constructs

The stability found for all the C-terminal deletion constructs is somewhat lower than found for the N-terminal deletions. This is probably an effect of all the constructs starting at Lys 6. However, as seen for the N-terminal deletion constructs, the transition from structure to no observable structure is sharp, and actually sharper for the C-terminal deletion constructs. The stability of the constructs is not significantly altered before total breakdown of observable structure. That breakdown is induced when going from the CΔ9 to the CΔ13 construct by removing the residues Leu40-Lys41-Glu42-Gln-43. Leu 40 is the first hydrophobic residue at an a or d position when going up from the C-terminal, when excluding Leu 51.

TABLE 11
Thermodynamic parameters for the C-terminal deletion peptides.

| | | T_{M} | ΔHₘ | dCₚ | | |
|---|---|---|---|---|---|---|
| Construct | Conc | (° C.) | (kJ/mol(trimer)) | (kJ/molK) | Rev. (%) | χ²/Dof |
| Trip-A | 0.50 | 84.7 ± 0.1 | 319.2 ± 0.4 | 0.7 ± 0.1 | 97.6 | 1446 |
| TN12 | 0.63 | 69.3 ± 0.5 | 191.2 ± 3.9 | 5.4 ± 0.2 | 117 | 56080 |
| NΔ6 (A1)^{#} | 0.56 | 63.8 ± 4.1 | 89.1 ± 5.1 | −1.3 ± 0.1 | 98.1 | 311985 |
| CΔ4 (D3) | 0.6 | 63.6 ± 0.2 | 106.7 ± 0.9 | 4.1 ± 0.1 | 96.4 | 961 |
| CΔ6 (D2) | 0.6 | 66.4 ± 0.4 | 125.1 ± 1.1 | 1.7 ± 0.1 | 96.3 | 1724 |
| CΔ9 (D1) | 0.6 | 62.1 ± 0.3 | 131.3 ± 1.3 | 2.9 ± 0.1 | 103.8 | 3224 |

| | | |
|---|---|---|
| CΔ13 (C4) | 0.5 | No structure |
| CΔ15 (C3) | 0.4 | No structure |

The ΔHm and dCp value is per trimer.
^{#}The data for this construct did not fit very well to the dissociation with dCp model if N was locked at 3, but by letting N flow a fit was obtained with a Tm of approximately 82° C., but with N equal 0.1.

### Example 9

The following example summarizes the thermal stability of the Trimeric IL-1Ra Met-1-10-TripT and Met-V-17-TripT differential scanning calorimetry (DSC). The trimerizing domain of these polypeptides include I10 and V17. The N-terminal methionine residue is a result of the production system.

The DSC scans were conducted on a VP Capillary DSC from MicroCal. All of the scans were performed in PBS buffer containing 110 mM NaCl and 40 mM NaPO₄, pH 7.4. The scans were made from 30 to 110° C. under approximately 0.55 psi at a scan rate of 5° C./min and a pre-scan equilibration of 20 minutes. Only up-scanning was conducted. The capillary DSC was equilibrated with PBS buffer and all samples were bracketed with PBS buffer for baseline subtraction. Both Met I-10-TripT (SEQ ID NO: 103) and Met V-17-TripT (SEQ ID NO: 104) were scanned at a concentration of 23.06 μm and 23.90 μm, respectively. This corresponds to a protein concentration of 500 μg/ml for each sample. The data was fitted to the non-2-state model.

Typical monomeric proteins follow a simple two state transition between folded and unfolded structures. The non-2-state model contains a contribution to the unfolding energy from co-operative stabilization of subunits and results in a multistate process which most likely has an unfolding intermediate (i.e. non-2 state process). The parameters introduced relative to the 2-state model is called the ΔHv, or the van't Hoff enthalpy. The ratio between ΔHᵥ and ΔH can be taken as a measurement of the number of subunits in one unfolding unit.

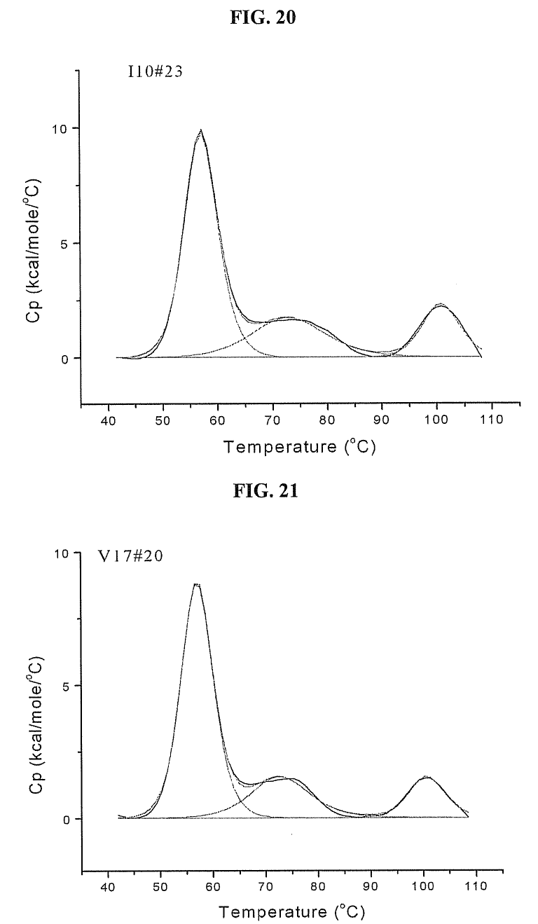

It should be noted that this technique is limited by assessment of protein concentration dependency, which was not evaluated in this study. The results are shown in Table 1 and the thermograms with the non 2-state model fit are shown in FIGS. 20 and 21. For both samples three T_{M} values were observed. The values are concentration dependent and therefore the T_{M} values are relative, not absolute, due to the contributions from the stabilization from the mutimers. 2

TABLE 12
Thermodynamic parameters for unfolding of
trimeric IL-1Ra Met I-10 and Met V-17

| Sample/ | | | ΔH_{V} | |
|---|---|---|---|---|
| concentration | T_{M} (° C.) | ΔH (kJ/mol) | (kJ/mol) | ΔHv/ΔH |
| I-10 | 57.13 ± 0.03 | 78.8 ± 0.87 | 107.5 ± 1.18 | 1.36 |
| 23.06 μm | 73.04 ± 0.26 | 31.0 ± 1.16 | 53.62 ± 2.58 | 1.73 |
| | 100.8 ± 0.12 | 20.7 ± 0.64 | 124.5 ± 4.85 | 6.01 |
| V-17 | 57.08 ± 0.02 | 71.1 ± 0.53 | 108.2 ± 0.89 | 1.52 |
| 23.90 μm | 72.81 ± 0.18 | 23.0 ± 0.68 | 63.83 ± 2.42 | 2.77 |
| | 100.7 ± 0.13 | 14.0 ± 0.45 | 122.7 ± 4.97 | 8.76 |

While it is generally considered that the trimerizing tetranectin polypeptide will form stable trimers above 60° C., the inventors have identified a concentration dependency when using the non 2-state model (see Example 8, Table 6, above). This experiment was conducted with a polypeptide concentration that were about 6-fold lower than the experiments reported in Table 6, and further reflect the concentration dependence of the ability of the polypeptides to trimerize. Here, it is shown that even at very low concentrations, the polypeptides will still trimerize at close to 60° C.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in molecular biology, immunology, chemistry, biochemistry or in the relevant fields are intended to be within the scope of the appended claims.

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is to be noted that, as used herein and in the appended claims, the singular forms “a,” “an,” and “the” include the plural reference unless the context clearly dictates otherwise. Thus, for example, a reference to “a linker” is a reference to one or more linkers and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Any numerical values recited herein include all values from the lower value to the upper value in increments of one unit provided that there is a separation of at least two units between any lower value and any higher value. As an example, if it is stated that the concentration of a component or value of a process variable such as, for example, size, angle size, pressure, time and the like, is, for example, from 1 to 90, specifically from 20 to 80, more specifically from 30 to 70, it is intended that values such as 15 to 85, 22 to 68, 43 to 51, to 32, etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Particular methods, devices, and materials are described, although any methods and materials similar or equivalent to those described herein can be used in the practice or testing of the invention. The disclosures of all references and publications cited above are expressly incorporated by reference in their entireties to the same extent as if each were incorporated by reference individually.

What is claimed is:

1. An isolated polypeptide comprising an amino acid sequence of VNTKMFEELKSRLDTLAQEVALLKEQQALQTVCLK (SEQ ID NO:80) having:
(d) an amino-terminal truncation of 0 to 6 amino acid residues, and wherein three polypeptides form a trimeric complex;
(e) a carboxyl-terminal truncation of 0 to 15 amino acid residues, and wherein three polypeptides form a trimeric complex; or
(f) an amino-terminal truncation of 0 to 6 amino acid residues and a carboxyl-terminal truncation of 0 to 15 residues, and wherein three polypeptides form a trimeric complex.

2. The isolated polypeptide of claim 1 wherein the N terminus is T20

3. The isolated polypeptide of claim 1 wherein the C-terminus is selected from the group consisting of K52, V49, T48, Q47, L46, and Q43.

4. The isolated polypeptide of claim 1 having a cysteine to serine substitution at position 50.

5. An isolated polypeptide comprising an amino acid sequence of EPPTQIPKKIVNAKKDVVNTKMFEELKSRLDTLAQEVALLKEQQALQT (SEQ ID NO:62 having:
(a) an amino-terminal truncation of 0 to 23 amino acid residues, and wherein three polypeptides form a trimeric complex;
(b) a carboxyl-terminal truncation of 0 to 12 amino acid residues, and wherein three polypeptides form a trimeric complex; or
(c) an amino-terminal truncation of 0 to 23 amino acid residues and a carboxyl-terminal truncation of 0 to 12 residues, and wherein three polypeptides form a trimeric complex.

6. The isolated polypeptide of claim 5 wherein the N terminus is selected from the group consisting of K6, I10, D16, V17, and T20.

7. The isolated polypeptide of claim 5 wherein the C-terminus is one of T48, Q47, L46, and Q43.

8. An isolated polypeptide comprising an amino acid sequence of PPTQKPIKIVNAKIQDVVNTKMFEELKSRLDTLAQEVALLKEQQALQTV (SEQ ID NO:64) having:
(a) an amino-terminal truncation of 0 to 22 amino acid residues, and wherein three polypeptides form a trimeric complex;
(b) a carboxyl-terminal truncation of 0 to 13 amino acid residues, and wherein three polypeptides form a trimeric complex; or
(c) an amino-terminal truncation of 0 to 22 amino acid residues and a carboxyl-terminal truncation of 0 to 13 residues, and wherein three polypeptides form a trimeric complex.

9. The isolated polypeptide of claim 8 wherein the N terminus is selected from the group consisting of K6, I10, D16, V17, and T20.

10. The isolated polypeptide of claim 8 wherein the C-terminus is one of V49, T48, Q47, L46, and Q43.

11. An isolated polypeptide comprising an amino acid sequence that is at least 50% identical to the polypeptide of any one of claims 1, 5 and 8.

12. An isolated polypeptide comprising an amino acid sequence that is at least 60% identical to the polypeptide of any one of claims 1, 5 and 8.

13. An isolated polypeptide comprising an amino acid sequence that is at least 70% identical to the polypeptide of any one of claims 1, 5 and 8.

14. The polypeptide of any one of claims 1, 5 and 8 comprising a serine to alanine substitution at position 28.

15. The trimeric polypeptide complex comprising three polypeptides of any one of claims of claim 1, 5 and 8.

16. A fusion protein comprising any one of the polypeptides of claims 1, 5 and 8 and a therapeutic polypeptide.

17. A trimeric complex comprising three fusion proteins of claim 16.

18. The fusion protein of claim 16, further comprising polyethylene glycol.

19. The fusion protein of claim 16, further comprising a linker between the therapeutic polypeptide and the trimerizing domain.

20. An isolated polynucleotide encoding the polypeptide of any of claims of claim 1, 5 and 8.

21. A vector comprising the polynucleotide of claim 20.

22. A host cell comprising the vector of claim 21

23. The host cell of claim 22 wherein the cell is a mammalian cell.

24. A pharmaceutical composition comprising the fusion protein of claim 16 and at least one pharmaceutically acceptable excipient.

25. A pharmaceutical composition comprising the trimeric complex of claim 17 and least one pharmaceutically acceptable excipient.

26. The polypeptide of any one of claim 5 and 8 wherein the polypeptide includes residue T4, and wherein the polypeptide is expressed in a mammalian cell.
